SEQUENCE LISTING (30 sequences)

SEQ ID NO: 1 - protein, 666 aa, Homo sapiens
  REGION 1..666: note = Human SEMA7A
MTPPPPGRAAPSAPRARVPGPPARLGLPLRLRLLLLLWAAAASAQGHLRSGPRIFAVWKGHVGQDRVDFGQTEPHTVLFHEPGSSSVWVGGRGKVYLFDFPEGKNASVRTVNIGSTKGSCLDKRDCENYITLLERRSEGLLACGTNARHPSCWNLVNGTVVPLGEMRGYAPFSPDENSLVLFEGDEVYSTIRKQEYNGKIPRFRRIRGESELYTSDTVMQNPQFIKATIVHQDQAYDDKIYYFFREDNPDKNPEAPLNVSRVAQLCRGDQGGESSLSVSKWNTFLKAMLVCSDAATNKNFNRLQDVFLLPDPSGQWRDTRVYGVFSNPWNYSAVCVYSLGDIDKVFRTSSLKGYHSSLPNPRPGKCLPDQQPIPTETFQVADRHPEVAQRVEPMGPLKTPLFHSKYHYQKVAVHRMQASHGETFHVLYLTTDRGTIHKVVEPGEQEHSFAFNIMEIQPFRRAAAIQTMSLDAERRKLYVSSQWEVSQVPLDLCEVYGGGCHGCLMSRDPYCGWDQGRCISIYSSERSVLQSINPAEPHKECPNPKPDKAPLQKVSLAPNSRYYLSCPMESRHATYSWRHKENVEQSCEPGHQSPNCILFIENLTAQQYGHYFCEAQEGSYFREAQHWQLLPEDGIMAEHLLGHACALAASLWLGVLPTLTLGLLVH

SEQ ID NO: 2 - protein, 10 aa, synthetic construct
  REGION 1..10: note = Synthetic Construct; 10mer peptide of human SEMA7A
ADRHPEVAQR

SEQ ID NO: 3 - protein, 13 aa, synthetic construct
  REGION 1..13: note = Synthetic Construct; 10mer peptide of human SEMA7A with linker for conjugation
CGGADRHPEVAQR

SEQ ID NO: 4 - protein, 41 aa, synthetic construct
  REGION 1..41: note = Synthetic Construct; 41mer peptide of human SEMA7A
QPIPTETFQVADRHPEVAQRVEPMGPLKTPLFHSKYHYQKV

SEQ ID NO: 5 - protein, 124 aa, synthetic construct
  REGION 1..124: note = Synthetic Construct; Clone H1 Heavy Chain
QVKLQESGTVLARPGASVKMSCKASVYSFTSYWMHWVKQRPGQGLEWIGAIYPGNSDTSYNQKFKGKAKLTAVTSASTAYMELSSLTNEDSAVYYCTRGGAPTMITAYYAMDYWGQGTSVTVSS

SEQ ID NO: 6 - DNA, 372 bp, synthetic construct
  misc_feature 1..372: note = Synthetic Construct; Clone H1 Heavy Chain
  source 1..372: mol_type = other DNA
caagtcaagctgcaggagtctgggactgtgctggcaaggcctggggcttccgtgaagatgtcctgcaaggcttctgtctacagctttaccagctactggatgcactgggtaaaacagaggcctggacagggtctagaatggattggtgctatttatcctggaaatagtgatactagctacaaccagaagttcaagggcaaggccaaactgactgcagtcacatccgccagcactgcctacatggagctcagcagcctgacaaatgaggactctgcggtctattactgtacaagaggaggggcgcctactatgataacggcgtactatgctatggactactggggtcaaggaacctcagtcaccgtctcctca

SEQ ID NO: 7 - protein, 108 aa, synthetic construct
  REGION 1..108: note = Synthetic Construct; Clone H1 Light Chain
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIKR

SEQ ID NO: 8 - DNA, 324 bp, synthetic construct
  misc_feature 1..324: note = Synthetic Construct; Clone H1 Light Chain
  source 1..324: mol_type = other DNA
gatatccagatgacacagactacatcctccctgtctgcctctctgggagacagagtcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagattattctctcaccattagcaacctggagcaagaagatattgccacttacttttgccaacagggtaatacgcttccgtggacgttcggtggaggcaccaagctggaaatcaaacgg

SEQ ID NO: 9 - protein, 5 aa, synthetic construct
  REGION 1..5: note = Synthetic Construct; Clone H1 CDR-H1
SYWMH

SEQ ID NO: 10 - protein, 17 aa, synthetic construct
  REGION 1..17: note = Synthetic Construct; Clone H1 CDR-H2
AIYPGNSDTSYNQKFKG

SEQ ID NO: 11 - protein, 15 aa, synthetic construct
  REGION 1..15: note = Synthetic Construct; Clone H1 CDR-H3 (ta)
GGAPTMITAYYAMDY

SEQ ID NO: 12 - protein, 15 aa, synthetic construct
  REGION 1..15: note = Synthetic Construct; Clone H1 CDR-H3 (ss)
GGAPTMISSYYAMDY

SEQ ID NO: 13 - protein, 11 aa, synthetic construct
  REGION 1..11: note = Synthetic Construct; Clone H1 CDR-L1
RASQDISNYLN

SEQ ID NO: 14 - protein, 7 aa, synthetic construct
  REGION 1..7: note = Synthetic Construct; Clone H1 CDR-L2
YTSRLHS

SEQ ID NO: 15 - protein, 9 aa, synthetic construct
  REGION 1..9: note = Synthetic Construct; Clone H1 CDR-L3
QQGNTLPWT

SEQ ID NO: 16 - protein, 108 aa, synthetic construct; identical to SEQ ID NO: 7
  REGION 1..108: note = Synthetic Construct; Clone H7 Light Chain

SEQ ID NO: 17 - DNA, 324 bp, synthetic construct; identical to SEQ ID NO: 8
  misc_feature 1..324: note = Synthetic Construct; Clone H7 Light Chain
  source 1..324: mol_type = other DNA

SEQ ID NO: 18 - protein, 11 aa, synthetic construct
  REGION 1..11: note = Synthetic Construct; Clone H7 CDR-L1
RASQDISNYLN

SEQ ID NO: 19 - protein, 7 aa, synthetic construct
  REGION 1..7: note = Synthetic Construct; Clone H7 CDR-L2
YTSRLHS

SEQ ID NO: 20 - protein, 9 aa, synthetic construct
  REGION 1..9: note = Synthetic Construct; Clone H7 CDR-L3
QQGNTLPWT

SEQ ID NO: 21 - protein, 124 aa, synthetic construct
  REGION 1..124: note = Synthetic Construct; Clone H1 Heavy Chain (ss)
QVKLQESGTVLARPGASVKMSCKASVYSFTSYWMHWVKQRPGQGLEWIGAIYPGNSDTSYNQKFKGKAKLTAVTSASTAYMELSSLTNEDSAVYYCTRGGAPTMISSYYAMDYWGQGTSVTVSS

SEQ ID NO: 22 - protein, 124 aa, synthetic construct
EVQLQQSGTVLARPGASVKMSCKASGYSFTSYWMHWVKQRPGQGLEWIGAIYPGNSDTSYNQKFKGKAKLTAVTSASTAYMELSSLTNEDSAVYYCTRGGAPTMITAYYAMDYWGQGTSVTVSS

SEQ ID NO: 23 - protein, 107 aa, synthetic construct
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIK

SEQ ID NO: 24 - protein, 12 aa, synthetic construct
RGGAPTMITAYY

SEQ ID NO: 25 - DNA, 36 bp, synthetic construct
  source 1..36: mol_type = other DNA
agaggaggggcgcctactatgataacggcgtactat

SEQ ID NO: 26 - DNA, 36 bp, synthetic construct
  source 1..36: mol_type = other DNA
cgcggcggcgcgccgaccatgattaccgcgtattat

SEQ ID NO: 27 - DNA, 36 bp, synthetic construct
  source 1..36: mol_type = other DNA
  variation 1: note = m = a or c
  variation 3: note = n = a, c, t, or g
  variation 6: note = n = a, c, g, or t
  variation 9: note = n = a, c, t, or g
  variation 12: note = n = a, c, t, or g
  variation 15: note = n = a, c, t, or g
  variation 18: note = n = a, c, t, or g
  variation 27: note = n = a, c, t, or g
  variation 30: note = n = a, c, t, or g
  variation 24: note = h = a, c or t
  variation 33: note = y = c or t
  variation 36: note = y = c or t
mgnggnggngcnccnacnatgathacngcntaytay

SEQ ID NO: 28 - protein, 448 aa, synthetic construct
EVQLQQSGTVLARPGASVKMSCKASGYSFTSYWMHWVKQRPGQGLEWIGAIYPGNSDTSYNQKFKGKAKLTAVTSASTAYMELSSLTNEDSAVYYCTRGGAPTMITAYYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 29 - protein, 214 aa, synthetic construct
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 30 - protein, 604 aa, Homo sapiens
HVGQDRVDFGQTEPHTVLFHEPGSSSVWVGGRGKVYLFDFPEGKNASVRTVNIGSTKGSCLDKRDCENYITLLERRSEGLLACGTNARHPSCWNLVNGTWPLGEMRGYAPFSPDENSLVLFEGDEVYSTIRKQEYNGKIPRFRRIRGESELYTSDTVMQNPQFIKATIVHQDQAYDDKIYYFFREDNPDKNPEAPLNVSRVAQLCRGDQGGESSLSVSKWNTFLKAMLVCSDAATNKNFNRLQDVFLLPDPSGQWRDTRVYGVFSNPWNYSAVCVYSLGDIDKVFRTSSLKGYHSSLPNPRPGKCLPDQQPIPTETFQVADRHPEVAQRVEPMGPLKTPLFHSKYHYQKVAVHRMQASHGETFHVLYLTTDRGTIHKWEPGEQEHSFAFNIMEIQPFRRAAAIQTMSLDAERRKLYVSSQWEVSQVPLDLCEVYGGGCHGCLMSRDPYCGWDQGRCISIYSSERSVLQSINPAEPHKECPNPKPDKAPLQKVSLAPNSRYYLSCPMESRHATYSWRHKENVEQSCEPGHQSPNCILFIENLTAQQYGHYFCEAQEGSYFREAQHWQLLPEDGIMAEHLLGHACALAASLWLGVLPTLTLGLLVH

(19) United States
(12) Patent Application Publication
(10) Pub. No.: US 2025/0206839 A1
(43) Pub. Date: Jun. 26, 2025

(54) METHODS, COMPOSITIONS AND USES FOR SEMA7A MONOCLONAL ANTIBODIES

(71) Applicant: THE REGENTS OF THE UNIVERSITY OF COLORADO, A BODY CORPORATE, Denver, CO (US)

(72) Inventor: Traci R. Lyons, Denver, CO (US)

(21) Appl. No.: 18/948,222

(22) Filed: Nov. 14, 2024

Related U.S. Application Data
(63) Continuation of application No. PCT/US23/67712, filed on May 31, 2023.
(60) Provisional application No. 63/347,988, filed on Jun. 1, 2022.
(60) Provisional application No. 63/385,370, filed on Nov. 29, 2022.

(51) Int. Cl. C07K 16/28; A61K 39/00; A61P 35/00

(52) U.S. Cl. CPC C07K 16/2896; A61P 35/00; A61K 2039/505; C07K 2317/24; C07K 2317/33

(57) ABSTRACT

Embodiments of the present invention generally relate to monoclonal antibodies or polypeptides directed to bind to SEMA7A. In certain embodiments, these antibodies or polypeptides are of use to treat or reduce onset of health conditions. In certain embodiments, compositions and methods disclosed herein concern using SEMA7A monoclonal antibodies for diagnosis, prevention and/or treatment of cancers, cardiac conditions, and other health conditions. In certain embodiments, SEMA7A levels detected by the monoclonal antibodies disclosed herein can be used to project progression of a condition and course of intervention. In some embodiments, cancers can be targeted. In other embodiments, cancer stem cells can be targeted. In other embodiments, monoclonal antibodies can be used to treat cancers such as breast cancer and other cancers having SEMA7A expression or over-expression in a subject.

### CROSS-REFERENCE TO RELATED APPLICATIONS

This U.S, application is a continuation of International Application No. PCT/US23/67712 filed May 31, 2023, which claims priority to U.S. Provisional Application No. 63/347,988 filed Jun. 1, 2022, and U.S. Provisional Application No. 63/385,370 filed Nov. 29, 2022. These applications and associated documents are incorporated herein by reference in their entireties for all purposes.

### GOVERNMENT FUNDING

This invention was made with government support under grant numbers RO1 CA21696-01A1 and U01HL152405 from the National Institutes of Health. The government has certain rights in the invention.

### FIELD

Embodiments of the present invention generally relate to monoclonal antibodies generated against Semaphorin 7A (SEMA7A) and compositions and methods of making and using these antibodies. In certain embodiments, compositions and methods are disclosed for using these antibodies for diagnosing, reducing the onset, and treating cancer or related disorder.

### SEQUENCE LISTING

The instant application contains a Sequence Listing which has been submitted electronically in XML format and is hereby incorporated by reference in its entirety for all purposes. The XML copy, created on Feb. 18, 2025, is referred to as 106549-829005_CU5954H-05.xml and is 45,056 bytes in size.

### BACKGROUND

Cancer continues to be a leading cause of death worldwide. Breast cancer is the most common cancer in women with about 1.68 million new cases and 520,000 deaths worldwide annually. Of these, estrogen receptor positive breast cancers (ER+BC) are the highest percentage of breast cancers at about 70 percent with a relapse within ten years in about 20 percent of the cases. While some specific therapies have been developed for certain cancers, the distinct physiology and unique characteristics of certain cancers as well as the growing need for cancer treatments have created a need for more specifically targeted preventions or treatments of cancer in a subject.

### SUMMARY

Some embodiments of the present disclosure concern development of anti-Semaphorin 7A (referred to herein as SEMA7A: also referred to as CD108, or Sema KI or Sema L) monoclonal antibodies and their uses in diagnosis, prevention, and/or treatment of health conditions. Certain embodiments concern antibodies and methods of making antibodies thereof, pharmaceutical compositions including the antibodies herein, and methods of administering antibodies and/or compositions to treat health conditions where SEMA7A is produced or over-produced in a subject. In certain embodiments, the health conditions are linked to expression of or over-expression of. SEMA7A. In accordance with these embodiments, these synthetic, isolated antibodies including monoclonal antibodies have a high binding affinity and specificity to SEMA7A. In certain embodiments, these monoclonal antibodies can inhibit SEMA7A expression and/or reduce or eliminate pro-cancer activities. In some embodiments, reducing SEMA7A effects can include, but are not limited to, reducing SEMA7A effects on cell proliferation, expansion, metastasis, migration and/or pro-inflammatory-related side effects, as well as other SEMA7A pro-tumor or pro-inflammatory activities.

In certain embodiments, the present disclosure provides isolated antibodies which can bind to SEMA7A. In some embodiments, isolated SEMA7A antibodies provided herein can include monoclonal antibodies raised against one or more SEMA7A polypeptide fragment having about 75% or more, or up to 100% identity to SEQ ID NO:2 or include at least about 75% up to 100% in identity to amino acids 381-392 of the polypeptide represented by SEQ ID NO: 1. It is contemplated that truncated or mutated versions of these polypeptides can form part of a construct including 75% or more up to 100% identity to SEQ ID NO: 2 or include at least 75% identity to 100% identity to amino acids 381-392 of the polypeptide represented by SEQ ID NO: 1 or isomer thereof. In certain embodiments, the peptide or epitope of SEMA7A for use in generating an antibody against SEMA7A is 100% identical to SEQ ID NO:2 or SEQ ID NO:3 or conjugate or fusion polypeptide thereof.

In some embodiments, an isolated antibody disclosed herein can bind to an epitope of SEMATA, wherein the antibody polypeptide includes a heavy chain variable region having at least 75%: at least 80%; at least 85%, at least 95% up to 100% identity to the amino acid sequence represented by any one of SEQ ID NOs. 5, 9, 10, 11, 22, and 24. In other embodiments, an isolated antibody disclosed herein can bind to an epitope of SEMA7A, wherein the antibody includes a light chain variable region having at least 75%; at least 80%; at least 85%, at least 95% up to 100% identity to the amino acid sequence represented by any one of SEQ ID NOs. 7, 13, 14, 15, and 23. In certain embodiments, an isolated antibody disclosed herein can bind to an epitope of SEMA7A, wherein the antibody includes a heavy chain variable region encoded by a polynucleotide having at least 75%; at least 80%; at least 85%, at least 95% up to 100% identity to the polynucleotide represented by SEQ ID NO. 6. In some embodiments, an isolated antibody disclosed herein can bind to an epitope of SEMA7A, wherein the antibody includes a light chain variable region encoded by a polynucleotide having at least 75%; at least 80%; at least 85%, at least 95% up to 100% identity to the polynucleotide represented by SEQ ID NO. 8. In some embodiments, an isolated antibody disclosed herein can bind to an epitope of SEMA7A and reduce or eliminate adverse SEMA7A activities (e.g., pro-tumor activities): the antibody can include a light chain variable region encoded by a polynucleotide having at least 75%; at least 80%; at least 85%, at least 95% up to 100% identity to the polynucleotide represented by SEQ ID NO. 17. In other embodiments, an isolated antibody disclosed herein can bind to an epitope of SEMATA and reduce or eliminate adverse SEMA7A activities (e.g., pro-tumor activities): the antibody can include a heavy chain variable region encoded by a polynucleotide encoding the polypeptide represented by the polypeptide having at least 75%; at least 80%; at least 85%, at least 95% up to 100% identity to the polypeptide sequence represented by any one of SEQ ID NOs. 5, 9, 10, 11, 22 and 24. In certain embodiments, an isolated antibody disclosed herein can bind to an epitope of SEMA7A and reduce or eliminate adverse SEMA7A activities (e.g., pro-tumor activities); the antibody can include a light chain variable region encoded by a polynucleotide encoding the polypeptide represented by the polypeptide having at least 75%; at least 80%: at least 85%, at least 95% up to 100% identity to the polypeptide sequence represented by any one of SEQ ID NOs. 7, 13, 14, 15, and 23. In certain embodiments, an encoded light chain variable region of the antibody and the encoded heavy chain variable region of the antibody that can bind to an epitope of SEMA7A and reduce or eliminate adverse SEMA7A activities (e.g., pro-tumor activities) can encode a polypeptide represented by any combination of these sequences that encode a light chain variable region of the antibody and encode a heavy chain variable region of the antibody to generate a single antibody that can bind to an epitope of SEMA7A and reduce or eliminate adverse SEMA7A activities (e.g., pro-tumor activities, pro-inflammatory activities). In certain embodiments, the antibody can be a full-length antibody or a fragment thereof, or fusion polypeptide thereof.

In some embodiments, an isolated antibody disclosed herein can include a heavy chain complementary determining region 3 (HC CDR3) having at least 75%: at least 80%; at least 85%, at least 95%, up to 100% identity to the polypeptide sequence represented by SEQ ID NO. 11. In some embodiments, an isolated antibody disclosed herein can include a light chain complementary determining region 3 (LC CDR3) having at least 75%: at least 80%; at least 85%, at least 95%, up to 100% identity to the polypeptide sequence represented by SEQ ID NO: 15. In some embodiments, an isolated antibody disclosed herein can include a heavy chain complementary determining region 3 (HC CDR3) having at least 75%; at least 80%: at least 85%, at least 95%, up to 100% identity to the polypeptide sequence represented by SEQ ID NO. 11; and a light chain complementary determining region 3 (LC CDR3) having at least 75%: at least 80%; at least 85%, at least 95%, up to 100% identity to the polypeptide sequence represented by SEQ ID NO: 15. In certain embodiments, a CDR region disclosed herein can bind to an epitope of SEMA7A and reduce or eliminate adverse SEMA7A activities (e.g., pro-tumor activities, pro-inflammatory activities). In other embodiments, the antibody can include a polypeptide represented by any combination of these sequences of a light chain variable region of the antibody and a heavy chain variable region of the antibody to generate a single antibody that can bind to an epitope of SEMATA and reduce or eliminate adverse SEMA7A activities (e.g., pro-tumor activities). In certain embodiments, the antibody can be a full-length antibody or a fragment thereof or fusion polypeptide thereof.

In some embodiments, an isolated antibody disclosed herein can bind to an epitope of SEMATA and reduce or eliminate adverse SEMA7A activities (e.g., pro-tumor activities); the antibody polypeptide can include a heavy chain variable region represented by an amino acid sequence represented by any one of the polypeptides represented by any one of SEQ ID NOs. 5, 9, 10, and 11. In other embodiments, an isolated antibody disclosed herein can bind to an epitope of SEMA7A and reduce or eliminate adverse SEMA7A activities (e.g., pro-tumor activities); the antibody polypeptide can include a light chain variable region represented by any one of the polypeptides represented by any one of SEQ ID NOs. 7, 13, 14, and 15. In certain embodiments, an isolated antibody disclosed herein can bind to an epitope of SEMATA, where the antibody includes a heavy chain variable region encoded by a polynucleotide represented by SEQ ID NO. 6. In some embodiments, an isolated antibody disclosed herein can bind to an epitope of SEMA7A, where the antibody includes a light chain variable region encoded by a polynucleotide represented by SEQ ID NO. 8. In other embodiments, an isolated antibody disclosed herein can bind to an epitope of SEMA7A, where the antibody includes a heavy chain variable region encoded by a polynucleotide encoding the polypeptide represented by any one of SEQ ID NOs. 5, 9, 10, 11, 22 and 24. In certain embodiments, an isolated antibody disclosed herein can bind to an epitope of SEMA7A, where the antibody includes a light chain variable region encoded by a polynucleotide encoding the polypeptide represented by any one of SEQ ID NOs. 7, 13, 14, 15, and 23. In certain embodiments, a light chain variable region of the antibody and a heavy chain variable region of the antibody that can bind to an epitope of SEMA7A and reduce or eliminate adverse SEMA7A activities (e.g., pro-tumor activities) can include a polypeptide represented by any combination of these sequences that of a light chain variable region of the antibody and a heavy chain variable region of the antibody to generate a single antibody that can bind to an epitope of SEMA7A and reduce or eliminate adverse SEMA7A activities (e.g., pro-tumor activities). In certain embodiments, the antibody can be a full-length antibody or a fragment thereof or fusion polypeptide thereof.

In some embodiments, an isolated antibody disclosed herein can include a heavy chain complementary determining region 3 (HC CDR3) represented by a polypeptide of SEQ ID NO: 11. In some embodiments, an isolated antibody disclosed herein can include a light chain complementary determining region 3 (LC CDR3) represented by a polypeptide represented by SEQ ID NO: 15. In some embodiments, an isolated antibody disclosed herein can include a heavy chain complementary determining region 3 (HC CDR3) represented by the polypeptide sequence represented by SEQ ID NOs: 11 or 24; and a light chain complementary determining region 3 (LC CDR3) represented by the polypeptide sequence represented by SEQ ID NO: 15. In some embodiments, an isolated antibody disclosed herein can include a heavy chain complementary determining region 3 (HC CDR3) represented the polypeptide represented by SEQ ID NO: 11 and a light chain complementary determining region 3 (LC CDR3) represented by the polypeptide sequence represented by SEQ ID NO: 15. In some embodiments, an isolated antibody disclosed herein can include a heavy chain complementary determining region 3 (HC CDR3) represented the polypeptide represented by SEQ ID NO: 24 and a light chain complementary determining region 3 (LC CDR3) represented by the polypeptide sequence represented by SEQ ID NO: 15. In certain embodiments, a CDR region disclosed herein can bind to an epitope of SEMA7A and reduce or eliminate adverse SEMA7A activities (e.g., pro-tumor activities). In other embodiments, the antibody can include a polypeptide represented by any combination of these sequences of a light chain variable region of the antibody and a heavy chain variable region of the antibody to generate a single antibody that can bind to an epitope of SEMA7A and reduce or eliminate adverse SEMA7A activities (e.g., pro-tumor activities). In certain embodiments, the antibody can be a full-length antibody or a fragment thereof or fusion polypeptide thereof.

In some embodiments, an isolated antibody disclosed herein can be a full-length antibody or an antigen-binding fragment thereof (e.g.,. SEMA7A epitope). In some embodiments, an isolated antibody disclosed herein can be a full-length antibody, such as an IgG molecule. In some embodiments, an isolated antibody disclosed herein can be an antibody fragment and can be a Fab, a (Fab) 2, and/or a single-chain antibody able to bind to an antigen or epitope of SEMA7A. In some embodiments, an isolated antibody disclosed herein can be a human antibody or a humanized antibody.

In some embodiments, an isolated antibody disclosed herein can include a polynucleotide encoding a heavy chain variable region and a polynucleotide encoding a light chain variable region located on one or two vectors. In accordance with these embodiments, the one or two vectors can include one or two expression vectors for expressing the heavy and/or light chain directed to bind SEMA7A.

In some embodiments, the present disclosure provides isolated antibodies, which bind to SEMA7A with high specificity and reduce or eliminate SEMA7A activities. In certain embodiments. SEMA7A activities include, but are not limited to, tumor promoting activities such as expansion, metastasis, and migration of tumor cells. In certain embodiments, the tumor cells include breast, pancreatic, brain, liver cancer or leukemia. In some embodiments, monoclonal antibodies disclosed herein can include monoclonal antibodies directed to bind SEQ ID NO:2, SEQ ID NO:3 or conjugate or fragment thereof (e.g., referenced as clone H1 or H1).

In certain embodiments, the present disclosure includes host cells of use in generating antibodies contemplated herein. In some embodiments, a host cell can include a polynucleotide encoding at least one of the heavy chain variable regions disclosed herein and/or a polynucleotide encoding at least one of the light chain variable regions disclosed herein. In some embodiments, a heavy chain variable region can include a polynucleotide encoding the polypeptide of one or more polypeptide sequence represented by SEQ ID NOs. 5, 9, 10, 11, 22 and 24. In certain embodiments, a light chain variable region can include a polynucleotide encoding the polypeptide of one or more polypeptide sequence represented by SEQ ID NOs. 7, 13, 14, 15, and 23.

In some embodiments, a pharmaceutical composition disclosed herein can include a heavy chain variable region represented by an amino acid sequence represented by the polypeptides represented by any one of SEQ ID NOs. 5, 9, 10, and Iland a pharmaceutically acceptable excipient. In certain embodiments, a pharmaceutical composition disclosed herein can include a heavy chain variable region represented by an amino acid sequence represented by the polypeptides represented by any one of SEQ ID NOs. 11 and a pharmaceutically acceptable excipient.

In certain embodiments, methods of treating a subject having a health condition where SEMA7A is expressed or overexpressed are contemplated. In some embodiments, the health condition includes cancer, a heart condition, or an inflammatory condition. In certain embodiments, the health condition is breast cancer. In some embodiments, methods for treating the health conditions disclosed herein include, but are not limited to, administering to a subject in need thereof an effective amount of a pharmaceutical composition containing a full-length monoclonal antibody or fragment thereof disclosed herein.

In some embodiments, methods disclosed herein include, but are not limited to treating cancer in a subject. In accordance with these embodiments, compositions disclosed herein can be used to reduce or eliminate SEMA7A-related tumor cell metastasis, migration, expansion, or tumor volume by blocking or inhibiting these SEMA7A activities using SEMA7A antibody or antibody fragment formulations. In certain embodiments, compositions disclosed herein can reduce or eliminate cancer stem cells compared to other treatments and/or compared to using other SEMA7A antibodies. In other embodiments, compositions disclosed herein can be used to diagnose breast cancer, breast cancer relapse, survivability, and/or response to standard anti-cancer treatments by obtaining one or more samples from a subject and exposing the one or more samples to a SEMANA antibody or antibody fragment formulation disclosed herein (e.g., H1 antibody or fragment thereof-containing composition).

In certain embodiments, antibodies (e.g., monoclonal antibodies) disclosed herein directed to bind SEMA7A can be used to treat health conditions associated with this molecule. In other embodiments, antibodies (e.g., monoclonal antibodies) disclosed herein directed to bind SEMA7A can be used to diagnose a health conditions associated with this molecule. In some embodiments, concentrations of SEMA7A in a sample from a subject can be assessed. In other embodiments, changes in concentrations of SEMA7A in samples from a subject can be assessed to monitor treatment regimens for further adjustment to a treatment regimen or to combine antibody treatments disclosed herein with known methods to treat the condition (e.g., cancer).

In some embodiments, cancer can be treated in a subject contemplated herein with SEMA7A antibodies generated against SEQ ID NO:2 or SEQ ID NO:3 or conjugate thereof disclosed herein (e.g., H1) to reduce onset, ameliorate or eliminate the cancer. In some embodiments, antibodies to SEMA7A (e.g., H1) disclosed herein can reduce or eliminate SEMA7A activity such as tumor progression, reduce or inhibit metastasis, shrink tumors, induce tumor cell death, reduce tumor expansion, reduce lymphangiogenesis/angiogenesis lymphovascular invasion and/or reduce lymphogenous/hematogenous metastasis (e.g., reduce infiltration or metastasis of tumor cells into lymph nodes or distant organs)

In certain embodiments, the isolated SEMA7A antibody or monoclonal antibody is a full-length antibody or SEMA7A binding fragment thereof, such as a heavy chain region (e.g., SEQ ID NO: 5, 6, 9, 10, or 11). In some embodiments, the isolated anti-SEMA7A antibody is a full-length monoclonal antibody as reference herein above and throughout this disclosure. In certain embodiments, the isolated anti-SEMA7A antibody is a single-chain antibody (scFv).

In other embodiments, a polynucleotide encoding the SEMA7A antibody, monoclonal antibody or antigen binding fragment thereof provided herein is contemplated. In accordance with these embodiments, the polynucleotide can further include a vector for expressing the encoded antibody or antigen binding fragment. In vet other embodiments, host cells having the polynucleotide encoding a SEMA7A antibody, monoclonal antibody or antigen binding fragment thereof are included herein. In certain embodiments, particular peptides are contemplated of use herein to target SEMA7A and SEMA7A activities (e.g., pro-tumor activities). In some embodiments, a peptide or immunopeptide or antibody having or containing a fragment having at least 80% amino acid sequence identity to the sequence of the amino acid sequence represented by SEQ ID NO. 24. RGGAPTMITAYY is contemplated. In some embodiments, the immunopeptide is a full-length antibody or fragment thereof having at least 80% or at least 90% or about 100% amino acid sequence identity to the sequence of the amino acid sequence represented by SEQ ID NO. 24. RGGAPTMITAYY. In certain embodiments, the polypeptide is part of a larger polypeptide such as an immunoglobulin for example, the polypeptide can be part of an antibody (e.g., monoclonal antibody) or fragment of an antibody having biological activity. In some embodiments, the polypeptide can be about 5 amino acids to about 12 amino acids in length having 100% identity to a contiguous portion of SEQ ID NO. 24. RGGAPTMITAYY. In other embodiments, the polypeptide can be about 5 amino acids to about 1.000 amino acids in length, or about 10 to about 750 amino acids in length, or about 10 to about 650 amino acids in length, or about 10 to about 600 amino acids in length or about 10 to about 550 amino acids in length or about 50 to about 500 amino acids in length having about 80%, or about 85%, or about 90% or about 95% or up to 100% or about 80% up to about 100%, sequence identity to a contiguous portion of SEQ ID NO. 24. RGGAPTMITAYY. In certain embodiments, the peptide includes a fragment having at least 90% sequence identity to the polypeptide sequence represented by SEQ ID NO. 24. RGGAPTMITAYY. In other embodiments, the peptide includes a fragment having 100% identity in a larger polypeptide than the fragment (e.g., fusion peptide, antibody or peptide conjugate or chimera) directed to bind to SEMA7A. In accordance with these embodiments, the polypeptide having about 80% to about 100% amino acid sequence identity to the polypeptide sequence represented by SEQ ID NO. 24. RGGAPTMITAYY reduces, essentially blocks or completely blocks SEMA7A activity and function. In certain embodiments, these peptide constructs or antibody or monoclonal antibody having about 80% to about 100% amino acid sequence identity to the polypeptide sequence represented by SEQ ID NO. 24. RGGAPTMITAYY inhibits, or essentially blocks, or blocks SEMA7A pro-tumor activity or pro-inflammatory activity. In some embodiments, the peptide can form part of a composition further including at least one of a chemotherapeutic or anti-tumor agent or complex thereof. In certain embodiments, these peptides can form part of a pharmaceutical composition of use to treat a condition having aberrant SEMA7A activity (e.g., a condition characterized in SEMA7A expression or overexpression).

In some embodiments and further to the paragraphs above, methods can include administering a pharmaceutically acceptable composition disclosed herein and treating a health condition characterized in SEMA7A expression or overexpression in a subject in need thereof. In some embodiments and further to the paragraphs above, methods can include administering a pharmaceutically acceptable composition disclosed herein and inducing anti-tumor activities in the subject in need thereof. In accordance with these embodiments. pharmaceutical compositions described herein can be administered to the subject by any method known in the art for administering an antibody, monoclonal antibody, or fragment thereof. In another embodiment, methods are provided for diagnosing and/or treating cancer or other health condition having overproduction or production at a predetermined concentration of SEMA7A in the subject.

In some embodiments, a condition characterized in SEMA7A expression or overexpression treatable by compositions containing monoclonal antibodies disclosed herein include, but are not limited to, cancer, an inflammatory condition, a cardiac condition, a fibrotic condition, or a combination thereof. In some embodiments, a health condition treatable by compositions containing monoclonal antibodies disclosed herein include, but are not limited to, arthritis, colitis, inflammatory bowel disease, airway inflammation. Gaucher disease, myocardial infarction, liver fibrosis, lung fibrosis or a combination thereof. In certain embodiments, when treating any condition disclosed herein, the pharmaceutical compositions can be administered systemically, topically, by bolus infusion, intranasally, intravenously. subcutaneously, by slow-release, by seed implant, orally, or by direct local administration into an affected tissue of the subject. In some embodiments, cancers to be treated by compositions containing monoclonal antibodies disclosed herein include a solid tumor. In other embodiments, the solid tumor includes, but is not limited to, breast, prostate, liver, lung, kidney, stomach, ovarian, head, neck, brain, skin, testicular, pancreatic or another solid tumor. In certain embodiments, when treating solid tumors, the pharmaceutical compositions can be administered systemically, topically, by bolus infusion, intranasally, intravenously, subcutaneously, by slow-release, by seed implant, orally, or by direct local administration into a solid tumor in the subject.

In some embodiments, compositions, and methods for inhibiting SEMA7A expression and/or activity disclosed herein can be used to reduce or prevent expansion, migration, metastasis and/or maturation of cancer stem cells. In certain embodiments, the cancer stem cells include any cancer stem cell of any origin. In other embodiments, the cancer stem cells include any cancer stem cell resistant to radiation, chemotherapy, or other anti-cancer treatment where compositions and methods disclosed herein can be used to reduce or prevent cancer stem cell activities and survival to fully treat a subject in need thereof.

In certain embodiments, kits are contemplated for use in storing, transporting and using monoclonal antibodies or fragments thereof (e.g., isolated monoclonal antibodies, pharmaceutical compositions, host cells) disclosed herein. In some embodiments, kits are contemplated for practicing any of the methods disclosed herein (e.g., treating/inhibiting a subject having a condition expressing or overexpressing SEMA7A, generating isolated antibodies, generating pharmaceutical compositions, and/or generating host cells) disclosed herein.

### BRIEF DESCRIPTION OF THE DRAWINGS

The following drawings form part of the present specification and are included to further demonstrate certain embodiments of the present disclosure. Certain embodiments can be better understood by reference to one or more of these drawings in combination with the detailed description of specific embodiments presented herein.

FIGS. 1A-1B represent (1A) a schematic diagram illustrating a protein structure of SEMA7A and (1B) a representative SEMA7A sequence with the epitope of interest bolded and underlined and a schematic of the protein domains according to various aspects of the disclosure.

FIGS. 2A-2B represent an exemplary (2A) Western blot depicting recognition of all three isoforms of SEMATA by mouse monoclonal antibody generated using an immunizing peptide (e.g., 10-mer) and termed SmAbH1 (right panel). Balb/c mice were inoculated with mouse mammary tumors followed by monoclonal antibody treatments for SEMA7A (SmAbH1): (2B) mice were inoculated with mouse mammary tumor cells and later treated with SmAbH1. (2C) mice were inoculated with mouse mammary tumor cells and later treated with a SEMA7A monoclonal antibody disclosed herein and studied for effect according to various aspects of the disclosure.

FIGS. 3A-3B illustrates representative survival curves: (3A) survival curves for mice treated with monoclonal antibodies against SmAbHIfrom FIG. 7B; and (3B) survival analysis for time to tumor size of >200 mm3 in mice treated with SmAbH1, or IgG control, from FIG. 7C according to various aspects of the disclosure.

FIGS. 4A-4D are graphical representations of mice from FIG. 2C with respect to immune cell activation. (4A) represents analysis of percent immune cells within a representative tumor using flow cytometry for commonly observed immune cell-associate leukocyte antigen (CD45): (4B) represents an analysis of percent CD4+ T cells of CD45+immune (CD45+B220-CD3+CD8-) cells observed using flow cytometry: (4C) represents analysis of precent CD8+ T cells of CD45+immune (CD45+B220-CD3+CD4-) cells using flow cytometry; and (4D) represents analysis of activated T cells, based on expression of interferon gamma (IFNg) and tumor necrosis factor alpha (TNFa) on CD8+ T cells by flow cytometry. * p<0.05. ** p<0.01, t-test according to various aspects of the disclosure.

FIGS. 5A-5E represents graphical illustrations of exemplary data for (5A) survival statistics for time to tumor size to >500 mm3 in mice injected with tumors and treated with a commercially available anti-tumor antibody (e.g., anti-PD-L1, anti-PD-1). SEMA7A monoclonal antibody monotherapy or a combination therapy: (5B) represents exemplary tumor growth curves when tumor size is about 150 mm3 treated with a commercially available anti-tumor antibody (e.g., anti-PD-L1, anti-PD-1). SEMA7A monoclonal antibody monotherapy or a combination therapy (black arrows indicate treatments): (5C) illustrates individual tumor volumes from each group pre-treatment and at study end: (5D) illustrates percent of mice with evidence of lung or lymph node metastasis in each treatment group; and (5E) mouse mammary tumor growth in mice treated with a commercially available anti-tumor antibody monotherapy starting after tumor establishment in the mice where percent of mice with metastasis at the study end is represented to the right of each experimental plot according to various aspects of the disclosure.

FIGS. 6A-6B illustrate in (6A) that monoclonal antibodies to SEMA7A generated from a peptide disclosed herein reduces mammosphere formation; and (6B) cell death in vitro (blue=control above the solid grey plot and red=SEMA7A OE above the clear plots) according to various aspects of the disclosure.

FIGS. 7A-7B illustrates effects of control versus SEMA7A monoclonal antibodies versus control on tumor volume (7A) and invasion scores (7B) by staining analysis on harvested tissues from 7A according to various aspects of the disclosure.

FIGS. 8A-8C illustrates cell viability in ER+ (8A) human derived Michigan Cancer Foundation 7 (MCF7): (8B) mouse derived TC11 and (8C) mouse derived SSM2 cells (selected for responsiveness to estrogen and to inhibitors of estrogen receptor) were treated for 48 hours with either IgG control, an estrogen receptor antagonist (e.g., fulvestrant (Fulv)). SEMA7A monoclonal antibody (e.g., . SmAb H1) or the combination thereof according to various aspects of the disclosure.

FIG. 9 illustrates a studied parameter of pro-tumor cells, percent confluence in cell culture of tumor cells (e.g., 66cl4). This figure illustrates a time course study of confluence over time in the presence of control or increasing concentrations of SEMA7A monoclonal antibody (mAb. SmAbH1) demonstrating dose dependency where reduced levels of SEMA7A affected confluence according to various aspects of the disclosure.

FIGS. 10A-10C illustrates percent confluence of control (mouse derived 66cl4 DDK) and SEMA7A OE (DDK SEMA7A) cells treated with (10A) SEMA7A monoclonal antibody (mAb. SmAbH1). (10B) commercially available SEMA7A clone (Santa Cruz. D4. #376149): (Santa Cruz. D4. Catalog #sc-376149; immunizing peptide represented by SEQ ID NO:4. QPIPTETFQVADRHPEVAQRVEPMGPLKTPLFHSKYHYQKV); and (10C) another SEMA7A polyclonal antibody (Ab23578: immunizing peptide within residues 1-100) according to various aspects of the disclosure. Based on previous observations it is likely that the decreased confluence is due to antibody blocking the SEMA7A promoting effects on cell growth, cell survival and stem cell phenotypes

FIGS. 11A-11B represent an exemplary (11A) Western blot demonstrating presence or absence of SEMA7A binding by an exemplary antibody (e.g., . SmAbH1 CUH1) in control cell lysates (empty vector-EV) or engineered cells expressing a tagged version of SEMA7A where the endogenous SEMA7A is 75 kD and the tagged version is 100 kD and (11B) an exemplary dose-response curve from an Enzyme Linked Immunosorbent Assay (ELISA) demonstrating binding of an antibody disclosed herein to SEMA7A in a dose dependent manner according to various embodiments of the disclosure.

FIG. 12 illustrates a representative image of an enzyme linked immunosorbent assay to detect a recombinant antibody's ability to bind to SEMA7A in the presence and absence of the immunizing peptide (10×SEMA7 peptide), as measured by absorbance at 425 nm according to various embodiments of the disclosure.

FIG. 13 illustrates a representative immunofluorescence staining image using an antibody of human breast cancer (MCF7) SEMA7A overexpressing (OE) cells treated with an exemplary TACE/ADAM17 inhibitor according to various embodiments of the disclosure.

FIGS. 14A-14B illustrates immunoblot analysis of a human breast cancer cell line having low (MDA468, 14A) or high (MDA231, 14B) expression levels of SEMA7A treated with an antibody or monoclonal antibody-based inhibitors of a known integrin receptor for SEMA7A, a6b1, according to various embodiments of the disclosure.

FIGS. 15A-15B illustrates an immunoblot analysis of human breast cancer cell lines having low (MDA468, 15A) or high (MDA231, 15B) expression of SEMA7A treated with an antibody disclosed herein or monoclonal antibody-based inhibitors of a known integrin receptor for SEMA7A, a6b1, according to various embodiments of the disclosure.

### Definitions

As used herein, the term “about,” can mean relative to the recited value, e.g., amount, dose, temperature, time, percentage, etc., ±10%, ±9%, ±8%, ±7%, ±6%, ±5%, ±4%, ±3%, ±2%, or ±1%.

As used herein, the terms “treat,” “treating,” “treatment” and the like, unless otherwise indicated, can refer to reversing, alleviating, inhibiting the process of or expansion of, or preventing the disease, disorder or condition to which such term applies, or one or more symptoms of such disease, disorder or condition and includes the administration of any of the compositions, pharmaceutical compositions, or dosage forms described herein, to prevent the onset of the symptoms or the complications, or alleviating the symptoms or the complications, or eliminating the condition, or disorder.

An “epitope” as used herein can refer to the site on a target antigen that is recognized and bound by an antibody. In some embodiments, the site can be entirely composed of amino acid components, entirely composed of chemical modifications of amino acids of the protein (e.g., glycosyl moieties), or composed of combinations thereof. In some embodiments, overlapping epitopes can include at least one common amino acid residue. In some embodiments, two or more SEMA7A antibodies or monoclonal antibodies or epitope-binding fragment thereof described herein can bind to the same epitope or a substantially overlapping epitope (e.g., containing less than 3 non-overlapping amino acid residues, less than 2 non-overlapping amino acid residues, or only 1 non-overlapping amino acid residue).

Certain constructs and polypeptides are described below in terms of “percent identity” or “percent sequence identity” or “percent homology” or “percent sequence homology” to a reference sequence. When used herein, the term “percent identity” or “percent sequence identity” or “percent homology” of two amino acid sequences can be determined by any method known in the art.

Constructs and polypeptides having a certain percent sequence homology to a reference sequence can have one or more “conservative amino acid substitutions” relative to the reference sequence. These “conservative amino acid substitution(s)” can refer to one or more amino acid substitution that does not alter the relative charge or size characteristics of the protein in which the amino acid substitution is made. In some embodiments, variants herein can be prepared according to methods for altering polypeptide sequence known to one of ordinary skill in the art.

### DETAILED DESCRIPTION

In the following sections, certain exemplary compositions and methods are described in order to detail certain embodiments of the invention. It will be obvious to one skilled in the art that practicing the certain embodiments does not require the employment of all or even some of the specific details outlined herein, but rather that concentrations, times, and other specific details can be modified through routine experimentation. In some cases, well known methods, or components have not been included in the description.

Some embodiments of the present disclosure are based on development of anti-SEMA7A monoclonal antibodies and their uses in the diagnosis, reducing the risk of onset and/or treating a health conditions. Certain embodiments concern antibodies and methods of making antibodies thereof, pharmaceutical compositions including the antibodies herein, and methods of administering antibodies and/or compositions to treat health conditions where SEMA7A is produced or over-produced in a subject. In certain embodiments, the health conditions are linked to expression of or over-expression of, SEMA7A. In accordance with these embodiments, these antibodies including monoclonal antibodies have a high binding affinity and specificity to SEMATA and can inhibit SEMA7A expression and/or pro-cancer activity or other activities and reduce SEMA7A effects on cell proliferation, expansion, metastasis, migration and/or pro-inflammatory-related side effects, as well as other SEMA7A pro-tumor activities.

In certain embodiments and further to paragraph above, the present disclosure provides isolated antibodies which can bind to SEMA7A. In some embodiments. an isolated SEMA7A antibody provided herein can include monoclonal antibodies raised against one or more SEMA7A polypeptide fragment having about 75%, or about 85%, or about 90%, or about 95%, or up to 100% identity to SEQ ID NO:2 or include at least about 75% up to 100% in identity to amino acids 381-392 of the polypeptide represented by SEQ ID NO: 1. It is contemplated that truncated or mutated versions of these polypeptides can form part of a construct including 75% or more up to 100% identity to SEQ ID NO:2 or include at least 75% identity to 100% identity to amino acids 381-392 of the polypeptide represented by SEQ ID NO: 1 or isomer thereof. In certain embodiments, the peptide or epitope of SEMATA of use to generate an antibody against SEMA7A is 100% identical to SEQ ID NO:2 or SEQ ID NO:3 or conjugate or fusion polypeptide thereof.

In some embodiments and further to paragraphs [0049]-[0050] above, an isolated antibody disclosed herein can bind to an epitope of SEMA7A, wherein the antibody polypeptide includes a heavy chain variable region having at least 75%; at least 80%; at least 85%, at least 90%, at least 95% up to 100% identity to the amino acid sequence represented by any one of SEQ ID NOs. 5, 9, 10, 11, 22 and 24. In other embodiments, an isolated antibody disclosed herein can bind to an epitope of SEMA7A, wherein the antibody includes a light chain variable region having at least 75% up to about 100%; at least 80%: at least 85%, at least 90%, at least 95%, up to 100% identity to the amino acid sequence represented by any one of SEQ ID NOs. 7, 13, 14, 15, and 23. In certain embodiments, an isolated antibody disclosed herein can bind to an epitope of SEMA7A, wherein the antibody includes a heavy chain variable region encoded by a polynucleotide having at least 75%; at least 80%: at least 85%, at least 90%, at least 95% up to 100% identity to the polynucleotide represented by SEQ ID NO. 6. In certain embodiments, an isolated antibody disclosed herein can bind to an epitope of SEMA7A and reduce or eliminate SEMA7A adverse activities (e.g., pro-tumor activities), where the antibody includes a heavy chain variable region encoded by a polynucleotide having at least 75%; at least 80%; at least 85%, at least 90%, at least 95% up to 100% identity to the polynucleotide represented by SEQ ID NO. 25, 26 or 27. In some embodiments, an isolated antibody disclosed herein can bind to an epitope of SEMA7A and reduce or eliminate SEMA7A adverse activities (e.g., pro-tumor activities), where the antibody includes a light chain variable region encoded by a polynucleotide having at least 75% up to about 100%; at least 80%: at least 85%, at least 95% up to 100% identity to the polynucleotide represented by SEQ ID NO. 8. In other embodiments, an isolated antibody disclosed herein can bind to an epitope of SEMA7A and reduce or eliminate SEMA7A adverse activities (e.g., pro-tumor activities, pro-inflammatory activities), where the antibody includes a heavy chain variable region encoded by a polynucleotide encoding the polypeptide represented by the polypeptide having at least 75% up to about 100%: at least 80%; at least 85%, at least 90%, at least 95% up to 100% identity to the polypeptide sequence represented by any one of SEQ ID NOs. 5, 9, 10, 11, 22 and 24. In certain embodiments, an isolated antibody disclosed herein can bind to an epitope of SEMA7A and reduce or eliminate SEMA7A adverse activities (e.g., pro-tumor activities), where the antibody includes a light chain variable region encoded by a polynucleotide encoding the polypeptide represented by the polypeptide having at least 75%; at least 80%; at least 85%, at least 90%, at least 95% up to 100% identity to the polypeptide sequence represented by any one of SEQ ID NOs. 13, 14. 15, and 23.

In some embodiments and further to paragraphs [0049]-[0051] above, an isolated antibody disclosed herein can include a heavy chain complementary determining region 3 (HC CDR3) having at least 75%; at least 80%; at least 85%, at least 90%, at least 95% up to 100% identity to the polypeptide sequence represented by any one of SEQ ID NOs: 11, and 24. In some embodiments, an isolated antibody disclosed herein can include a light chain complementary determining region 3 (LC CDR3) having at least 75%: at least 80%: at least 85%, at least 90%, at least 95% up to 100% identity to the polypeptide sequence represented by SEQ ID NO: 15. In some embodiments, an isolated antibody disclosed herein can include a heavy chain complementary determining region 3 (HC CDR3) having at least 75%: at least 80%: at least 85%, at least 90%, at least 95% up to 100% identity to the polypeptide sequence represented by SEQ ID NOs: 11 or 24; and a light chain complementary determining region 3 (LC CDR3) having at least 75%; at least 80%; at least 85%, at least 90%, at least 95%, up to 100% identity to the polypeptide sequence represented by SEQ ID NO: 15.

In certain embodiments and further to paragraphs [0049]-[0052] above, the present disclosure provides isolated monoclonal antibodies which can bind to SEMA7A with improved treatment outcomes compared to commercially available antibodies. In some embodiments, an isolated SEMA7A antibody provided herein can include monoclonal antibodies raised against SEQ ID NO:2 or 3, or amino acids 381-392 of the polypeptide represented by SEQ ID NO:1. In certain embodiments, the peptide or epitope of SEMA7A of use to generate an antibody against S.EMA7A is SEQ ID NO:2 or SEQ ID NO:3 or conjugate or fusion polypeptide thereof. It is contemplated herein that any of the antibodies or antibody fragments disclosed herein that bind to SEMA7A can be part of a conjugate or fusion polypeptide that includes multiple repeats of the polypeptide or fusion to at least one other polypeptides.

In some embodiments and further to paragraphs [0049]-[0053] above, an isolated antibody disclosed herein can bind to an epitope of SEMA7A and reduce or eliminate SEMA7A adverse activities (e.g., pro-tumor activities), where the antibody polypeptide includes a heavy chain variable region represented by an amino acid sequence represented by any one of the polypeptides represented by any one of SEQ ID NOs. 5, 9, 10, 11, 22 and 24. In other embodiments, an isolated antibody disclosed herein can bind to an epitope of SEMA7A and reduce or eliminate SEMA7A adverse activities (e.g., pro-tumor activities). where the antibody includes a light chain variable region represented by any one of the polypeptide sequences represented by SEQ ID NOs. 7, 13, 14, 15, and 23. In certain embodiments, an isolated antibody disclosed herein can bind to an epitope of SEMA7A and reduce or eliminate SEMA7A adverse activities (e.g., pro-tumor activities), where the antibody includes a heavy chain variable region encoded by a polynucleotide represented by SEQ ID NO. 6. In certain embodiments, an isolated antibody disclosed herein can bind to an epitope of SEMA7A and reduce or eliminate SEMA7A adverse activities (e.g., pro-tumor activities), where the antibody includes a heavy chain variable region represented by SEQ ID NO. 25, 26 or 27. In some embodiments, an isolated antibody disclosed herein can bind to an epitope of SEMA7A and reduce or eliminate SEMA7A adverse activities (e.g., pro-tumor activities), where the antibody includes a light chain variable region encoded by a polynucleotide represented by SEQ ID NO. 8. In other embodiments, an isolated antibody disclosed herein can bind to an epitope of SEMA7A and reduce or eliminate SEMA7A adverse activities (e.g., pro-tumor activities), where the antibody includes a heavy chain variable region encoded by a polynucleotide encoding the polypeptide represented by any one of SEQ ID NOs. 5, 9, 10, 11, 22 and 24. In certain embodiments, an isolated antibody disclosed herein can bind to an epitope of SEMA7A and reduce or eliminate SEMA7A adverse activities (e.g., pro-tumor activities), where the antibody includes a light chain variable region encoded by a polynucleotide encoding the polypeptide sequence represented by any one of SEQ ID NOs. 7, 13, 14, 15, and 23.

In some embodiments and further to paragraphs [0049]-[0054] above, an isolated antibody disclosed herein can include a heavy chain complementary determining region 3 (HC CDR3) represented by a polypeptide of SEQ ID NO: 11. In some embodiments, an isolated antibody disclosed herein can include a light chain complementary determining region 3 (LC CDR3) represented by a polypeptide represented by SEQ ID NO: 15. In some embodiments, an isolated antibody disclosed herein can include a heavy chain complementary determining region 3 (HC CDR3) represented by the polypeptide sequence represented by SEQ ID NOs: 11 or 24; and a light chain complementary determining region 3 (LC CDR3) represented by the polypeptide sequence represented by SEQ ID NO: 15. In some embodiments, an isolated antibody disclosed herein can include a heavy chain complementary determining region 3 (HC CDR3) represented the polypeptide represented by SEQ ID NO: 11 and a light chain complementary determining region 3 (LC CDR3) represented by the polypeptide sequence represented by SEQ ID NO: 15. In some embodiments, an isolated antibody disclosed herein can include a heavy chain complementary determining region 3 (HC CDR3) represented the polypeptide represented by SEQ ID NO: 24 and a light chain complementary determining region 3 (LC CDR3) represented by the polypeptide sequence represented by SEQ ID NO: 15. In accordance with these embodiments, any of the referenced isolated antibodies, full-length or fragments thereof can be part of a pharmaceutical composition of use to treat a subject disclosed herein (e.g., to bind to SEMA7A and reduce or eliminate SEMA7A activities and treat a condition).

In certain embodiments and further to paragraphs [0049]-[0055] above, any of the isolated antibodies, full-length or fragments thereof can be part of a pharmaceutical composition that further includes at least one pharmaceutically acceptable excipient. In accordance with these embodiments, pharmaceutical compositions disclosed herein for use to treat, ameliorate a symptom or reduce onset of a health condition in a subject disclosed herein (e.g., to bind to SEMA7A and reduce or eliminate SEMA7A activities).

In some embodiments and further to paragraphs [0049]-[0056] above, an isolated antibody disclosed herein can include, but is not limited to, a heavy chain having a variable region and a constant region. Suitable heavy chain variable regions are described above. In some embodiments, the heavy chain constant region can be derived from an IgG antibody (e.g., a mouse or human IgG antibody). In certain embodiments, the constant region can be a humanized heavy chain constant region. In other embodiments, an isolated antibody disclosed herein can include a heavy chain having a variable region and a constant region. where the heavy chain is represented by the polypeptide sequence represented by SEQ ID NO: 28.

In some embodiments and further to paragraphs [0049]-[0057] above, an isolated antibody disclosed herein can include a light chain having a variable region and a constant region. Suitable light chain variable regions are described herein. In some embodiments, the light chain constant region can be derived from an IgG antibody (e.g., a mouse or human IgG antibody). In other embodiments, the light chain constant region can be derived from a constant region of a kappa or lambda IgG light chain. In other embodiments, the constant region can include a humanized light chain constant region. In other embodiments, an isolated antibody disclosed herein can include a light chain having a variable region and a constant region, where the light chain is represented by the polypeptide sequence represented by SEQ ID NO: 29.

For reference, SEQ ID NOs: 5-29 are provided in Tables 1 and 2 below. Each of the complementary determining regions (CDRs) described herein (e.g., SEQ ID NOs: 9-11 or 13-15) are underlined in the full heavy chain variable regions (SEQ ID NO: 5, 22), light chain variable regions (7 and 23), or full heavy or light chains (SEQ ID NOs: 28 and 29) provided below.

TABLE 1
Amino Acid Sequences of Illustrative Anti-SEMA7 Antibodies

| Description | Sequence | SEQ ID NO: |
|---|---|---|
| Heavy chain | EVQLQQSGTVLARPGASVKMSCKASGYSFTSYWM | 22 |
| variable region (full) | HWVKQRPGQGLEWIGAIYPGNSDTSYNQKFKGKAK | |
| (A) (CDRs | LTAVTSASTAYMELSSLTNEDSAVYYCTRGGAPTMI | |
| underlined) | TAYYAMDYWGQGTSVTVSS | |
| Heavy chain | QVKLQESGTVLARPGASVKMSCKASVYSFTSYWM | 5 |
| variable region (full) | HWVKQRPGQGLEWIGAIYPGNSDTSYNQKFKGKAK | |
| (B) | LTAVTSASTAYMELSSLTNEDSAVYYCTRGGAPTMI | |
| | TAYYAMDYWGQGTSVTVSS | |
| Heavy chain | EVQLQQSGTVLARPGASVKMSCKASGYSFTSYW | 28 |
| (variable + constant − | MHWVKQRPGQGLEWIGAIYPGNSDTSYNQKFKG | |
| -recombinant, with | KAKLTAVTSASTAYMELSSLTNEDSAVYYCTRGG | |
| CDR regions | APTMITAYYAMDYWGQGTSVTVSSAKTTPPSVYP | |
| underlined) | LAPGSAAQTNSMVTLGCLVKGYFPEPVTVTWNSGS | |
| | LSSGVHTFPAVLQSDLYTLSSSVTVPSSTWPSETVTC | |
| | NVAHPASSTKVDKKIVPRDCGCKPCICTVPEVSSVFI | |
| | FPPKPKDVLTITLTPKVTCVVVDISKDDPEVQFSWFV | |
| | DDVEVHTAQTQPREEQFNSTFRSVSELPIMHQDWLN | |
| | GKEFKCRVNSAAFPAPIEKTISKTKGRPKAPQVYTIP | |
| | PPKEQMAKDKVSLTCMITDFFPEDITVEWQWNGQP | |
| | AENYKNTQPIMDTDGSYFVYSKLNVQKSNWEAGNT | |
| | FTCSVLHEGLHNHHTEKSLSHSPGK | |
| Heavy chain | SYWMH | 9 |
| variable region | | |
| CDR-H1 | | |
| Heavy chain | AIYPGNSDTSYNQKFKG | 10 |
| variable region | | |
| CDR-H2 | | |
| Heavy chain | GGAPTMITAYYAMDY | 11 |
| variable region | | |
| CDR-H3 | | |
| Artificial Sequence | RGGAPTMITAYY | 24 |
| of Clone 1271 H1 | | |
| Heavy Chain | | |
| Light chain variable | DIQMTQTTSSLSASLGDRVTISCRASQDISNYLNWY | 23 |
| region (A) | QQKPDGTVKLLIYYTSRLHSGVPSRFSGSGSGTDYSL | |
| (CDRs are | TISNLEQEDIATYFCQQGNTLPWTFGGGTKLEIK | |
| underlined) | | |
| Light chain variable | DIQMTQTTSSLSASLGDRVTISCRASQDISNYLNWY | 7 |
| region (B) | QQKPDGTVKLLIYYTSRLHSGVPSRFSGSGSGTDYSL | |
| (CDRs are | TISNLEQEDIATYFCQQGNTLPWTFGGGTKLEIKR | |
| underlined) | | |
| Full Light Chain | DIQMTQTTSSLSASLGDRVTISCRASQDISNYLNW | 29 |
| (variable + | YQQKPDGTVKLLIYYTSRLHSGVPSRFSGSGSGT | |
| constant) | DYSLTISNLEQEDIATYFCQQGNTLPWTFGGGTK | |
| (CDRs are | LEIKRADAAPTVSIFPPSSEQLTSGGASVVCFLNNFY | |
| underlined | PKDINVKWKIDGSERQNGVLNSWTDQDSKDSTYSM | |
| | SSTLTLTKDEYERHNSYTCEATHKTSTSPIVKSFNRN | |
| | EC | |
| Light chain variable | RASQDISNYLN | 13 |
| region CDR-L1 | | |
| Light chain variable | YTSRLHS | 14 |
| region CDR-L2 | | |
| Light chain variable | QQGNTLPWT | 15 |
| region CDR-L3 | | |

TABLE 2
Nucleic Acid Sequences Encoding Illustrative Anti-SEMA7 Antibodies

| Description | Sequence | SEQ ID NO: |
|---|---|---|
| Nucleic acid | CAAGTCAAGCTGCAGGAGTCTGGGACTGTGCTGGC | 6 |
| encoding Clone | AAGGCCTGGGGCTTCCGTGAAGATGTCCTGCAAGG | |
| 1271H1 heavy | CTTCTGTCTACAGCTTTACCAGCTACTGGATGCACT | |
| chain variable | GGGTAAAACAGAGGCCTGGACAGGGTCTAGAATG | |
| region (full) | GATTGGTGCTATTTATCCTGGAAATAGTGATACTA | |
| | GCTACAACCAGAAGTTCAAGGGCAAGGCCAAACT | |
| | GACTGCAGTCACATCCGCCAGCACTGCCTACATGG | |
| | AGCTCAGCAGCCTGACAAATGAGGACTCTGCGGTC | |
| | TATTACTGTACAAGAGGAGGGGCGCCTACTATGAT | |
| | AACGGCGTACTATGCTATGGACTACTGGGGTCAAG | |
| | GAACCTCAGTCACCGTCTCCTCA | |
| Nucleic acid | GATATCCAGATGACACAGACTACATCCTCCCTGTC | 8 |
| encoding Clone | TGCCTCTCTGGGAGACAGAGTCACCATCAGTTGCA | |
| 1271H1 Light chain | GGGCAAGTCAGGACATTAGCAATTATTTAAACTGG | |
| variable region | TATCAGCAGAAACCAGATGGAACTGTTAAACTCCT | |
| | GATCTACTACACATCAAGATTACACTCAGGAGTCC | |
| | CATCAAGGTTCAGTGGCAGTGGGTCTGGAACAGAT | |
| | TATTCTCTCACCATTAGCAACCTGGAGCAAGAAGA | |
| | TATTGCCACTTACTTTTGCCAACAGGGTAATACGCT | |
| | TCCGTGGACGTTCGGTGGAGGCACCAAGCTGGAAA | |
| | TCAAACGG | |
| Nucleic acid | AGAGGAGGGGCGCCTACTATGATAACGGCGTACTA | 25 |
| encoding Artificial | T | |
| Novel Sequence of | | |
| Clone 1271 H1 | | |
| Heavy Chain | | |
| Alternative nucleic | CGCGGCGGCGCGCCGACCATGATTACCGCGTATTA | 26 |
| acid encoding | T | |
| Artificial Novel | | |
| Sequence of Clone | | |
| 1271 H1 Heavy | | |
| Chain | | |
| Consensus | MGNGGNGGNGCNCCNACNATGATHACNGCNTAYT | 27 |
| sequence encoding | AY | |
| Artificial Novel | where N = any base; M = a or c; H = a or c | |
| Sequence of Clone | or t; and Y = c or t | |
| 1271 H1 Heavy | | |
| Chain | | |

In other embodiments and further to paragraphs [0049]-[0059] above, monoclonal antibodies can be raised against the one or more SEMA7A polypeptide fragments contemplated herein. In some embodiments, a SEMA7A polypeptide fragment is at least 95% identical to amino acids 381-392 of the polypeptide represented by SEQ ID NO:1 or isomer thereof or other SEMA7A molecule thereof. In certain embodiments, the polypeptide fragment construct for use in generating antibodies disclosed herein does not include consecutive amino acids 371-380 or fragment thereof, consecutive amino acids 392-411 or fragment thereof, or both of the polypeptides represented by SEQ ID NO: 1. It is contemplated that truncated or mutated versions of these polypeptides can form part of a construct at least 95% identical to amino acids 381-392 of the polypeptide represented by SEQ ID NO: 1 or isomer thereof. In certain embodiments, the peptide or epitope of SEMA7A of use to generate a monoclonal antibody against SEMA7A is 100% identical to SEQ ID NO: 2.

In some embodiments and further to paragraphs [0049]-[0060] above, an isolated antibody disclosed herein can be a full-length antibody or an antigen-binding fragment thereof. In some embodiments, an isolated antibody disclosed herein can be a full-length antibody, such as an IgG molecule. In other embodiments, an isolated antibody disclosed herein can be an antibody fragment for example, an Fab, a (Fab′) 2, and/or a single-chain antibody. In some embodiments, an isolated antibody disclosed herein can be a human antibody or a humanized antibody. In accordance with these embodiments, any of the referenced isolated antibodies, full-length or fragments thereof can be part of a pharmaceutical composition of use to treat a subject disclosed herein (e.g., to bind to SEMA7A and treat, diagnose, or reduce onset of a health condition).

In some embodiments and further to paragraphs [0049]-[0061] above, an isolated antibody disclosed herein can include a polynucleotide encoding a heavy chain variable region and a polynucleotide encoding a light chain variable region located on a single or two separate vectors. In accordance with these embodiments, the vectors can include one or two of the same or different expression vectors. In certain embodiments, polypeptides disclosed herein are contemplated of use to target SEMA7A and reduce or inhibit SEMA7A activities (e.g., pro-tumor activities). In some embodiments, polypeptides having at least 80%, at least 85%, at least 90% up to 100% polypeptide sequence identity to the polypeptide sequence represented by SEQ ID NO. 24. RGGAPTMITAYY is contemplated. In certain embodiments, the polypeptide can be part of a larger peptide such as an immunoglobulin for example, the polypeptide can be part of an antibody or fragment thereof having anti-SEMA7A activity. In some embodiments, the polypeptide can be about 5 amino acids to about 12 amino acids in length having 100% identity to a contiguous fragment of SEQ ID NO. 24. RGGAPTMITAYY. In other embodiments, the polypeptide can be about 5 amino acids to about 1.000 amino acids in length, or about 10 to about 750 amino acids in length, or about 10 to about 650 amino acids in length, or about 10 to about 600 amino acids in length or about 10 to about 550) amino acids in length or about 50 to about 500 amino acids in length having about 80%, or about 85%, or about 90% or about 95% up to 100% or about 80% up to 100%, sequence identity to a contiguous fragment of SEQ ID NO. 24. RGGAPTMITAYY. In certain embodiments, the polypeptide includes a fragment having at least 90% sequence identity to the polypeptide sequence represented by SEQ ID NO. 24. RGGAPTMITAYY. In other embodiments, the polypeptide includes a fragment having 100% identity within a larger polypeptide than the polypeptide fragment (e.g., fusion peptide, antibody or peptide conjugate or chimera) directed to bind to SEMA7A. In accordance with these embodiments, the polypeptide having about 80% to about 100% amino acid sequence identity to the polypeptide sequence represented by SEQ ID NO. 24. RGGAPTMITAYY as a fragment or part of a larger polypeptide blocks, essentially blocks or completely blocks SEMA7A activity. In certain embodiments, these polypeptide constructs or antibody or monoclonal antibody having about 80% to about 100% polypetide sequence identity to the polypeptide sequence represented by SEQ ID NO. 24. RGGAPTMITAYY inhibits, blocks, or essentially blocks SEMA7A pro-tumor activity. In some embodiments, polypeptides disclosed herein can form part of a chemotherapeutic or anti-tumor agent complex. In certain embodiments. these polypeptides can form part of a pharmaceutical composition of use to treat a condition having aberrant SEMA7A expression and/or activity.

In certain embodiments and further to paragraphs [0049]-[0062] above, the present disclosure includes host cells for generating antibodies contemplated herein. In some embodiments, the host cell can harbor at least one polynucleotide encoding at least one of the heavy chain variable regions disclosed herein and/or a polynucleotide encoding at least one of the light chain variable regions disclosed herein. In some embodiments, a heavy chain variable region can be encoded by a polynucleotide encoding the polypeptide of one or more polypeptide sequences represented by one or more of SEQ ID NOs. 5, 9, 10, 11, 22 and 24. In certain embodiments, a light chain variable region can be encoded by a polynucleotide encoding the polypeptide of one or more polypeptide sequences represented by one or more of SEQ ID NOs. 7, 13, 14, 15, and 23.

In certain embodiments and further to paragraphs [0049]-[0063] above. compositions disclosed herein include pharmaceutical compositions including, but not limited to, one or more monoclonal antibody or fragment thereof and at least one pharmaceutically acceptable excipient. In some embodiments, a pharmaceutical composition disclosed herein can include a full-length antibody and at least one pharmaceutically acceptable excipient. In other embodiments, a pharmaceutical composition disclosed herein can include at least one antibody fragment and at least one pharmaceutically acceptable excipient. In some embodiments, a pharmaceutical composition disclosed herein can include a full-length H1 monoclonal antibody and at least one pharmaceutically acceptable excipient. In some embodiments, a pharmaceutical composition disclosed herein can include a heavy chain variable region represented by a polypeptide sequence represented by any one of SEQ ID NOs. 5, 9, 10, 11, 22 and 24 and at least one pharmaceutically acceptable excipient. In some embodiments, a pharmaceutical composition disclosed herein can include at least one heavy chain variable region represented by a polypeptide sequence represented one or more of SEQ ID NOs. 10, 11, or 24 or biologically relevant fragment therein, and at least one pharmaceutically acceptable excipient. In some embodiments, a pharmaceutical composition disclosed herein can include at least one heavy chain represented by a polypeptide sequence represented at least one of SEQ ID NO. 28 or biologically active fragment thereof, and at least one pharmaceutically acceptable excipient. In addition, these pharmaceutical compositions can include a cell survival inhibitor or checkpoint inhibitor or these agents can be administered separately (e.g., . BCL-2 inhibitor or the like).

In some embodiments and further to paragraphs [0049]-[0064] above, the present disclosure provides isolated antibodies, that bind to SEMA7A with high specificity and reduce or eliminate SEMA7A activities. In certain embodiments. SEMA7A activities include tumor promoting activities such as expansion, metastasis, and migration of tumor cells and/or proinflammatory activities. In certain embodiments, the tumor cells can include, but are not limited to, breast, pancreatic, brain, liver, lung. kidney, or leukemia. In some embodiments, monoclonal antibodies disclosed herein can include monoclonal antibodies directed to bind SEQ ID NO:2. SEQ ID NO:3 or conjugate or fragment thereof (e.g., clone H1 or H1).

In some embodiments and further to paragraphs [0049]-[0065] above, methods disclosed herein include, but are not limited to treating cancer in a subject. In accordance with these embodiments, compositions disclosed herein can be used to reduce or eliminate SEMA7A-related tumor cell metastasis, migration, expansion, or tumor volume or pro-inflammatory activities by blocking or inhibiting these SEMA7A activities using SEMA7A antibody or antibody fragment formulations disclosed herein. In certain embodiments, with respect to treating or ameliorating cancer in a subject, compositions disclosed herein can reduce or eliminate the number of cancer stem cells compared to other SEMA7A antibodies or treatments of cancers expressing SEMA7A without treating the subject with a SEMA7A antibody or fragment thereof. In other embodiments, compositions disclosed herein can be used to diagnose breast cancer, a subclass of breast cancer, breast cancer relapse. survivability, and/or response to standard anti-cancer treatments by obtaining one or more samples from a subject and exposing the one or more samples to a SEMA7A antibody or antibody fragment formulation disclosed herein (e.g., . H1 antibody or fragment thereof-containing composition) and assessing presence, absence or concentration of SEMA7A in the subject.

In some embodiments and further to paragraphs [0049]-[0066] above, combination therapies are contemplated of use to enhance effects of the antibody or fragment thereof and antibody or fragment thereof compositions to reduce onset, prevent, treat or ameliorate SEMA7A-related health conditions including, but not limited to, inflammation (e.g., arthritis such as Rheumatoid arthritis), colitis. IBS, airway inflammation. Gaucher disease), cardiac disease (e.g., such as myocardial infarctions), fibrosis (e.g., liver and/or lung fibrosis), or cancer (e.g., such as breast cancer, leukemia, pancreatic cancer, or brain cancer). In certain embodiments, combination therapies can be used to treat a health condition disclosed herein. In accordance with these embodiments, combination therapies can include, but is not limited to, standard therapies for cancer such as surgical removal of tumors, radiation, chemotherapy. and the like, in combination with or at predetermined intervals with antibody therapies disclosed herein. In other embodiments, combination therapies can include, inhibiting cell survival pathways (e.g., BCL-2) or for example, estrogen receptor interactions (e.g., . PD-L1 or PD-1 inhibition), or immune enhancing therapies in combination with or at predetermined intervals with monoclonal antibody therapies disclosed herein. In other embodiments, other anti-inflammatory treatments can be used before, at the same time, or after the antibody therapies disclosed herein. In yet other embodiments, other cardiac disease treatments can be used before, at the same time or after antibody therapies disclosed herein to treat a subject. In yet other embodiments, other anti-fibrotic medications can be used before, at the same time or after antibody therapies disclosed herein to treat a subject.

In certain embodiments and further to paragraphs [00) 49]-[0067] above, antibodies (e.g., monoclonal antibodies) disclosed herein directed to bind SEMA7A can be used to reduce onset, prevent or treat health conditions associated with SEMA7A. In other embodiments, antibodies (e.g., monoclonal antibodies) disclosed herein directed to bind SEMA7A can be used to diagnose a health conditions associated with SEMA7A. In some embodiments, concentrations of SEMA7A in a sample from a subject can be assessed. In other embodiments, changes in concentrations of SEMA7A in samples from a subject can be assessed for monitoring treatment regimens for further adjustment to a treatment regimen or to combine antibody treatments disclosed herein with known methods to treat the condition (e.g., cancer or inflammation).

In some embodiments and further to paragraphs [0049]-[0068] above, the health condition to be prevented or treated can include, but is not limited to, cancer. In other embodiments, the health condition to be prevented or treated disclosed herein includes, but is not limited to, neurological development, neurological conditions, wound healing, bone homeostasis conditions, angiogenesis issues, pro-inflammatory cytokine release and regulation-related conditions, dendritic cell-related adverse effects on adhesion and motility, fibrosis-related conditions, effects on tumor growth, progression and metastasis (e.g., lymph node metastasis and the like) where inhibition of SEMA7A expression and/or activities reduces the effects of, reduces the onset of, prevents, or treats the condition. In some embodiments, these health conditions including cancers can be treated with SEMA7A monoclonal antibodies generated against SEQ ID NO:2 or SEQ ID NO:3 or conjugate thereof disclosed herein (e.g., H1). In some embodiments, monoclonal antibodies to SEMA7A (e.g., H1) disclosed herein can reduce or eliminate SEMA7A activity such as tumor progression. reduce or inhibit metastasis, shrink tumors, induce tumor cell death, reduce tumor expansion. reduce lymphangiogenesis or angiogenesis lymphovascular invasion and/or reduce lymphogenous/hematogenous metastasis (e.g., reduce infiltration or metastasis of tumor cells into lymph nodes or distant organs) In other embodiments, antibodies against SEMA7A disclosed herein can be used to reduce recruitment of suppressive immune cells such as macrophages and to reduce fibrillar collagen and fibronectin deposition in the tumor microenvironment. In vet other embodiments, SEMA7A presence or levels can be detected in a tumor to assess tumor levels or tumor cell migration. In accordance with these embodiments, if SEMA7A is present or the level has reached a predetermined threshold, the tumor can be treated with one or more SEMA7A monoclonal antibodies to SEQ ID NO.2 or fragment thereof (e.g., H1), to inhibit tumor expansion, shrink tumor size and/or reduce tumor metastasis.

In other embodiments and further to paragraphs [0049]-[0069] above, an isolated SEMA7A antibody or monoclonal antibody disclosed herein binds to an epitope on a polypeptide having a polypeptide sequence represented by SEQ ID NO: 2 or 3 or isomer equivalent thereof or fragment thereof where binding the epitope inhibits SEMA7A expression and/or activities (e.g., H1 clone). In some embodiments, the isolated SEMA7A antibody or monoclonal antibody disclosed herein binds to an epitope on a polypeptide having a polypeptide sequence represented by SEQ ID NO: 2 or 3 or isomer equivalent thereof or fragment thereof and can kill tumor cells or induce apoptosis of tumor cells leading to tumor cell death reducing or eliminating metastasis and/or tumor volume. In certain embodiments, the tumor cells can include breast cancer cells. In some embodiments, the isolated SEMA7A antibody or monoclonal antibody is a full-length antibody or SEMA7A binding fragment thereof, such as a heavy chain region thereof (e.g., SEQ ID NO: 5, 6, 9, 10, 11, 22 or 24). In some embodiments, the isolated anti-SEMA7A antibody is a full-length monoclonal antibody. In certain embodiments, the isolated anti-SEMA7A antibody is a single-chain antibody (scFv).

In other embodiments and further to paragraphs [0049]-[0070] above, a polynucleotide encoding a SEMA7A antibody, monoclonal antibody or antigen binding fragment thereof provided herein is contemplated. In accordance with these embodiments, the polynucleotide can further include a vector for expressing the encoded antibody or antigen binding fragment. In yet other embodiments, host cells harboring the polynucleotide encoding an SEMA7A antibody, monoclonal antibody or antigen binding fragment thereof are included herein.

In some embodiments and further to paragraphs [0049]-[0071] above. pharmaceutical compositions are disclosed containing at least one anti-SEMA7A antibody, monoclonal antibody or polynucleotide expressing the at least one antibody as described herein (e.g., that binds to a sequence that is at least 75%, at least 80%, at least 85%, at least 90%, at least 95%, up to 100% homologous to the sequence represented by SEQ ID NO:2, or fragment thereof) and a pharmaceutically acceptable carrier or excipient of use for treating health conditions are provided. In accordance with these embodiments, the pharmaceutical compositions can further include an anti-inflammatory agent, an anti-cancer agent, a non-specific innate immune response stimulator, or other standard agent of use in combination with the antibodies and monoclonal antibodies disclosed herein, for example to treat conditions expressing or over-expressing SEMA7A. It is noted that the antibodies, monoclonal antibodies, and fragments thereof bind to any SEMA7A including any isomeric form of SEMA7A or mutant form thereof. In some embodiments, the pharmaceutical compositions can further include one or more of an anti-microbial agent, a chemotherapeutic agent, and/or immunostimulatory or anti-inflammatory agent depending on the health condition to be treated. Combination therapies that include antibodies disclosed herein are contemplated as well as combining antibody treatments with standard surgical procedures for treating the health condition. In yet other embodiments, the composition or pharmaceutical compositions disclosed herein can include a monoclonal antibody clone designated as H1 or fragment thereof, or a combination thereof of use in single or multiple dosing regimens to treat a subject in need thereof of such a treatment.

In some embodiments and further to paragraphs [0049]-[0072] above, methods include administering a composition disclosed herein and reduce or eliminate SEMA7A activities (e.g., induce anti-tumor activities) in the subject in need thereof. In accordance with these embodiments, pharmaceutical compositions described herein can be administered to the subject by any means known in the art for administering an antibody, monoclonal antibody. or fragment thereof. In another embodiment, methods are provided for diagnosing and/or treating cancer in the subject.

In some embodiments and further to paragraphs [0049]-[0073] above, cancers to be treated by compositions containing monoclonal antibodies disclosed herein include, but are not limited to, a solid tumor. In certain embodiments, the solid tumor includes, but is not limited to, breast, prostate, liver, lung, kidney, stomach, ovarian, intestinal, head, neck, brain, skin, testicular, ovarian, pancreatic or another solid tumor. In certain embodiments, when treating solid tumors, the pharmaceutical compositions can be administered systemically. topically, by bolus infusion, intranasally, intravenously, subcutaneously, by slow-release, by seed implant, orally, or by direct local administration into the solid tumor of the subject.

In some embodiments and further to paragraphs [0049]-[0074] above, compositions, and methods for inhibiting SEMA7A expression and/or activity disclosed herein can be used to reduce or prevent expansion, migration, metastasis and/or maturation of cancer stem cells. In certain embodiments, the cancer stem cells can include any cancer stem cell of any origin. In other embodiments, the cancer stem cells include any cancer stem cell resistant to radiation, chemotherapy, or other anti-cancer treatment where compositions and methods disclosed herein can be used to reduce or prevent cancer stem cell activities and survival to treat a subject (e.g., completely treat a subject to a cancer free stage) in need thereof. In other embodiments, the cancer stem cells include any cancer stem cell sensitive to anti-SEMA7A antibody treatment where compositions and methods disclosed herein can be used to reduce or prevent cancer stem cell activities and survival to treat a subject in need thereof. In other embodiments, cancer stem cells disclosed herein can include mammary cells and/or cells of mammary tissues (e.g., epithelial cells). In another embodiment, compositions and methods disclosed herein can be used to inhibit metastasis of cancer stem cells, treat a tumor and/or reduce anoikis resistance in a subject. In certain embodiments, the subject is a female subject having post-partum breast cancer (PPBC). In certain embodiments, antibodies against SEMA7A can be used to reduce or prevent expansion, migration, metastasis and/or maturation of cancer stem cells. In some embodiments, compositions and methods disclosed herein can be used to reduce expression and/or activity of SEMA7A in normal cells to treat a subject.

In certain embodiments and further to paragraphs [0049]-[0075] above, kits are contemplated for use in storing, transporting, and using monoclonal antibodies or fragments thereof (e.g., isolated monoclonal antibodies, pharmaceutical compositions disclosed herein. host cells) disclosed herein. In some embodiments, kits are contemplated for practicing any of the methods disclosed herein (e.g., treating/inhibiting a subject having a condition expressing or overexpressing SEMA7A, generating isolated antibodies, generating pharmaceutical compositions, and/or generating host cells) disclosed herein.

Other embodiments and further to paragraphs [0049]-[0076] above, relate to methods of making and methods of using antibodies for prevention, treatment and/or diagnosis of a condition such as using monoclonal antibodies against a pre-selected peptide or active fragments thereof, represented by SEQ ID NO: 2 or SEQ ID NO:3, or biologically active fragment thereof of SEMA7A. In some embodiments, these monoclonal antibodies or fragments thereon disclosed herein are capable of binding to SEMA7A and treating a condition expressing or over-expressing SEMA7A. In certain embodiments, a monoclonal can be conjugated to a carrier molecule (e.g., . BSA or KLH) or other molecule or a fusion polypeptide of the polypeptide represented by SEQ ID NO: 2 or SEQ ID NO:3 or simply the polypeptide represented by SEQ ID NO: 2 or SEQ ID NO:3 of use to generate and use monoclonal antibodies to diagnose, ameliorate and/or treat a subject having a health condition associated with expression of, or over-expression of SEMA7A. In accordance with these embodiments. SEMA7A monoclonal antibodies were generated against SEQ ID NO:2 or 3. In some embodiments, these monoclonal antibodies can be used to target and kill tumor cells including, non-stem cell cancer cells and cancer stem cells in a subject in need thereof. In yet other embodiments, monoclonal antibodies, or biologically active fragment thereof against SEQ ID NO:2 or 3 can be used in combination with one or more cell survival pathway inhibitor (e.g., . BCL-2 inhibitor. P13K pathway inhibitor, or the like) and/or one or more of a beta-1 integrin or Akt inhibitors. In some embodiments, combination therapies can include monoclonal antibodies, or biologically active fragment thereof against SEQ ID NO:2 or 3 and one or more PI3K (e.g., . LY294002) inhibitor and/or Src inhibitor (e.g., . SU6656) in order to reduce tumor cell viability or eliminate tumor cells and/or cancer stem cells expressing SEMA7A or other marker. In certain embodiments, these treatments can be timed to treat post-partum breast cancer patients at risk, or breast cancer patients at high risk of breast cancer relapse. In accordance with these embodiments, combinations disclosed herein can target and kill tumor cells by blocking the mechanisms of action by which SEMA7A promotes tumor cell growth and survival.

In some embodiments and further to paragraphs [0049]-[0077] above, monoclonal antibody clones contemplated herein can include clones referenced as H1 or H7 or other monoclonal antibody thereof. Other monoclonal antibody clones are contemplated of use in compositions and methods disclosed herein generated to the peptides of some embodiments provided herein. In certain embodiments. SEMA7A monoclonal antibody clones generated against SEQ ID NO:2 or SEQ ID NO:3 or biologically active fragment thereof and/or referenced herein as monoclonal antibody clone H1 can be used to treat cancer. In other embodiments, the cancer is a solid tumor expressing SEMA7A. In certain embodiments, the cancer is a non-solid cancer of the blood (e.g., leukemia). In yet other embodiments, the cancer is breast cancer. In certain embodiments, the breast cancer is ER+BC. ER-BC, or postpartum breast cancer (PPBC) or triple negative breast cancer. In some embodiments, the PPBC breast cancer is diagnosed within 1 year, to within 2, to within 3, to within 5, to within or after 10 years of the latest childbirth of a subject. In certain embodiments, monoclonal antibodies generated against SEQ ID NO:2 are used to diagnose cancer and diagnose cancer progression, relapse and/or cancer survival. In some embodiments, the pharmaceutical composition disclosed herein can be used to treat breast cancer and reduce or prevent lymphovascular invasion (LVI) and lymph node(s) (LN) involvement. In accordance with these embodiments, the subject can be treated daily, weekly, monthly, or other regimen or at a time when SEMA7A levels are detectable, are used to diagnose cancer and diagnose cancer progression, relapse and/or cancer survival. In some embodiments, the cancer is breast cancer.

### SEMA7A

SEMATA is a member of the Semaphorin family of axon guidance molecules and, when expressed on human erythrocytes, is the John Milton Hagen (JMH) blood group antigen. SEMA7A is an ˜80 kDa membrane-anchored glycoprotein that contains an RGD integrin interaction motif within its full-length amino acid sequence.

### Cancers

Cancer is one of the leading health issues globally and one of the leading causes of death in humans and other mammals. Breast cancer, for example, is a global health threat with an estimated 1.7 million cases annually. Death due to breast cancer disproportionately affects low-income countries and younger women in these countries are particularly at risk. In the U.S., about 27,000 younger women are affected by breast cancer annually, with age of less than 35 years old and diagnosis within ten years of most recent childbirth as two unique risk factors for breast cancer metastasis and death. Pregnancy-associated breast cancer (PABC) has been defined as any breast cancer diagnosed during pregnancy or in the 1-2 years immediately after childbirth. However, significant research has clarified that women diagnosed with breast cancer during pregnancy fare equal to their non-pregnant peers with similar breast cancers. However, women diagnosed in the early postpartum years face significant increased risk for metastasis in comparison to nulliparous women or women whose children are older, despite their breast cancers having similar risk profiles. Therefore, in certain instances it is important to separate PABC, especially those diagnosed during pregnancy, from postpartum breast cancer (PPBC) as the outcomes can vary in these groups.

PPBC is currently defined as a breast cancer diagnosis occurring within ten years following childbirth. In one study, patients diagnosed with PPBC experienced a 2-fold increase in mortality risk when diagnosed 4-6 years following childbirth, while patients within two years postpartum experienced a thirty percent increase in mortality rate. In other studies, where detailed tumor characteristics were available, patients diagnosed within 5 years of giving birth had about a three-fold risk of metastatic recurrence without a significant difference present in the clinical risk characteristics of the tumors, as compared to nulliparous or patients whose most recent childbirth occurred more than ten years before diagnosis. This increased risk period was then extended when a larger cohort study demonstrated that postpartum patients diagnosed with Stage 1 and 2 tumors up to ten years from their most recent childbirth were at a 3 to 5-fold significantly higher risk for developing metastases. with tumors continuing to recur up to 15 years after diagnosis. In these studies, a postpartum diagnosis was associated with a higher incidence of having recurrence and poor treatment response as well as metastasis. In certain embodiments, treatments disclosed herein can reduce or eliminate metastases, reduce, or eliminate lymphovascular invasion (LVI) and/or reduce or eliminate lymph nodes (LN) lymphovascular invasion (LVI) and involved lymph node(s) (LN), involvement, or a combination thereof in cancers expressing or over-expressing SEMA7A.

In certain embodiments and further to paragraphs [0049]-[0081] above, antibodies disclosed herein that target SEMA7A and SEMA7A activities can be used to reduce pro-tumor activities, enhance anti-tumor responses to shrink or eliminate cancerous growth and/or reduce or prevent tumor expansion and/or kill tumor cells. In certain embodiments, the anti-SEMA7A antibodies disclosed herein can be used in conjunction with another appropriate active agent (e.g., a chemotherapeutic agent or other targeted anti-cancer therapy, an antibiotic, an anti-fungal, an anti-viral, or a non-specific immune stimulator).

In certain embodiments and further to paragraphs [0049]-[0082] above. compositions disclosed herein can be used to diagnose the presence of SEMATA in a subject having cancer which can correlate with unresponsiveness to standard therapies and relapse in certain subjects having cancer. In accordance with these embodiments, diagnosing presence of SEMATA in a subject having cancer can reduce or eliminate needless treatment with more toxic agents or treatment such as radiation or chemotherapy depending on the tumor type to avoid adverse effects due to these agents. In certain embodiments, detection of SEMA7A in a subject having breast cancer and/or PPBC can alert the caretaker and eliminate treatment regimens known to not be unsuccessful in treating these tumors, avoiding anti-cancer toxicities to focus on treatments, for example, cell survival pathway inhibitors and/or blocking SEMA7A using an antibody treatment disclosed herein to treat the subject.

In some embodiments and further to paragraphs [0049]-[0083] above, antibody or monoclonal antibody therapies disclosed herein can be used before, after or simultaneously with other treatments such as radiation, chemotherapy, anti-inflammatory or other targeted anti-cancer therapies or immunotherapies. In some embodiments, antibodies to SEMA7A disclosed herein can be administered in combination with a non-specific immune stimulator composition, containing, for example, a TLR ligand. T-cell stimulant, or other immunostimulatory such as CTLA-4 or the like or anti-cancer agent during, at the time of, or after treating a tumor with one or more antibodies disclosed herein. In accordance with these embodiments, these combinations can act synergistically to enhance anti-tumor or anti-SEMA7A activities to treat cancer or other health condition contemplated herein. In certain embodiments, the present disclosure includes host cells of use in generating antibodies contemplated herein. In other embodiments, a host cell can include a polynucleotide encoding at least one of the heavy chain variable regions disclosed herein and/or a polynucleotide encoding at least one of the light chain variable regions disclosed herein. In certain embodiments, a host cell can include a polynucleotide encoding the polypeptide of one or more polypeptide sequence represented by SEQ ID NOs. 5, 9, 10, 11, 22 and 24. In certain embodiments, a host cell can include a polynucleotide encoding the polypeptide of one or more polypeptide sequence represented by SEQ ID NOs. 7, 13, 14, 15, and 23. In certain embodiments, the host cell can include a polynucleotide having a nucleotide sequence represented by SEQ ID NOs: 6, 8. 25, 26 or 27 (Table 2, above). In certain embodiments, a monoclonal antibody of use herein can include a heavy chain variable region of one or more polypeptide sequence represented by SEQ ID NOs. 5, 9, 10, 11, 22, and 24 and a light chain variable region of one or more polypeptide sequence represented by SEQ ID NOs. 7, 13, 14, 15, and 23 or any combination thereof.

In certain embodiments and further to paragraphs [0049]-[0084] above, the present disclosure provides pharmaceutical compositions containing one or more monoclonal antibody or fragment thereof and a pharmaceutically acceptable excipient. In some embodiments, a pharmaceutical composition disclosed herein includes a full-length antibody and a pharmaceutically acceptable excipient. In other embodiments, a pharmaceutical composition disclosed herein includes at least one antibody fragment and a pharmaceutically acceptable excipient. In some embodiments, a pharmaceutical composition disclosed herein includes a full-length H1 monoclonal antibody and a pharmaceutically acceptable excipient. In some embodiments, a pharmaceutical composition disclosed herein includes a heavy chain variable region represented by an amino acid sequence represented by any one of SEQ ID NOs. 5, 9. 10, 11, and 24 and a pharmaceutically acceptable excipient. In other embodiments, a pharmaceutical composition disclosed herein can include a heavy chain variable region represented by an amino acid sequence represented by SEQ ID NOs. 10. orl land a pharmaceutically acceptable excipient. In addition, these compositions can further include or be administered separately from, a cell survival inhibitor (e.g., . BCL-2 inhibitor or the like)

In yet other embodiments and further to paragraphs [0049]-[0085] above, cell survival pathway inhibitors (e.g., anti-BCL-2, anti-P13K inhibitors. NF—KB or STAT3 inhibitors), anti-inflammatory treatments, cardiac medications, or anti-fibrotic medication can be used to supplement any monoclonal antibody treatment disclosed herein before, during or after these antibody treatments.

In certain embodiments and further to paragraphs [0049]-[0086] above, a dosing regimen described herein can include administering a SEMA7A antibody or monoclonal antibody disclosed herein to a subject as described herein as a direct injection (e.g., into a tumor such as a solid tumor), systemically, topically, intranasally, subcutaneously, in a bolus or in slow-release such as microparticles or other slow-release form to a subject. In other embodiments, a dosing regimen can include, but is not limited to, administering an antibody systemically (e.g., using parenteral administration). In certain embodiments, a dosing regimen can further include, but is not limited to, administering additional active agents (as described herein). In accordance with these embodiments, certain routes of administration suitable for delivering an SEMA7A antibody composition are described in more detail below.

In other embodiments and further to paragraphs [0049]-[0087] above, one or more peptides disclosed herein can be of use alone as the peptide or further be conjugated, carrier protein, or part of a fusion polypeptide (e.g., having a protein transduction domain such as tat or a fusion polypeptide having an Fc or other molecule, etc.). In accordance with these embodiments, these peptides or conjugates or fusion polypeptides thereof can be part of a composition or pharmaceutical composition contemplated herein. In other embodiments, these compositions can be pharmaceutical compositions and can be introduced to a subject to reduce the risk of onset (e.g., all or part of a tumor vaccine composition) and/or treat a health condition having SEMA7A expression or over expression. In certain embodiments, one or more monoclonal antibodies directed to bind SEMA7A derived from selection against a polypeptide having at least 75% homology, at least 80% homology, at least 85% homology to SEQ ID NO: 2 or SEQ. ID NO: 3 are contemplated to be part of these pharmaceutical compositions for treating a subject having or suspected of developing cancer or other health condition having adverse SEMA7A involvement. Any method for introducing or administering a polypeptide or polypeptide-containing composition to a subject in need thereof is contemplated such as orally, subcutaneously, intravenously, by bolus, by slow- or timed-release formulation, intranasally, direct application, or combination administration thereof.

In other embodiments and further to paragraphs [0049]-[0088] above, compositions, and methods for inhibiting SEMA7A expression and/or activity disclosed herein can be used to reduce or prevent expansion, transformation, migration, stemness, metastasis and/or maturation of cancer stem cells found within a tumor or remaining after standard anti-tumor therapies. In certain embodiments, cancer stem cells include any cancer stem cell of any origin. In other embodiments, the cancer stem cells include any cancer stem cell resistant to radiation, chemotherapy, surgical removal, or other anti-cancer treatment wherein compositions and methods disclosed herein can be used to reduce or prevent cancer stem cell activities and survival to fully treat a subject in need of such a treatment. In other embodiments, cancer stem cells disclosed herein can include, but are not limited to. mammary cells and/or cells of mammary tissues (e.g., epithelial cells. MECs) or other cancer stem cells. In another embodiment, monoclonal antibody or polyclonal antibody compositions and methods disclosed herein can be used to inhibit metastasis of cancer stem cells, treat a tumor and/or reduce anoikis resistance in a subject. In certain embodiments, the subject is a female subject having PPBC. In other embodiments, antibodies against SEMA7A can be used to reduce or prevent expansion, migration, metastasis and/or maturation of cancer stem cells. In some embodiments, compositions and methods disclosed herein can be used to reduce expression, maturation, transformation, stemness, metastasis, and/or activity of SEMA7A in normal cells to treat a subject. In other embodiments, compositions, and methods for inhibiting SEMA7A expression can be used to reduce occurrence and/or expansion of drug resistant tumor cells such as stem cells or other tumor cells.

In certain embodiments and further to paragraphs [0049]-[0089] above. compositions, and methods for inhibiting SEMA7A expression and/or activity disclosed herein can be used before, during or after standard anti-tumor therapies. In accordance with these embodiments, any agent capable of inhibiting SEMA7A expression and/or activity can be used to reduce or eliminate cancer stem cells and/or reduce or eliminate development of drug resistance cancer cells and/or reduce cellular transformation of normal to a cancerous cell type in a subject. In some embodiments, compositions and methods disclosed herein can be used during and/or after standard anti-cancer therapies. In other embodiments. compositions, and methods for inhibiting SEMA7A expression and/or activity can be used following radiation therapy, chemotherapy, surgery, or other tumor reducing treatment where agents capable of inhibiting SEMA7A (e.g., antibodies, chemical agents, snRNAs etc.) can be used to treat the remaining tumor cells including cancer stem cells to reduce or eliminate tumor cells including cancer stem cells or metastasis of these cells. In certain embodiments, the subject has breast cancer. In some embodiments, the breast cancer is PPBC. In accordance with these embodiments, a subject having PPBC has had or is undergoing postpartum mammary gland involution and can be treated as disclosed herein targeting cancer stem cells before, during or after standard treatments of care.

Isolated anti-SEMA7A Antibodies

In some embodiments and further to paragraphs [0049]-[0090] above, an isolated or synthetic SEMA7A antibody or monoclonal antibody or fragment thereof is provided. As used herein, the terms “SEMA7A antibody” or “antibody” can refer to an antibody capable of binding to and blocking expression of or activities of SEMA7A. In certain embodiments. SEMA7A polypeptides can include an amino acid sequence having 75% or 85% or more (e.g., 85%, 86%, 87%, 88%, 89%, 90%, 91%, 92%, 93%, 94%, 95%, 96%, 97%, 98%, 99%, or 100%) sequence identity to SEQ ID NO: 2 or 3 or isomeric equivalent thereof or amino acids 381-392 of SEQ ID NO: 1 are contemplated of use to diagnose cancer and intervene as needed to treat cancer in the subject. In some embodiments, SEMA7A polypeptides of use herein can be conjugated to a carrier protein such as BSA or KLH or other carrier and can further include a linker (e.g., 20 amino acid or less. 3-mer. 4-mer) that separates the carrier from the peptide. In certain embodiments. SEMA7A polypeptides can include a polypeptide sequence of at least 80%, or at least 85% or more, up to 100% identical in sequence identity to SEQ ID NO: 2 or 3 or amino acids 381-392 of SEQ ID NO: 1 conjugated to a carrier with an amino acid linker between for use in generating one or more monoclonal antibodies. These monoclonal antibodies can be used in pharmaceutical compositions to diagnose, prognose. ameliorate, prevent, or treat SEMA7A expressing or overexpressing health conditions in a subject (e.g., cancer, such as breast cancer). In certain embodiments, these monoclonal antibodies can be used to kill cancer cells including cancer stem cells in a subject to ameliorate, prevent, or treat SEMA7A expressing or overexpressing health conditions in the subject.

In other embodiments and further to paragraphs [0049]-[0091] above, isolated or synthetic SEMA7A antibodies or monoclonal antibodies or fragments thereof described herein can specifically bind to an epitope on a polypeptide having an amino acid sequence represented by amino acids 381-392 of SEQ ID NO: 1 or isomer thereof. In other embodiments amino acids 381-392 of SEMA7A can be included as part of a longer polypeptide such as at least a 12mer. 14mer. 16mer. 18mer. 20mer. 22mer. 24mer, 26mer. 28mer. 30mer. 32mer. 34mer. 36mer. 38mer or 40mer or longer or other appropriate length polypeptide or a fragment thereof such as a 6mer or 8mer of this selected region. In certain embodiments, the peptide can be shorter than 40 amino acids in length. In other embodiments, the peptide can form part of a fusion polypeptide. In accordance with these embodiments, one or more of these polypeptides can be used to raise polyclonal and/or monoclonal antibodies of use herein.

In other embodiments and further to paragraphs [0049]-[0092] above, isolated or synthetic SEMA7A antibodies or monoclonal antibodies or fragments thereof described herein can specifically bind to an epitope on a polypeptide having an amino acid sequence represented by amino acids 381-392 of SEQ ID NO: 1 or isomer thereof without binding to amino acids 371-380 and/or 393-441 of SEQ ID NO: 1 or isomer thereof. In other embodiments, isolated or synthetic SEMA7A antibodies or monoclonal antibodies or fragments thereof described herein can specifically bind to an epitope on a polypeptide having an amino acid sequence represented by amino acids 381-392 of SEQ ID NO: 1 or isomer thereof without binding to more than five amino acids of amino acids 371-380 and/or five amino acids of amino acids 393-441 of SEQ ID NO: 1. In certain embodiments. SEMA7A antibodies or monoclonal antibodies specifically bind to the polypeptide having an amino acid sequence represented by amino acids 381-392 of SEQ ID NO: 1 (ADRHPEVAQR SEQ ID NO:2: conjugated polypeptide with 3-mer linker: CGGADRHPEVAQR SEQ ID NO:3) with high affinity. In some embodiments, antibodies binding this epitope have superior anti-SEMA7A activity (e.g., tumor cell killing, improved anti-tumor activities) compared to commercially available antibodies against amino acids 1-100 of SEQ ID NO: 1 or amino acids 371-441 of SEQ ID NO: 1, for example.

In other embodiments and further to paragraphs [0049]-[0093] above, isolated or synthetic SEMA7A antibodies or monoclonal antibodies or fragments thereof described herein can specifically bind to an epitope unique to the human SEMA7A protein wherein any human isomer is contemplated. In other embodiments, isolated or synthetic SEMA7A antibodies or monoclonal antibodies or fragments thereof described herein cannot bind to a non-human SEMA7A protein.

In some embodiments, compositions of use herein can include antibodies binding to SEMA7A. An antibody (interchangeably used in plural form) is an immunoglobulin molecule capable of specific binding to a target, such as a carbohydrate, polynucleotide, lipid. polypeptide, etc., through at least one antigen recognition site, located in the variable region of the immunoglobulin molecule. As used herein, the term “antibody” encompasses not only intact (e.g., full-length) polyclonal or monoclonal antibodies, but also antigen-binding fragments thereof (such as Fab, Fab′, F(ab′)₂, Fv), single-chain antibody (scFv), fusion proteins comprising an antibody portion (e.g., chimeric antigen receptor or CAR), humanized antibodies, chimeric antibodies, diabodies, single domain antibody (e.g., a VH only antibody such as a nanobody), multi-specific antibodies (e.g., bispecific antibodies) and any other modified configuration of the immunoglobulin molecule that includes an antigen recognition site of the required specificity, including glycosylation variants of antibodies, amino acid sequence variants of antibodies, and covalently modified antibodies. An antibody encompassed herein can include an antibody of any class, such as IgD. IgE. IgG. IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA. IgD, IgE, IgG, and IgM, and several of these can be further divided into subclasses (isotypes), e.g., . IgG1, IgG2, IgG3, IgG4, IgA1 and IgA2. The heavy-chain constant domains that correspond to the different classes of immunoglobulins are called alpha, delta, epsilon, gamma, and mu, respectively. An antibody molecule encompassed herein can include a heavy chain variable region (VH) and a light chain variable region (VL). The VH and VL regions can be further subdivided into regions of hypervariability, also known as “complementarity determining regions” (“CDR”), interspersed with regions that are more conserved, which are known as “framework regions” (“FR”). Each VH and VL can be composed of three CDRs and four FRs, arranged from amino-terminus to carboxy-terminus in the following order: FRI. CDR1. FR2. CDR2. FR3. CDR3. FR4. The extent of the framework region and CDRs can be precisely identified using methodology known in the art.

In certain embodiments and further to paragraphs [0049]-[0095] above, antibodies described herein can specifically bind to a corresponding target antigen (e.g., . SEMA7A. SEQ ID NO: 2, a representative epitope) or an epitope thereof. An antibody that “specifically binds” to an antigen or an epitope is a term well understood in the art. A molecule is said to exhibit “specific binding” if it reacts more frequently, more rapidly, with greater duration, and/or with greater affinity with a particular target antigen than it does with alternative targets. An antibody “specifically binds” to a target antigen or epitope if it binds with greater affinity, avidity, more readily, and/or with greater duration than it binds to other substances. As an example, an antibody that specifically (or preferentially) binds to an antigen (e.g., . SEMA7A. SEQ ID NO:2, a representative epitope) or an antigenic epitope therein is an antibody that binds this target antigen with greater affinity, avidity, more readily, and/or with greater duration than it binds to other antigens or other epitopes in the same antigen. It is also understood with this definition that, as an example, an antibody that specifically binds to a first target antigen may or may not specifically or preferentially bind to a second target antigen. As such. “specific binding” or “preferential binding” does not necessarily require (although it can include) exclusive binding. In some examples, an antibody that “specifically binds” to a target antigen or an epitope thereof cannot bind to other antigens or other epitopes in the same antigen (i.e., only baseline binding activity can be detected in a conventional method).

A typical antibody molecule includes a heavy chain variable region (VH) and a light chain variable region (VL), which are usually included in antigen binding. The VH and VL regions can be further subdivided into regions of hypervariability, also known as “complementarity determining regions” (“CDR”), interspersed with regions that are more conserved, which are referred to as “framework regions” (“FR”). Each VH and VL is typically composed of three CDRs and four FRs, arranged from amino-terminus to carboxyl-terminus in the following order: FR1, CDR1, FR2, CDR2, FR3, CDR3, FR4. The extent of the framework region and CDRs can be precisely identified using methodology known in the art.

In some embodiments and further to paragraphs [0049]-[0097] above, provided herein are representative SEMA7A antibodies: monoclonal SEMA7A antibody clone H1 and monoclonal SEMA7A antibody clone H7-both for binding SEMA7A. In certain embodiments, antibodies can include a VH region having 85% or more (e.g., 90%, 95%. 98%, 99% or more) sequence identity to the VH region of monoclonal SEMA7A antibody clone H1. In certain embodiments, antibodies can include a VL region having 80% or more. 85% or more (e.g., 90%, 95%, 98%, 99% or more) sequence identity to the VL region of monoclonal SEMA7A antibody clone H1.

In certain embodiments and further to paragraphs [0049]-[0098] above, an isolated SEMA7A antibody can be a full-length antibody, which contains two heavy chains and two light chains, each including a variable domain and a constant domain. In some embodiments, each heavy chain (comprising a variable domain and a constant domain) can include, but is not limited to. SEQ ID NO: 28. In some embodiments, each light chain (comprising a variable domain and a constant domain) can include, but is not limited to. SEQ ID NO: 29. In certain embodiments, an isolated SEMA7 antibody can be an antigen binding fragment of a full-length antibody capable of binding SEMA7A and of use to treat cancer or other health conditions. In accordance with these embodiments, an SEMA7A antibody or antigen binding fragment thereof described herein can have binding fragments encompassed within the term “antigen-binding fragment” of a full length antibody can include (i) a Fab fragment, a monovalent fragment consisting of the VL, VH, CL and CHI domains: (ii) a F(ab′) 2 fragment, a bivalent fragment including two Fab fragments linked by a disulfide bridge at the hinge region: (iii) a Fd fragment consisting of the VH and CHI domains: (iv) a Fv fragment consisting of the VL and VH domains of a single arm of an antibody. (v) a dAb fragment, which consists of a VH domain; and (vi) an isolated complementarity determining region (CDR) that retains functionality. In accordance with these embodiments, a SEMA7A antibody disclosed herein can have two domains of the Fv fragment wherein, although the two domains of the Fv fragment, VL and VH, are coded for by separate genes, they can be joined, using recombinant methods, by a synthetic linker that enables them to be made as a single protein chain in which the VL and VH regions pair to form monovalent molecules known as single chain Fv (scFv).

In some embodiments and further to paragraphs [0049]-[0099] above, isolated SEMA7A antibodies disclosed herein can be a single chain antibody (scFv). In accordance with some embodiments herein, a scFv antibody can be a VH fragment and a VL fragment, which can be linked via a flexible peptide linker. In some embodiments, a scFv antibody herein can be in the VH→VL orientation (from N-terminus to C-terminus). In some embodiments, a scFv antibody can be in the VL→VH orientation (from N-terminus to C-terminus).

### Preparation and Isolation of Antibodies

In certain embodiments and further to paragraphs [0049]-[00100] above, antibodies capable of binding to SEMA7A as described herein can be made by any method known in the art. In some embodiments, monoclonal antibodies herein can be generated using a conventional hy bridoma technology and/or by isolating from an antibody library (e.g., obtained from antisera of an immunized animal). Isolated antibodies can be screened for a specific binding affinity to SEMA7A (and not to other targets). Once antibodies generated with high binding affinity for SEQ ID NO:2 or 3 or isomer thereof or antibodies generated with high binding affinity of conjugated polypeptides thereof are identified, the SEMA7A antibodies can be sequenced and recombinantly expressed, and then administered in an in vivo, in situ, or in vitro setting to analyze the ability of the antibodies to and block SEMA7A and its activities. In certain embodiments, these antibodies bind with high affinity to SEMA7A in purified form, in a sample such as a tumor or blood sample. In some embodiments, these antibodies are prepared as mammalian or humanized antibodies.

### Generating Antibodies

### Hybridoma Technology

In some embodiments, antibodies specific to a target antigen (e.g., . SEMA7A SEQ ID NO: 2 or 3 or isomer thereof) can be made by conventional hybridoma technology. In some embodiments, the full-length target antigen or a fragment thereof, optionally coupled to a carrier protein such as KLH, can be used to immunize a host animal (e.g., a mouse or a canine) for generating antibodies binding to that antigen. In some embodiments, the route and schedule of immunization of the host animal are generally in keeping with established and conventional techniques for antibody stimulation and production, as further described herein. In other embodiments, it is contemplated that any mammalian subject can be manipulated to serve as the basis for production of mammalian, including mouse hybridoma cell lines. In some embodiments, the host animal can be inoculated intraperitoneally. intramuscularly, orally, subcutaneously, intraplantar, and/or intradermally with an amount of immunogen, including as described herein. In some embodiments, immunization of a host animal with a target antigen SEQ ID NO: 2 or 3 or isomer thereof, or a fragment containing the target amino acid sequence conjugated to a protein that is immunogenic in the species to be immunized, e.g., keyhole limpet hemocyanin (KLH), serum albumin (e.g., bovine serum albumin (BSA)), bovine thyroglobulin, or soy bean trypsin inhibitor using a bifunctional or derivatizing agent, for example maleimidobenzoyl sulfosuccinimide ester (conjugation through cysteine residues). N-hydroxysuccinimide (through lysine residues), glutaraldehyde, succinic anhydride, SOCl, or R1N═C═NR, where R and RI are different alkyl groups, can yield a population of antibodies for use herein.

In some embodiments and further to paragraphs [0049]-[00102] above, hybridomas can be prepared from the lymphocytes and immortalized myeloma cells using the general somatic cell hybridization techniques known in the art. Available myeloma lines can be used. In some embodiments, the technique that can be used herein can include fusing myeloma cells and lymphoid cells to produce hybridomas. In some embodiments, cell fusion technique. EBV immortalized B cells can be used to produce the SEMA7A monoclonal antibodies described herein. In accordance with these embodiments, hybridomas can be expanded and subcloned, and supernatants can be assayed for anti-immunogen activity by conventional immunoassay procedures (e.g., radioimmunoassay, enzyme immunoassay, or fluorescence immunoassay). Substitute Specification

In certain embodiments and further to paragraphs [0049]-[00103] above, hybridomas that can be used as source of antibodies herein can encompass all derivatives, progeny cells of the parent hybridomas that produce monoclonal antibodies capable of binding SEMA7A or the epitope designated as SEQ ID NO:2 or 3 or isomer thereof of SEMA7A. In some embodiments, hybridomas herein that can produce such antibodies can be grown in vitro or in vivo using known procedures.

In some embodiments and further to paragraphs [0049]-[00104] above, monoclonal antibodies can be isolated from the culture media or body fluids, by conventional immunoglobulin purification procedures such as ammonium sulfate precipitation, gel electrophoresis, dialysis, chromatography, and ultrafiltration, and the like. In some embodiments, undesired activity, if present, can be removed by, for example but not limited to, running the preparation over adsorbents made of the immunogen attached to a solid phase and eluting or releasing the desired antibodies off the immunogen.

Isolation from an Antibody Library

In certain embodiments and further to paragraphs [0049]-[00105] above, antibodies capable of binding and interfering with the target antigens as described herein can be isolated from an antibody library generated by immunizing an animal and collecting antisera from the animal. This generates a library of unique antibodies or antibody components that can be used to identify antibodies that bind to a specific target antigen (e.g., . SEMA7A. SEQ ID NO: 2 or 3 or isomer thereof of SEMA7A) following routine selection processes as known in the art. In some embodiments, an antibody library can be probed with the target antigen or a fragment thereof and members of the library that are capable of binding to the target antigen can be isolated, typically by retention on a support. In some embodiments, a screening process herein can be performed by multiple rounds (e.g., including both positive and negative selections) to enrich the pool of antibodies capable of binding to the target antigen. In other embodiments, individual clones of the enriched pool can then be isolated and further characterized to identify those having desired binding activity and biological activity. In some embodiments, sequences of the heavy chain and light chain variable domains can also be determined via conventional methodology. There are a number of routine methods known in the art to identify and isolate antibodies capable of binding to the target antigens described herein, including phage display. yeast display, ribosomal display, or mammalian display technology.

In certain embodiments and further to paragraphs [0049]-[00106] above, phage displays herein can use a covalent linkage to bind the protein (e.g., antibody) component to a bacteriophage coat protein. In some embodiments, the linkage can result from translation of a nucleic acid encoding the antibody component fused to the coat protein. In some embodiments, the linkage can include a flexible peptide linker, a protease site, or an amino acid incorporated as a result of suppression of a stop codon. In some embodiments, a bacteriophage displaying the protein component can be grown and harvested using standard phage preparatory methods. (e.g., PEG precipitation from growth media). In some embodiments, after selection of individual display phages, the nucleic acid encoding the selected protein components can be isolated from cells infected with the selected phages or from the phage themselves, after amplification. In some embodiments, individual colonies or plaques can be selected, and then the nucleic acid can be isolated and sequenced. Identifying antibodies or antigen binding proteins having a specific binding affinity

In certain methods and procedures described above and further to paragraphs [0049]-[00107] above, antibodies capable of binding to SEMA7A can be prepared and isolated. In certain embodiments, after antibodies are isolated from the procedures described above for binding to the target antigen, each isolated library member can be tested for its ability to bind to a non-target molecule to evaluate its binding specificity. Examples of non-target molecules include, but are not limited to, streptavidin on magnetic beads, blocking agents such as bovine serum albumin, non-fat bovine milk, soy protein, any capturing or target immobilizing monoclonal antibody, or non-transfected cells which do not express the target. In some embodiments, a high-throughput ELISA screen can be used to obtain the data. In some embodiments, an ELISA screen can also be used to obtain quantitative data for binding of each library member to the target as well as for cross species reactivity to related targets or subunits of the target antigen and also under different condition such as pH 6 or pH 7.5. In accordance with some embodiments herein, non-target and target binding data can be compared (e.g., using a computer and software) to identify library members that specifically bind to the target.

In certain embodiments and further to paragraphs [0049]-[00108] above, after selecting candidate library members that bind to a target, each candidate library member can be further analyzed, e.g., to further characterize its binding properties for the target, e.g. human SEMA7A. SEQ ID NO:2 or 3 or isomer thereof of SEMA7A. In some embodiments, each candidate library member can be subjected to one or more secondary screening assays. In some embodiments, the assay can be for a binding property, a catalytic property, an inhibitory property, a physiological property (e.g., cytotoxicity, renal clearance, or immunogenicity), a structural property (e.g., stability, conformation, oligomerization state) or another functional property. In some embodiments, the same assay can be used repeatedly. but with varying conditions, e.g., to determine pH, ionic, or thermal sensitivities.

In some embodiments and further to paragraphs [0049]-[00109] above, binding proteins can be evaluated using an ELISA assay. In accordance with some embodiments herein, each protein can be contacted to a microtiter plate whose bottom surface has been coated with the target, e.g., a limiting amount of the target. In accordance with some embodiments herein, plates can be washed with buffer to remove non-specifically bound polypeptides. In accordance with these embodiments herein, the amount of the binding protein bound to the target on the plate can be determined by probing the plate with an antibody that can recognize the binding protein, e.g., a tag or constant portion of the binding protein. In accordance with some embodiments herein, the antibody can be linked to a detection system (e.g., an enzyme such as alkaline phosphatase or horse radish peroxidase (HRP) which produces a colorimetric product when appropriate substrates are provided).

In some embodiments and further to paragraphs [0049]-[00110] above, binding proteins can be screened for ability to bind to cells which transiently or stably express and display the target of interest on the cell surface. In some embodiments, SEMA7A binding proteins herein can be fluorescently labeled and binding to SEMA7A in the presence or absence of antagonistic antibody can be detected by a change in fluorescence intensity using flow cytometry e.g., a FACS machine.

In certain embodiments and further to paragraphs [0049]-[00111] above, at any time during the antibody generation process, one or more positive binding antibodies to SEMA7A can be identified. In certain embodiments, certain methods further include sequencing the positive antibodies and expressing the sequence in a recombinant microorganism generated by any method known in the art.

Polynucleotides, vectors, and host cells can be used to prepare an SEMA7A antibody using recombinant technology, as exemplified herein. In certain embodiments. nucleic acids encoding the heavy and light chain of an SEMA7A antibody as described herein can be cloned into one expression vector, each nucleotide sequence being in operable linkage to a suitable promoter. In some embodiments, each of the nucleotide sequences encoding the heavy chain and light chain is in operable linkage to a distinct promoter. In some embodiments, nucleotide sequences encoding the heavy chain and the light chain can be in operable linkage with a single promoter, such that both heavy and light chains are expressed from the same promoter. In some embodiments, when necessary, an internal ribosomal entry site (IRES) can be inserted between the heavy chain and light chain encoding sequences.

In certain embodiments and further to paragraphs [0049]-[00113] above, the expression and/or production of the antibodies in the host cell can be promoted by expressing a leader peptide ahead of the variable region of the heavy or light chain of the antibody. This leader peptide can be optimized for expression in a certain host cell (e.g., . E, coli). One representative sequence that can be used as a leader peptide includes, but is not limited to, a 10-mer or fragment thereof or 12mer, or 14mer or 16mer or 18mer or other larger fragment containing a polypeptide. In some embodiments, a leader sequence can be relatively short such as less than 5 amino acids in length. Leader sequences that are the same length, shorter and longer are known in the art and contemplated of use herein.

In some embodiments and further to paragraphs [0049]-[00114] above, genetically engineered antibodies such as single-chain antibodies can be produced via, e.g., conventional recombinant technology or any methods known in the art. In some embodiments, DNA encoding a monoclonal antibodies specific to a target antigen can be readily isolated and sequenced using conventional procedures (e.g., by using oligonucleotide probes that are capable of binding specifically to genes encoding the heavy and light chains of the monoclonal antibodies). Once isolated, the DNA can be placed into one or more expression vectors, which are then transfected into host cells such as E, coli cells, simian COS cells. Chinese hamster ovary (CHO) cells, or myeloma cells that do not otherwise produce immunoglobulin protein, to obtain the synthesis of monoclonal antibodies in the recombinant host cells. The DNA can then be modified, for example, by substituting the coding sequence for human heavy and light chain constant domains in place of the homologous murine sequences, or by covalently joining to the immunoglobulin coding sequence all or part of the coding sequence for a non-immunoglobulin polypeptide. In some embodiments, genetically engineered antibodies, such as chimeric or hybrid antibodies: can be prepared that have the binding specificity of a target antigen.

In some embodiments, a single-chain antibody herein can be prepared via recombinant technology by linking a nucleotide sequence coding for a heavy chain variable region and a nucleotide sequence coding for a light chain variable region. In some embodiments, a flexible linker is incorporated between the two variable regions. In some embodiments, techniques described for the production of single chain antibodies can be adapted to produce a phage or yeast scFv library and scFv clones specific to SEMA7A can be identified from the library following routine procedures. In some embodiments, positive clones can be subjected to further screening to identify those that bind to SEMAZA or only to SEMA7A or epitope thereof or SEQ ID NO:2 or 3 or isomer thereof of SEMA7A.

In some embodiments, one or more vectors (e.g., expression vectors) having nucleic acids encoding any of the antibodies herein can be introduced into suitable host cells for producing the antibodies. In some embodiments, host cells can be cultured under suitable conditions for expression of the antibody or any polypeptide chain thereof. In some embodiments, antibodies or polypeptide chains thereof can be recovered by the cultured cells (e.g., from the cells or the culture supernatant) via a conventional method, e.g., affinity purification. In some embodiments, polypeptide chains of the antibody herein can be incubated under suitable conditions, for a suitable period of time allowing for production of the antibody.

In some embodiments, methods for preparing an antibody described herein can include a recombinant expression vector that encodes both the heavy chain and the light chain of an SEMA7A antibody, as also described herein. In some embodiments, a recombinant expression vector can be introduced into a suitable host cell (e.g., a dhfr-CHO cell) by a conventional method, e.g., calcium phosphate-mediated transfection. In some embodiments. positive transformant host cells can be selected and cultured under suitable conditions allowing for the expression of the two polypeptide chains that form the antibody, which can be recovered from the cells or from the culture medium. In some embodiments, the two chains recovered from the host cells can be incubated under suitable conditions allowing for the formation of the antibody.

In certain embodiments, two recombinant expression vectors can be provided, one encoding the heavy chain of the SEMA7A antibody (e.g., . SEQ ID NO: 28) and the other encoding the light chain of the SEMA7A antibody (e.g., . SEQ ID NO: 29). In some embodiments, both of the two recombinant expression vectors can be introduced into a suitable host cell (e.g., dhfr-CHO cell) by a conventional method, e.g., calcium phosphate-mediated transfection. In some embodiments, each of the expression vectors can be introduced into a suitable host cells. In some embodiments, positive transformants can be selected and cultured under suitable conditions allowing for the expression of the polypeptide chains of the antibody. In some embodiments, when the two expression vectors are introduced into the same host cells, the antibody produced therein can be recovered from the host cells or from the culture medium. In some embodiments, the polypeptide chains can be recovered from the host cells or from the culture medium and then incubated under suitable conditions allowing for formation of the antibody. In some embodiments, when the two expression vectors are introduced into different host cells, each of them can be recovered from the corresponding host cells or from the corresponding culture media. In some embodiments, two polypeptide chains can then be incubated under suitable conditions for formation of the antibody.

In certain embodiments, standard molecular biology techniques can be used to prepare the recombinant expression vector, transfect the host cells, select for transformants, culture the host cells and recovery of the antibodies from the culture medium. In some embodiments, some antibodies can be isolated by affinity chromatography with a Protein A or Protein G coupled matrix.

In additional embodiments, antibodies herein can be characterized by identifying an epitope or more than one epitope to which the antigen binds, or “epitope mapping.” There are many methods known in the art for mapping and characterizing the location of epitopes on proteins, including, but not limited to, solving the crystal structure of an antibody-antigen complex, competition assays, gene fragment expression assays, and synthetic peptide-based assays. In some embodiments, epitope mapping can be used to determine the sequence, to which an antibody binds and the strength to which it binds a particular epitope.

In certain embodiments, antibodies described herein can have a suitable binding affinity for a target antigen (e.g., SEMA7A) or an epitope thereof. As used herein, “binding affinity” refers to the apparent association constant or KA. The KA is the reciprocal of the dissociation constant (KD). In some embodiments, an antibody described herein can have a binding affinity (KD) of at least about 100 nM, at least about 10 nM, at least about 1 nM, at least about 0.1 nM, or lower for SEMA7A. In some embodiments, an antibody described herein can have a binding affinity (KD) between about 100 nM to about 0.1 nM (e.g., about 100 nM, about 75 nM, about 50 nM, about 25 nM, about 10 nM, about 5 nM, about 1 nM, about 0).75 nM, about 0).5 nM, about 0).25 nM, about 0).1 nM) for SEMA7A. In some embodiments, an antibody described herein can have a binding affinity (KD) between about 50) nM to about 40 nM (e.g., about 50 nM, about 49 nM, about 48 nM, about 47 nM, about 46 nM, about 45 nM, about 44 nM, about 43 nM, about 42 nM, about 41 nM, about 40 nM) for SEMA7A. In some embodiments, an antibody described herein can have a binding affinity (KD) between about 50) nM to about 40 nM (e.g., about 50) nM, about 49 nM, about 48 nM, about 47 nM, about 46 nM, about 45 nM, about 44 nM, about 43 nM, about 42 nM, about 41 nM, about 40 nM) for SEMA7A. In some embodiments, binding affinity (or binding specificity) can be determined by a variety of methods including equilibrium dialysis, equilibrium binding, gel filtration, ELISA, surface plasmon resonance, and/or spectroscopy (e.g., using a fluorescence assay).

In certain embodiments, antibodies described herein can block SEMA7A pro-tumor activities. In certain embodiments, antibodies described herein can reduce or eliminate SEMA7A and/or SEMA7A activities by about 5% to about 99% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%) compared to an untreated subject or other control.

In certain embodiments, antibodies described herein can have neutralizing activity against SEMA7A. In some embodiments, antibodies described herein can have neutralizing activity against SEMA7A with IC50 less than about 100, 125, 150, 175 or 200 ng/ml. In some embodiments, antibodies described herein can have neutralizing activity against

SEMATA with IC50 ranging from about 0).1 ng/ml to about 200 ng/ml (e.g., about 0.1 ng/ml. about 0).25 ng/ml, about 0).5 ng/ml, about 1 ng/ml, about 2.5 ng/ml, about 5 ng/ml, about 7.5 ng/ml, about 10 ng/ml, about 25 ng/ml, about 50 ng/ml, about 75 ng/ml, about 100 ng/ml. about 125 ng/ml, about 150 ng/ml).

In certain embodiments, the heavy chain of any of the antibodies disclosed herein can further include a heavy chain constant region (CH) or a fragment thereof (e.g., . CHI. CH2. CH3, or a combination thereof). In some embodiments, a heavy chain constant region for use herein can be of any suitable origin, e.g., human, mouse, rat, or rabbit. In some embodiments, alternatively or in addition, a light chain of any of the antibodies disclosed herein can further include a light chain constant region (CL), which can be any CL known in the art. In some embodiments, a CL can be a kappa light chain. In some embodiments, a CL can be a lambda light chain. Antibody heavy and light chain constant regions are well known in the art. In some embodiments, an illustrative heavy chain including the heavy chain constant region is provided herein as SEQ ID NO: 28. In some embodiments, an illustrative light chain including the light chain constant region is provided herein as SEQ ID NO: 29.

In some embodiments, a linker can be incorporated between the two variable regions disclosed herein such as a flexible peptide linker, a rigid peptide linker, a cleavable peptide linker, or any combination thereof. In accordance with these embodiments, a peptide linker incorporated between the two variable regions herein can be at least one amino acid. In some embodiments, a peptide linker incorporated between the two variable regions disclosed herein can be about 1 amino acid to about 50 amino acids (e.g., about 1, about 2, about 3, about 4, about 5, about 6, about 7, about 8, about 9, about 10), about 11, about 12, about 13, about 14. about 15, about 16, about 17, about 18, about 19, about 20, about 21, about 22, about 23. about 24, about 25, about 26, about 27, about 28, about 29, about 30), about 32, about 34. about 36, about 38, about 40, about 42, about 44, about 46, about 48, up to 50 amino acids). In some embodiments, a scFv antibody herein can include a VH fragment and a VL fragment. which can be linked by a peptide linker such as a flexible peptide linker.

In some embodiments, a scFv antibody herein can be in the VH VL orientation (from N-terminus to C-terminus). In some embodiments, a scFv antibody disclosed herein can be in the VL VH orientation (from N-terminus to C-terminus).

In some embodiments, isolated monoclonal antibodies disclosed herein can be a human antibody or a humanized antibody. Humanized antibodies refer to forms of non-human (e.g., murine) antibodies that are specific chimeric immunoglobulins, immunoglobulin chains, or antigen-binding fragments thereof that contain minimal sequence derived from non-human immunoglobulin.

In certain embodiments, the expression and/or production of the antibodies in the host cell can be promoted by expressing a leader peptide ahead of the variable region of the heavy or light chain of the antibody. In some embodiments, a leader peptide can be optimized for expression in a certain host cell (e.g., . E, coli).

In some embodiments, genetically engineered antibodies such as single-chain antibodies can be produced via, e.g., conventional recombinant technology or any methods known in the art. In some embodiments, DNA encoding a monoclonal antibodies specific to a target antigen can be readily isolated and sequenced using conventional procedures (e.g., by using oligonucleotide probes that are capable of binding specifically to genes encoding the heavy and light chains of the monoclonal antibodies). Once isolated, the DNA can be placed into one or more expression vectors, which are then transfected into host cells such as E, coli cells, simian COS cells. Chinese hamster ovary (CHO) cells, or myeloma cells that do not otherwise produce immunoglobulin protein, to obtain the synthesis of monoclonal antibodies in the recombinant host cells. The DNA can then be modified, for example, by substituting the coding sequence for human heavy and light chain constant domains in place of the homologous murine sequences, or by covalently joining to the immunoglobulin coding sequence all or part of the coding sequence for a non-immunoglobulin polypeptide. In some embodiments, genetically engineered antibodies, such as chimeric or hybrid antibodies: can be prepared that have the binding specificity of a target antigen.

In some embodiments, a single-chain antibody herein can be prepared via recombinant technology by linking a nucleotide sequence coding for a heavy chain variable region and a nucleotide sequence coding for a light chain variable region. In some embodiments, a linker can be incorporated between the two variable regions. In some embodiments, techniques described for the production of single chain antibodies can be adapted to produce a phage or yeast scFv library and scFv clones specific to the SEMA7A epitope can be identified from the library following routine procedures. In some embodiments, positive clones can be subjected to further screening.

In some embodiments, one or more vectors (e.g., expression vectors) having nucleic acids encoding any of the antibodies herein can be introduced into suitable host cells for producing the antibodies. In some embodiments, host cells can be cultured under suitable conditions for expression of the antibody or any polypeptide chain thereof. In some embodiments, antibodies or polypeptide chains thereof can be recovered by the cultured cells (e.g., from the cells or the culture supernatant) via a conventional method, e.g., affinity purification. In some embodiments, polypeptide chains of the antibody herein can be incubated under suitable conditions for a suitable period of time allowing for production of the antibody.

In some embodiments, methods for preparing an antibody described herein can include a recombinant expression vector that encodes both the heavy chain and the light chain of an antibody that binds to a SEMA7A epitope (e.g., . SEQ ID NO:2), as described herein. In some embodiments, a recombinant expression vector can be introduced into a suitable host cell (e.g., a dhfr-CHO cell) by a conventional method, e.g., calcium phosphate-mediated transfection. In some embodiments, positive transformant host cells can be selected and cultured under suitable conditions allowing for the expression of the two polypeptide chains that form the antibody, which can be recovered from the cells or from the culture medium. In some embodiments, the two chains recovered from the host cells can be incubated under suitable conditions allowing for the formation of the antibody.

In certain embodiments, two recombinant expression vectors can be provided, one encoding the heavy chain of a disclosed antibody and the other encoding the light chain of the disclosed antibody. In some embodiments, both of the recombinant expression vectors can be introduced into a suitable host cell (e.g., dhfr-CHO cell) by a conventional method, e.g., calcium phosphate-mediated transfection. In some embodiments, each of the expression vectors can be introduced into a suitable host cell. In some embodiments, positive transformants can be selected and cultured under suitable conditions allowing for the expression of the polypeptide chains of the antibody. In some embodiments, when the two expression vectors are introduced into the same host cells, the antibody produced therein can be recovered from the host cells or from the culture medium. In some embodiments, the polypeptide chains can be recovered from the host cells or from the culture medium and then incubated under suitable conditions allowing for formation of the antibody. In some embodiments, when the two expression vectors are introduced into different host cells, each of them can be recovered from the corresponding host cells or from the corresponding culture media. In some embodiments, two polypeptide chains can then be incubated under suitable conditions for formation of the antibody.

In certain embodiments, standard molecular biology techniques can be used to prepare the recombinant expression vector, transfect the host cells, select for transformants. culture the host cells, and recover the antibodies from the culture medium. In some embodiments, some antibodies can be isolated by affinity chromatography with a Protein A or Protein G coupled matrix.

In additional embodiments, antibodies herein can be characterized by identifying an epitope or more than one epitope to which the antigen binds, or “epitope mapping.” There are many methods known in the art for mapping and characterizing the location of epitopes on proteins, including, but not limited to, solving the crystal structure of an antibody-antigen complex, competition assays, gene fragment expression assays, and synthetic peptide-based assays. In some embodiments, epitope mapping can be used to determine the sequence, to which an antibody binds.

### Therapeutic and Diagnostic Applications

In some embodiments and further to paragraphs [0049]-[00136] above. SEMA7A inhibition using antibodies or fusion polypeptides and/or fragments thereof, can be used to treat conditions characterized by SEMA7A expression or overexpression in a subject. Conditions related to pathological overexpression of SEMA7A include myocardial infarction (heart attack), cancer or pre-malignant disease (e.g., benign breast disease, atypical hyperplasia or carcinoma in situ), inflammatory conditions such as arthritis, colitis, IBS, IBD, airway inflammation and Gucher disease, fibrosis such as liver or lung fibrosis, or multiple sclerosis, or other health condition where SEMA7A is expressed or over-expressed and/or contributes to adverse health conditions in a subject. In accordance with these embodiments. use of one or more antibody or monoclonal antibody (e.g., against SEQ ID NO:2 or 3 or isomer thereof) disclosed herein can be used to treat a subject having a condition characterized by overexpression of SEMA7 to mitigate one or more symptoms or treat the health condition in a subject. In some embodiments, antibodies or monoclonal antibody treatments disclosed herein can be used to treat a subject daily, every other day, every three days, bi-weekly, every week, bi-monthly or other appropriate treatment regimen. In other embodiments, a subject can be treated when SEMA7A is detected in a blood or other sample which can include a single or multiple treatments with antibodies disclosed herein. In other embodiments, other markers can be measured besides SEMA7A in one or more samples such as a blood or tumor biopsy. In accordance with these embodiment, one or more samples such as a blood or tumor biopsy can be assessed for levels of one or more of alpha-6 integrin. FN. COX-2 and PD-L1 to tailor treatment of the subject to reduce, eliminate, or prevent a condition characterized by overexpression of SEMA7A (e.g., cancer, myocardial infarction, inflammation etc.)

In some embodiments and further to paragraphs [0049]-[00137] above. SEMA7A inhibition using antibodies or fusion polypeptides and/or fragments thereof, can reduce tumor growth and expansion, induce tumor cell killing and/or reduce tumor progression and metastasis. Cancers to be treated contemplated herein can include solid or non-solid tumors such as cancers of the blood. Solid tumors can include, but are not limited to, breast, pancreatic, brain, mesothelioma, lung, thyroid, stomach, liver, kidney, ovarian, prostate, breast or other solid tumor. Non-solid tumors include, but are not limited to, leukemias and other known non-solid tumors and metastasizing tumors expressing SEMA7A or overexpressing SEMA7A. In accordance with these embodiments, use of one or more antibody or monoclonal antibody (e.g., against SEQ ID NO:2 or 3 or isomer thereof) disclosed herein can be used to treat a subject having cancer to reduce or prevent expansion, migration, to shrink a tumor and/or to reduce metastasis of the cancer in a subject. In some embodiments, antibodies or monoclonal antibody treatments disclosed herein can be used to treat a subject daily, every other day, every three days, bi-weekly, every week, bi-monthly or other appropriate treatment regimen. In other embodiments, a subject can be treated when SEMA7A is detected in a blood or other sample which can include a single or multiple treatments with antibodies disclosed herein. In other embodiments, other markers can be measured besides SEMA7A in one or more samples such as a blood or tumor biopsy. In accordance with these embodiment, one or more samples such as a blood or tumor biopsy can be assessed for levels of one or more of alpha-6 integrin. FN. COX-2 and PD-L1 in order to tailor treatment of the subject to reduce, eliminate or prevent a condition such as cancer (e.g., breast cancer or PPBC etc.)

In other embodiments and further to paragraphs [0049]-[00138] above, one or more antibodies disclosed herein or any other SEMA7A antibody available can be used to determine prognosis of a subject having a health condition. In accordance with these embodiments, a SEMA7A antibody or an antibody or monoclonal antibody disclosed herein can be used to measure levels or presence of SEMATA in a sample (e.g., a fluid sample) from a subject and prognosis of the subject can be determined. In some embodiments, a poor or positive prognosis can be assessed based on level of SEMA7A in the tissue, blood or other fluid sample of a subject having a heart attack or other condition such as cancer, chronic inflammation, or fibrosis. In accordance with these embodiments, a SEMA7A concentration in a blood sample of about 0, 1 ng/ml, or about 0.5 ng/ml or about 1.0 ng/ml and above can indicate a poor prognosis and intervention should be undertaken or course of treatment modified or increased. In some embodiments, a level of about 1 ng/ml in a fluid sample (e.g. blood) of a subject corresponds to a poor prognosis in a subject for a health condition including, but not limited to, a subject having a heart attack, a subject having cancer or pre-malignant disease (e.g, benign breast disease, atypical hyperplasia or carcinoma in situ), a subject having an inflammatory condition, fibrosis or other health condition where SEMA7A is expressed or over-expressed and/or contributes to adverse health conditions in a subject. In some embodiments, where at risk levels of SEMATA are observed, a SEMA7A antibody or other inhibitor (e.g., monoclonal antibody) can be administered as a therapeutic intervention to prevent or reduce progression of the health condition. In certain embodiment, a SEMA7A polyclonal antibody or monoclonal antibody against SEQ ID NO:2 or 3 or isomer thereof is administered as a therapeutic intervention to prevent or reduce progression of the health condition such as cancer. In some embodiments, a candidate dose is an equivalent dose of about 5 to about 20 ug per dose in comparison to studies disclosed herein or a dose known in the art for inhibiting SEMA7A adverse effects, or a standard monoclonal antibody concentration known in the art.

In certain embodiments and further to paragraphs [0049]-[00139] above, antibodies (e.g, monoclonal antibodies) disclosed herein directed to bind SEMA7A can be used to treat health conditions associated with expression or over-expression of SEMA7A or SEMA7A activity. In accordance with these embodiments, the health condition to be treated includes, but is not limited to, health conditions concerning neurological development, neurological conditions, wound healing, bone homeostasis conditions, angiogenesis issues, pro-inflammatory cytokine release and regulation-related conditions, dendritic cell-related adverse effects on adhesion and motility, fibrosis-related conditions and effects on anti-inflammatory IL-10 release effects as well as effects on tumor metastasis and progression where inhibition of SEMA7A expression and/or activities treats the condition. In certain embodiments, antibodies disclosed herein can be used to treat one or more of these conditions to alleviate the condition.

In some embodiments and further to paragraphs [0049]-[00140] above, cancer cells or related growths in a subject can be treated with SEMA7A antibodies and/or other inhibitory molecules or technologies disclosed herein to reduce tumor progression, reduce or eliminate cancer stem cells or cancer stem cell metastasis, reduce or inhibit metastasis of other tumor cells, shrink tumors, induce tumor cell death, reduce tumor expansion, reduce lymphatic invasion and/or reduce lymphogenous metastasis (e.g, reduce infiltration or metastasis of tumor cells into lymph nodes) In other embodiments, antibodies disclosed herein can be used to reduce recruitment of suppressive immune cells such as macrophages and to reduce fibrillar collagen deposition.

In yet other embodiments and further to paragraphs [0049]-[00141] above. SEMA7A presence or levels can be detected in a tumor or samples from a subject to assess SEMA7A involvement in pro-tumor effects or presence in a pro-tumor environment. In accordance with these embodiments, if SEMA7A is present or the level has reached a predetermined threshold (e.g., 1 ng/ml), the tumor can be treated with one or more SEMA7A inhibitors to reduce tumor expansion, shrink tumor size and/or reduce tumor metastasis. These treatments can be used alone or in combination with other anti-cancer, anti-breast cancer or other treatments.

In other embodiments and further to paragraphs [0049]-[00142] above, inhibitors of SEMA7A expression or activities can include any SEMA7A inhibitor or antibody or peptide directed to block or interfere with SEMA7A expression or activities disclosed herein. In certain embodiments, inhibitors of SEMA7A expression or activities can include, but are not limited to, antibodies, anti-sense RNA, siRNA, an agent that targets SEMA7A or use of any editing technology such as Crispr or other gene editing technology capable of genetically manipulating SEMA7A to edit SEMA7A by completely removing it or editing it to reduce its pro-tumor activities, for example. It is contemplated that any of these methods and systems can be used to treat a subject having cancer with SEMA7A involvement.

In certain embodiments and further to paragraphs [0049]-[00143] above, a nucleic acid molecule disclosed herein can be used to modulate gene expression of SEMA7A in a targeted cell. As used herein, the term “genetically modified” refers to manipulation of a cell genome using genetic engineering techniques. Non-limiting examples of genetic engineering techniques that can be used to modulate gene expression of SEMA7A in a target cell can include chemical mutagenesis, x-ray mutagenesis, recombinant DNA techniques, virus-mediated delivery of DNA, and gene editing. Some examples of gene editing methods include, but are not limited to, CRISPRs, TALENs and Zinc Finger Nucleases.

### Pharmaceutical Compositions

In certain embodiments and further to paragraphs [0049]-[00144] above, pharmaceutical compositions are disclosed herein. In accordance with these embodiments, pharmaceutical compositions can include a pharmaceutically acceptable carrier combined with a SEMA7A antibody or fragment or peptide-binding construct disclosed herein. Pharmaceutically acceptable excipients (carriers) can be any excipient including those known in the art.

In certain embodiments and further to paragraphs [0049]-[00145] above, the pharmaceutical compositions can further include a non-specific innate immune response stimulator mixture or composition or other immunomodulatory agent for enhancing an immune response. In accordance with these embodiments, the non-specific innate immune response stimulator can elicit both a cell-mediated immune response and a humoral immune response.

In other embodiments and further to paragraphs [0049]-[00146] above, pharmaceutical compositions described herein can further include an anti-microbial agent, a chemotherapeutic agent, and/or other anti-cancer therapeutic or antibody. In accordance with these embodiments, the anti-microbial agent can, in an example, be an anti-viral, bactericidal agent, anti-fungal, or anti-bacterial agent. For example, the anti-microbial agent can be an anti-bacterial agent (antibiotic) such as doxycycline or another antibiotic such as a general antibiotic. In other embodiments, targeted anti-cancer agents such as those that target receptor tyrosine kinases, hormone receptors, anti-apoptotic mechanisms, cell cycle inhibitors, metabolism, inflammation, statins and additional agents can in advance of or at the time of or after administering SEMA7A antibodies or inhibitors disclosed herein.

Pharmaceutically acceptable carriers or excipients suitable for the compositions described herein are well known to one of skill in the art of use for preserving and delivering antibodies or antibody fragments to any mammalian subject including humans and other mammals. It is contemplated that any pharmaceutically acceptable agent can be used in compositions disclosed herein.

In certain embodiments and further to paragraphs [0049]-[00148] above, the pharmaceutical compositions to be used in the present methods can include pharmaceutically acceptable carriers, excipients, or stabilizers in the form of lyophilized formulations or aqueous solutions. In some embodiments, acceptable carriers, excipients, or stabilizers are nontoxic to recipients at the dosages and concentrations used, and can include buffers such as phosphate, citrate, and other organic acids: antioxidants including ascorbic acid and methionine: preservatives (such as octadecyldimethylbenzyl ammonium chloride; hexamethonium chloride: benzalkonium chloride, benzethonium chloride: phenol, butyl or benzyl alcohol: alkyl parabens such as methyl or propyl paraben: catechol: resorcinol: cyclohexanol: 3-pentanol; and m-cresol): low molecular weight (less than about 10 residues) polypeptides: proteins, such as serum albumin, gelatin, or immunoglobulins: hydrophilic polymers such as polyvinylpyrrolidone: amino acids such as glycine, glutamine, asparagine, histidine, arginine, or lysine: monosaccharides, disaccharides, and other carbohydrates including glucose, mannose, or dextrans: chelating agents such as EDTA: sugars such as sucrose, mannitol, trehalose or sorbitol: salt-forming counter-ions such as sodium; metal complexes (e.g. Zn-protein complexes); and/or non-ionic surfactants such as TWEEN™. PLURONICS™ or polyethylene glycol (PEG).

In some embodiments and further to paragraphs [0049]-[00149] above, the pharmaceutical composition described herein can have liposomes containing the antibodies (or the encoding nucleic acids). In some embodiments, liposomes for use herein can be generated by the reverse phase evaporation method with a lipid composition having phosphatidylcholine, cholesterol and PEG-derivatized phosphatidylethanolamine (PEG-PE). In some embodiments, liposomes for use herein can be extruded through filters of defined pore size to yield liposomes with the desired diameter. In some embodiments, antibodies, or the encoding nucleic acid(s) herein, can be entrapped in microcapsules prepared, for example, by coacervation techniques or by interfacial polymerization, for example. hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions.

In other embodiments and further to paragraphs [0049]-[00150] above, the pharmaceutical compositions described herein can be formulated in sustained-release format. In some embodiments, pharmaceutical compositions herein to be used for in vivo administration must be sterile. In some embodiments, this can be readily accomplished by, for example, filtration through sterile filtration membranes. In some embodiments, therapeutic antibody compositions can be placed into a container having a sterile access port, for example, an intravenous solution bag or vial having a stopper pierceable by a hypodermic injection needle.

In certain embodiments, pharmaceutical compositions described herein can be in unit dosage forms such as tablets, pills, capsules, powders, granules, solutions or suspensions, or suppositories, for oral, parenteral, or rectal administration, or administration by inhalation or insufflation.

In some embodiments, emulsion compositions disclosed herein can be those prepared by mixing an antibody with Intralipid™ or the components thereof (soy bean oil, egg phospholipids, glycerol and water).

In some embodiments and further to paragraphs [0049]-[00153] above, pharmaceutical compositions disclosed herein can include formulations for inhalation or intranasal administration include solutions and suspensions in pharmaceutically acceptable, aqueous or organic solvents, or mixtures thereof, and powders. In some embodiments, liquid or solid compositions herein can contain suitable pharmaceutically acceptable excipients as set out above. In other embodiments, the compositions can be administered by the oral or nasal respiratory route for local or systemic effect.

In some embodiments and further to paragraphs [0049]-[00154] above, compositions can include sterile pharmaceutically acceptable solvents to be nebulized by use of gases. In some embodiments, nebulized solutions herein can be breathed directly from the nebulizing device, or the nebulizing device can be attached to a face mask, tent or intermittent positive pressure breathing machine. In some embodiments, solution, suspension, or powder compositions herein can be administered, preferably orally or nasally, from devices which deliver the formulation in an appropriate manner.

In some embodiments and further to paragraphs [0049]-[00155] above, concentrations of antibodies, monoclonal antibodies or fragments thereon disclosed herein can be a pre-determined concentration or a standard concentration. In some embodiments, concentrations of antibodies disclosed herein can be in a concentration of about 1-1000 mg/ml. 1-500 mg/ml, 1-250 mg/ml, 1-200 mg/ml, 1-150 mg/ml, 1-100 mg/ml, 1-75 mg/ml, or 1-50 mg/ml, or 0).1 to 100 micrograms or other suitable concentration. In some embodiments, the antibody is formulated to a concentration of about 30 mg/ml. In some embodiments, the antibody is lyophilized. In some embodiments, the antibody is diluted in a suitable solution to a suitable concentration prior to administration (e.g., in a therapeutic application described below). In some embodiments, a single or multiple dose formulation can be created for single or multiple administrations to a subject in need thereof.

### Methods of Use-Therapeutic Applications

In certain embodiments, and further to paragraphs [0049]-[00156] above, the condition to be treated by compositions disclosed herein is any condition characterized by SEMA7A upregulation or expression. Conditions related to pathological overexpression of SEMA7A include myocardial infarction (heart attack), cancer or pre-malignant disease (e.g., benign breast disease, atypical hyperplasia or carcinoma in situ), inflammatory conditions such as arthritis (e.g., RA), colitis. IBD. IBD, airway inflammation, and Gaucher disease, liver or lung fibrosis, multiple sclerosis, or other health condition where SEMA7A is expressed or over-expressed and/or contributes to adverse health conditions in a subject. In accordance with these embodiments, use of one or more antibody or monoclonal antibody (e.g., against SEQ ID NO:2 or 3 or isomer thereof) disclosed herein can be used to treat a subject having a condition characterized by overexpression of SEMA7 to mitigate one or more symptoms of the condition or treat the underlying condition. In some embodiments, antibodies or monoclonal antibody treatments disclosed herein can be used to treat a subject daily, every other day, every three days, bi-weekly, every week, bi-monthly or other appropriate treatment regimen. In other embodiments, a subject can be treated when SEMA7A is detected in a blood or other sample which can include a single or multiple treatments with antibodies disclosed herein. In other embodiments, other markers can be measured besides SEMA7A in one or more samples such as a blood or tumor biopsy. In accordance with these embodiment, one or more samples such as a blood or tumor biopsy can be assessed for levels of one or more of alpha-6 integrin. FN. COX-2 and PD-L1 to tailor treatment of the subject to reduce, eliminate or prevent a condition characterized by overexpression of SEMA7A (e.g., cancer, myocardial infarction, inflammation, etc).

In certain embodiments and further to paragraphs [0049]-[00157] above, the cancer to be treated by compositions disclosed herein is a solid tumor. In other embodiments, the solid tumor can be a head, neck, brain, lung, breast, liver, kidney, ovarian, testicular, prostate or colon tumor or other solid tumor. In some embodiments, administering compositions disclosed herein can be by direct injection into the tumor of the subject. In certain embodiments, the method can include systemic administration of the composition.

In certain embodiments and further to paragraphs [0049]-[00158] above, a subject having cancer can be identified by routine medical examination, e.g., laboratory tests, organ functional tests. CT scans, or ultrasounds. In some embodiments, the subject to be treated by the method described herein can have undergone or is subjecting to an anti-cancer therapy, for example, chemotherapy, radiotherapy, immunotherapy, or surgery. In certain embodiments, a subject can be diagnosed with the presence of SEMA7A and antibodies or fragments or peptides directed to bind and neutralize SEMA7A activity can be used to treat or reduce onset of cancer or relapse of cancer in the subject.

In some embodiments and further to paragraphs [0049]-[00159] above, treatment efficacy for a target disease/disorder can be assessed by methods well-known in the art. In accordance with these embodiments, cancer progression or remission can be analyzed by any method known in the art in order to adjust treatment regimens as needed.

### Kits

In certain embodiments and further to paragraphs [0049]-[00160] above, kits are provided for storage, transport and use in treating or alleviating a target disease, such an immunosuppressed condition or cancer as described herein. In some embodiments, kits can include one or more containers. In other embodiments, kits disclosed herein contain at least one anti-SEMA7A antibody, fragment thereof, or composition thereof. In certain embodiments, kits can include monoclonal antibodies disclosed herein as a concentrate, in a storage medium or in a pharmaceutically acceptable formulation of use to treat a subject. Single dose up to multiple dose formulations are contemplated to be part of a kit.

In some embodiments and further to paragraphs [0049]-[00161] above, kits can include at least one antibody disclosed herein and at least one container in accordance with any of the methods described herein. In some embodiments, instructions can be included and can contain a description of administration of the SEMA7A antibody, and optionally, a second therapeutic agent, to treat, delay the onset, or alleviate a target disease as those described herein. In other embodiments, kits can further include a description of selecting an individual suitable for treatment based on identifying whether that individual has the target disease, e.g., applying the diagnostic method as described herein. In still other embodiments, instructions can be provided and have a description of administering an antibody to an individual at risk of the target disease. In some embodiments, kits can include at least one composition disclosed herein and at least one container for storage and/or transport of the at least one composition for later use.

### EXAMPLES

The following examples are included to illustrate certain embodiments. It should be appreciated by those of skill in the art that the techniques disclosed in the examples which follow represent techniques discovered to function well in the practice of the claimed methods, compositions, and apparatus. However, those of skill in the art should, in light of the present disclosure, appreciate that changes can be made in some embodiments which are disclosed and still obtain a like or similar result without departing from the spirit and scope of embodiments of the inventions.

### Example 1

In one exemplary method, peptide fragments of Semaphorin 7A (SEMA7A, referred to also as CD108, or Sema KI or Sema L) were generated. SEMA7A is a member of the Semaphorin family of axon guidance molecules and, when expressed on human erythrocytes, is the John Milton Hagen (JMH) blood group antigen. SEMA7A is an ˜80 kDa membrane-anchored glycoprotein that contains an RGD integrin interaction motif within its full-length amino acid sequence where a representative sequence is referenced below:

HUMAN SEMA7A
(SEQ ID NO: 1)
MTPPPPGRAAPSAPRARVPGPPARLGLPLRLRLLLLLWAAAASAQGHLR
SGPRIFAVWKGHVGQDRVDFGQTEPHTVLFHEPGSSSVWVGGRGKVYLF
DFPEGKNASVRTVNIGSTKGSCLDKRDCENYITLLERRSEGLLACGTNA
RHPSCWNLVNGTVVPLGEMRGYAPFSPDENSLVLFEGDEVYSTIRKQEY
NGKIPRFRRIRGESELYTSDTVMQNPQFIKATIVHQDQAYDDKIYYFFR
EDNPDKNPEAPLNVSRVAQLCRGDQGGESSLSVSKWNTFLKAMLVCSDA
ATNKNFNRLQDVFLLPDPSGQWRDTRVYGVFSNPWNYSAVCVYSLGDID
KVFRTSSLKGYHSSLPNPRPGKCLPDQQPIPTETFQVADRHPEVAQRVE
PMGPLKTPLFHSKYHYQKVAVHRMQASHGETFHVLYLTTDRGTIHKVVE
PGEQEHSFAFNIMEIQPFRRAAAIQTMSLDAERRKLYVSSQWEVSQVPL
DLCEVYGGGCHGCLMSRDPYCGWDQGRCISIYSSERSVLQSINPAEPHK
ECPNPKPDKAPLQKVSLAPNSRYYLSCPMESRHATYSWRHKENVEQSCE
PGHQSPNCILFIENLTAQQYGHYFCEAQEGSYFREAQHWQLLPEDGIMA
EHLLGHACALAASLWLGVLPTLTLGLLVH

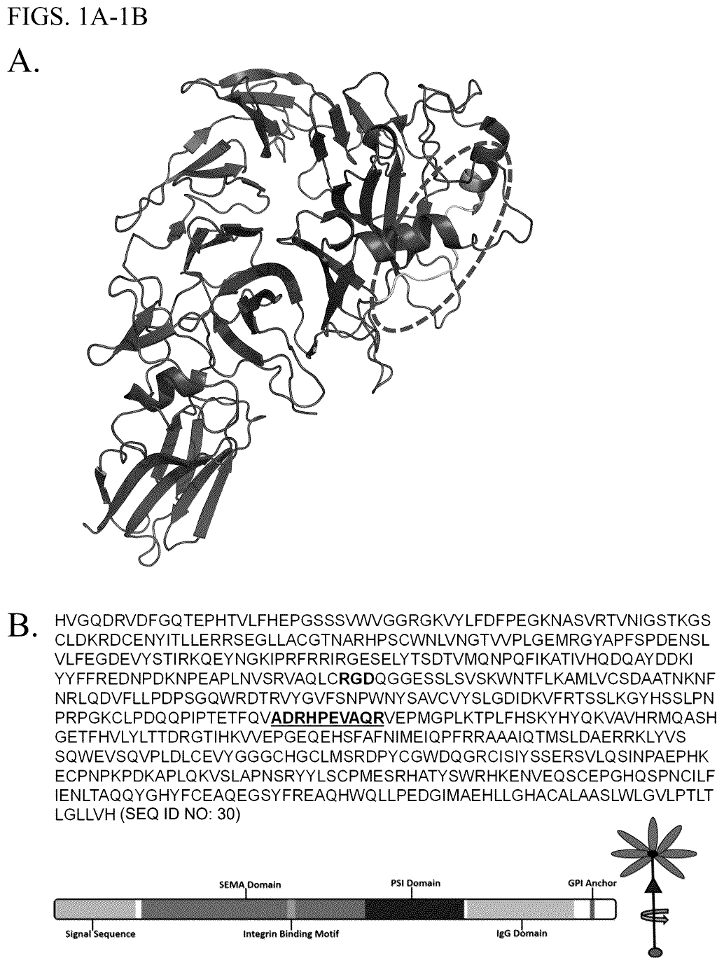

Using SEQ ID NO: 1 as a representative sequence of SEMA7A, a 10 amino acid peptide was identified of use in constructs and compositions disclosed herein. This peptide was identified having only exhibited sequence homology to human SEMA7A and no other known peptide. The peptide was synthesized and purified. The 10 amino acid SEMA7A peptide corresponds to amino acid residues 381-392 of the human full-length SEMA7A (SEQ ID NO: 1) and has an amino acid sequence of: ADRHPEVAQR (SEQ ID NO: 2). FIG. 1A illustrates a 2D structure of a human SEMA7A protein where the location of the selected 10 amino acid sequence used to design the peptides herein is indicated by a loop. FIG. 1B illustrates the context of the peptide (SEQ ID NO: 2, bold and underlined) within the domains of SEMA7A (SEQ ID NO: 30) and its proximity to the beta-1 integrin binding site (bolded). In brief, state-of-the-art automated instrumentation for solid-phase peptide synthesis was used to synthesize the peptides of interest using 3-maleimido-benzoic acid-OSu (Boc)-chemistry. The resulting peptides were then subjected to peptide purification by reverse-phase HPLC followed by peptide identification by mass spectrometry (LC-MS). In addition to the 10 amino acid SEMA7A peptide, a peptide for later conjugation was also synthesized (e.g., CGGADRHPEVAQR: SEQ ID NO: 3). It is noted that other conjugated constructs or polypeptide fragments or polynucleotide related thereto can be of use in constructs and compositions for generating antibodies disclosed herein having at least 75% up to 100% homology to the 10-mer and of longer or shorter lengths such as a 5-mer or a 15-mer. 20)-mer or other suitable size.

### Example 2

In another exemplary method, the 10 amino acid SEMA7A peptides designed and synthesized according to the exemplary methods of Example 1 were used to generate multiple antibodies having affinity for SEMA7A. In some exemplary examples, a peptide represented by SEQ ID NO: 2 is used to generate polyclonal and monoclonal antibodies. In other exemplary examples, a peptide represented by SEQ ID NO: 3 is used to generate a peptide-protein conjugate to produce polyclonal and monoclonal antibodies against the 10 amino acid SEMA7A peptide. Because peptides alone can be too small to elicit a sufficient immune response, conjugation of the 10 amino acid SEMA7A peptide to a carrier protein can yield a more robust immune response. Therefore, any conjugate is contemplated herein to be used to bind to the peptides of interest and used to generate robust antibodies against SEMA7A. In certain examples, because some antibodies were raised against the peptide, the conjugation amino acid residues, and the carrier protein, choice of carrier protein as a conjugate during immunization differs from that to be used in the desired final assay. For example. KLH conjugates are used to immunize for antibodies when BSA will be used in the end-point assays. In some exemplary examples, a peptide represented by the sequence of SEQ ID NO: 3 is conjugated to the carrier protein (e.g., . BSA, bovine serum albumin) but other suitable carrier proteins are also available and contemplated. In other exemplary examples, a peptide represented by the sequence of SEQ ID NO: 3 or SEQ ID NO. 2 is conjugated to the carrier protein KLH (keyhole limpet hemocyanin), for example.

In some exemplary methods, the 10 amino acid SEMA7A peptide and the 10 amino acid SEMA7A peptide-conjugated to a carrier protein were used to generate monoclonal antibodies. In brief, the peptides described herein were utilized to immunize mice which generated hybridomas from two different mice having serum that exhibited high level specificity for purified SEMA7A and for SEMA7A in human breast tumor cells and in human breast tissues. The hybridoma cultures generated from the immunized mice were screened, expanded and the selected clones were selected for production and purification of mouse monoclonal antibodies for SEMA7A.

In another exemplary method, the 10 amino acid SEMA7A peptide and the 10 amino acid SEMA7A peptide-conjugated to a carrier protein are used to generate polyclonal antibodies. In brief, polyclonal antibodies are produced by injecting one of the peptides herein into an animal (e.g., a rabbit). After being injected with the peptides to elicit a primary immune response, the animal is given a second, and even a third injection of the peptide to produce higher titers of antibodies against the 10 amino acid SEMA7A peptide.

Both polyclonal and monoclonal antibodies to the SEMA7A peptide were characterized. In brief, human SEMA7A protein is produced and purified from human breast cancer cells. Purified SEMA7A, alongside whole cell lysates from breast cancer cells with stable knockdown or overexpression of SEMA7A, provides samples for demonstrating binding specificity of the antibodies generated herein via Western blot and/or dot plot. Additionally, tissue lysates from wild-type and SEMA7A knockout mice are subjected to Western blot analysis using polyclonal and monoclonal antibodies to the SEMA7A peptide for validation of specificity. As further validation of specificity, polyclonal and monoclonal antibodies to the SEMA7A peptides were used to perform immunohistochemistry (IHC) on formalin-fixed human tissues collected from healthy breast tissue and cancerous biopsies.

### Example 3

In another exemplary method, a threshold for SEMA7A levels in blood as a biomarker for breast cancer prognosis in humans has been established. Mammary tissue biopsies and matched blood samples from age-matched, cancer-free, nulliparous, and postpartum women were collected. Mammary tissue biopsies and matched blood samples are analyzed with IHC and ELISA, respectively, to determine baseline SEMA7A levels in normal breast tissue and blood. Blood was collected from healthy donors to help establish base blood SEMA7A levels in healthy individuals compared to breast cancer patients. To determine the relationship between SEMA7A presence and/or levels and breast cancer prognosis, matched blood samples were compared to tumors from both PPBC and nulliparous patients where the patients contributing to the samples in the study also provided complete reproductive history, clinicopathologic annotation, updated clinical outcomes for local and distant metastasis, and treatment information. Using SEMA7A antibodies capable of binding any SEMA7A, over 120 mammary tissue and tumor samples were stained and quantified for SEMA7A. After identifying patients with known levels of SEMA7A expression in their tissues, matched serum was tested with SEMA7A ELISA to correlate SEMA7A levels to those in the tumor. Both tumor and blood SEMA7A levels were compared to patient history to evaluate whether SEMA7A expression levels are predictive of prognosis. SEMA7A antibodies used in ELISA assays detected SEMA7A in the blood. Data demonstrated that detecting as low as 1 ng/mL of SEMA7A in the blood via ELISA was associated with having >30 percent expression in the tumor tissue, which in this example, was critical for predicting recurrence in breast cancer prognosis. In certain cases, patients having a history of breast cancer or currently having breast cancer identified as further having SEMA7A or elevated levels of SEMA7A in their samples can indicate the need for immediate therapeutic intervention and/or alterative intervention compared to standard cancer therapies (data not shown).

These exemplary results demonstrating antibodies detecting SEMA7A in the blood provide a diagnostic tool for assessing whether this high-risk population, patients with SEMA7A+breast cancer, should be given alternative, more aggressive or combination therapies to improve outcomes and survival of patients annually.

### Example 4

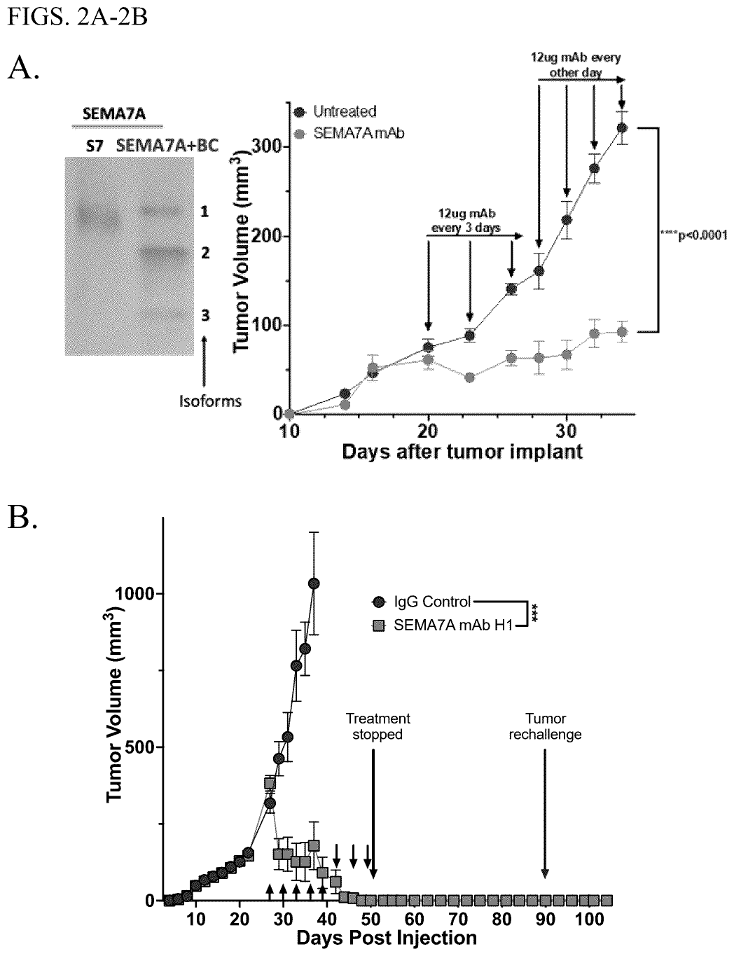

For initial in vivo studies with SmABHI. SEMA7A OE E0771 mouse mammary carcinoma cells were injected into mice. Twenty days after carcinoma cells were injected. SmAbH1 (e.g., 12 ug) was administered intraperitoneally to the mice every three days until day 36 post-carcinoma cell injection. Tissues were collected at day 36. Tumors, lungs, and LNs were harvested and processed for IHC and flow cytometry to determine the effect of SEMA7A mAb therapy on cells of the tumor microenvironment (TME). In an additional experiment, tumors were treated at day 28 post-injection with a novel SEMA7A directed antibody, monoclonal antibody clone H1. Fully regressed tumors were removed from treatment at day 50) and the animals were re-challenged with an additional tumor inoculation at day 90 to assess SEMA7A mAb effects. Finally, to examine changes in immune cells in the tumor microenvironment, tumors were treated at day 13 post-inoculation and harvested at day 18. During this time period, tumors were actively regressing (FIGS. 2A-2C).

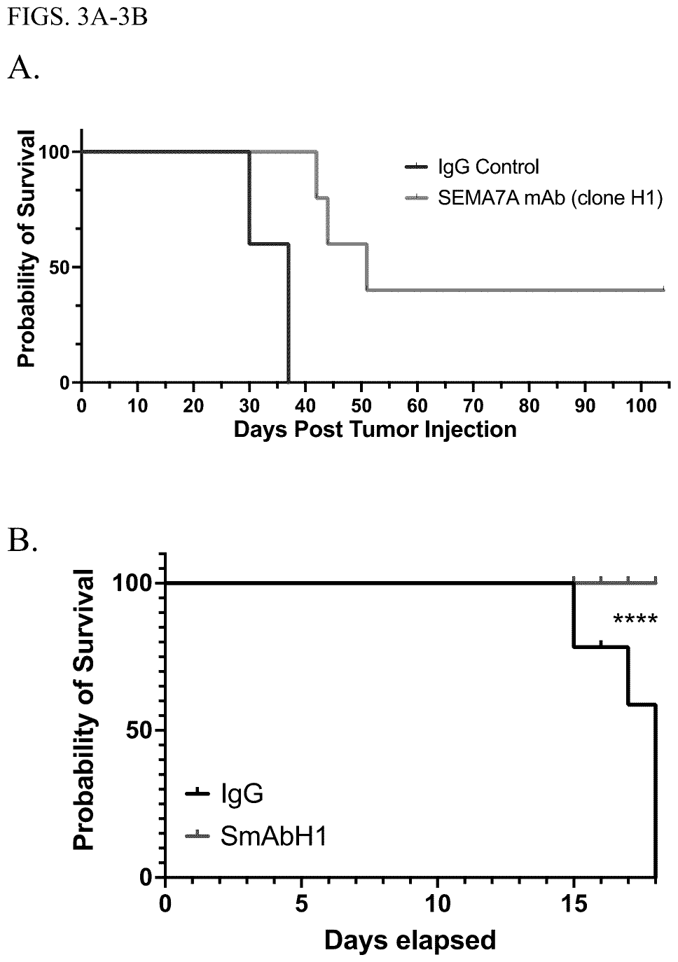

In these examples, in vivo studies indicated that treatment of SEMA7A OE tumors with SEMA7A mAbs markedly inhibited tumor growth (FIGS. 2A-2B). Additionally, mice treated with SEMA7A mAbs had a significant increase in survival time. Treated mice had double the survival time from about 35 days to about 75 days compared to untreated mice (FIGS. 3A-3B). This observation equates to an increase in probability of survival for humans of about 2.4 years to about 5.2 years using SEMA7A mAbs treatment alone.

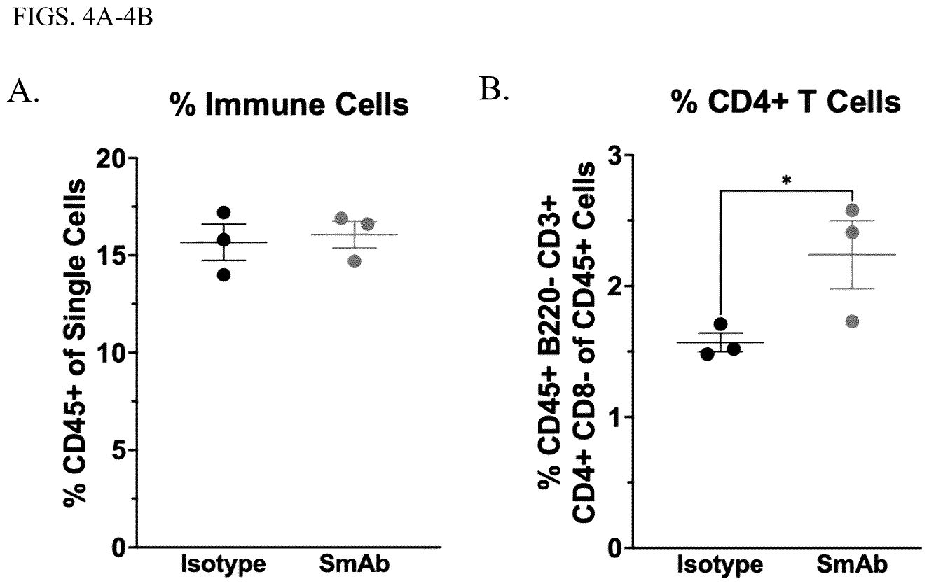

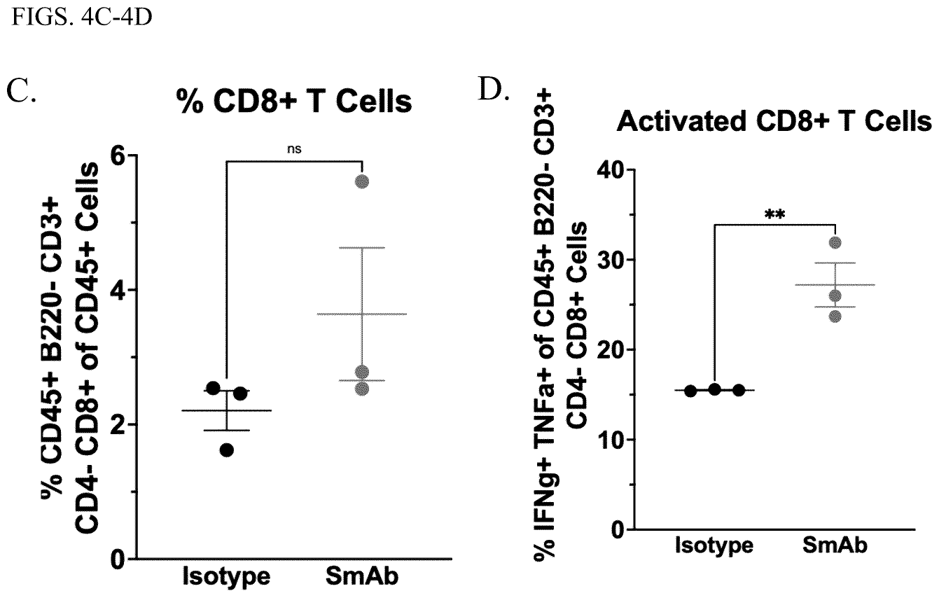

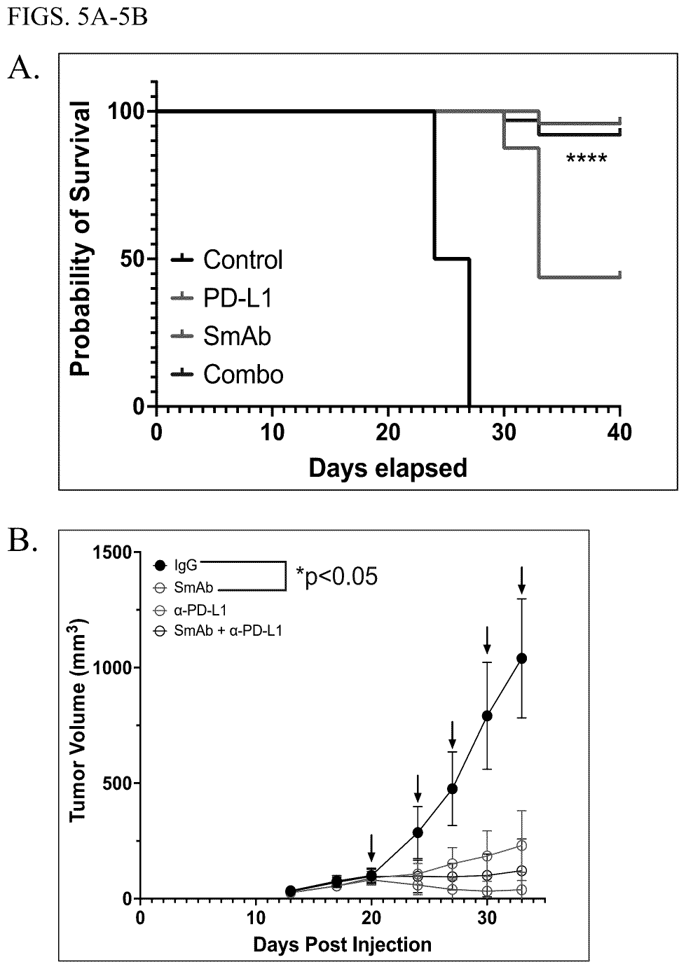

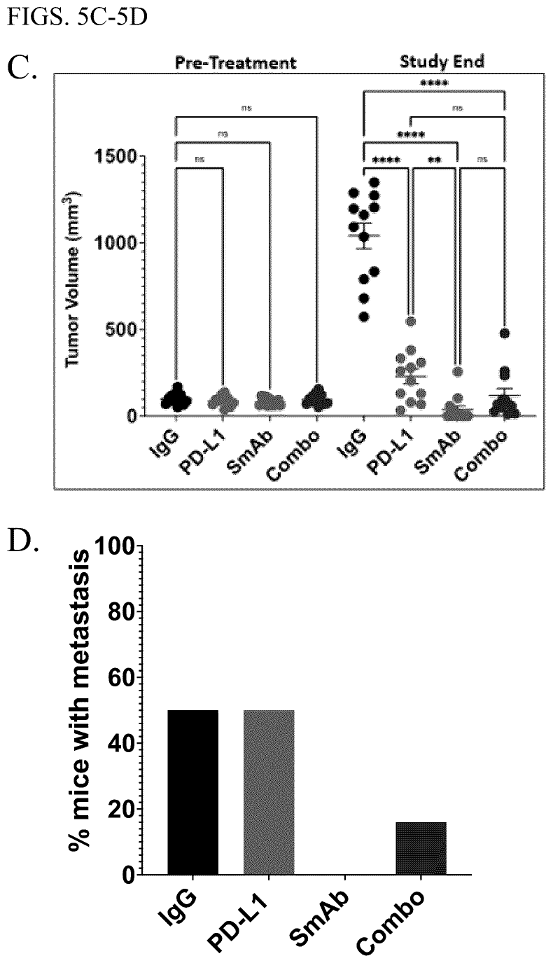

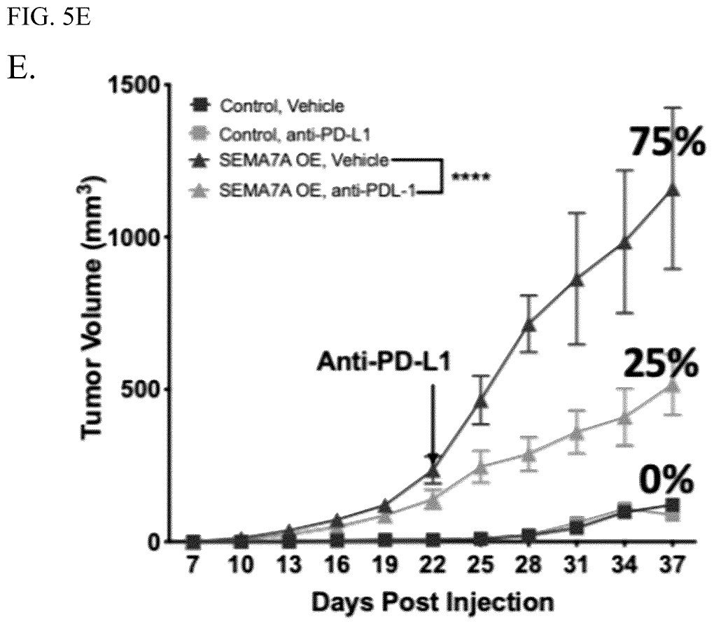

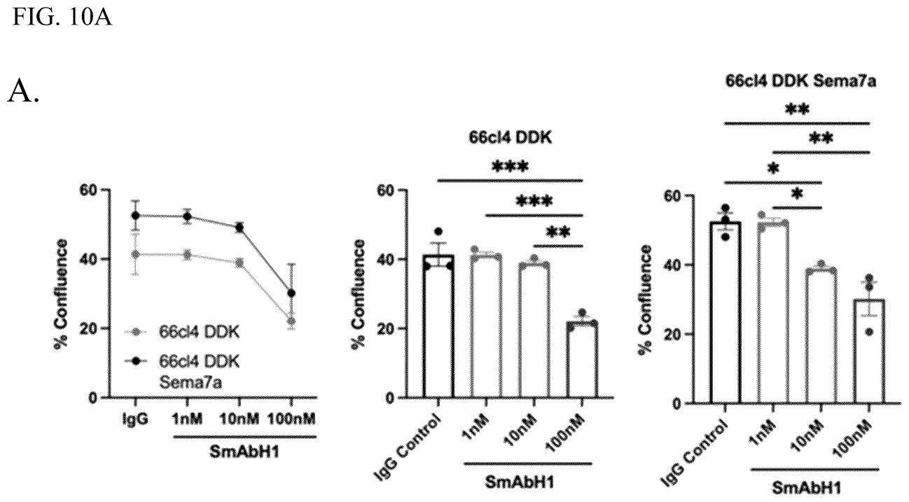

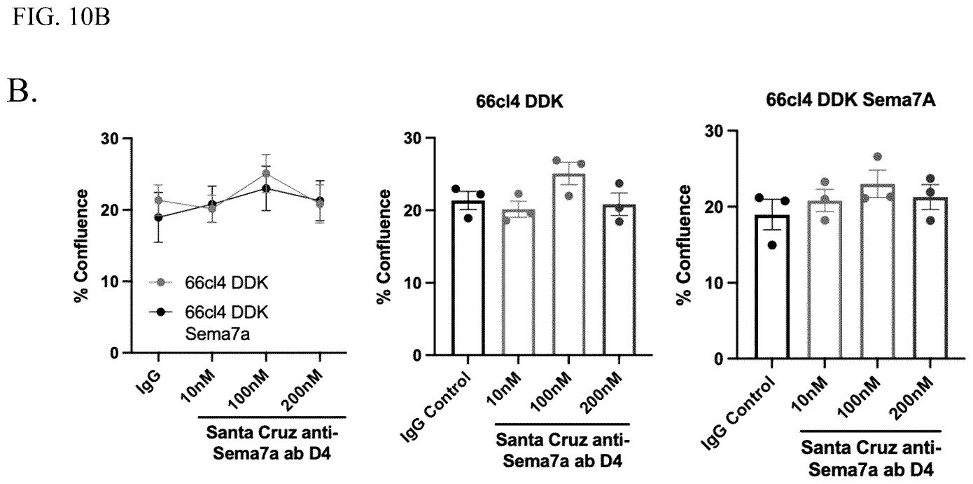

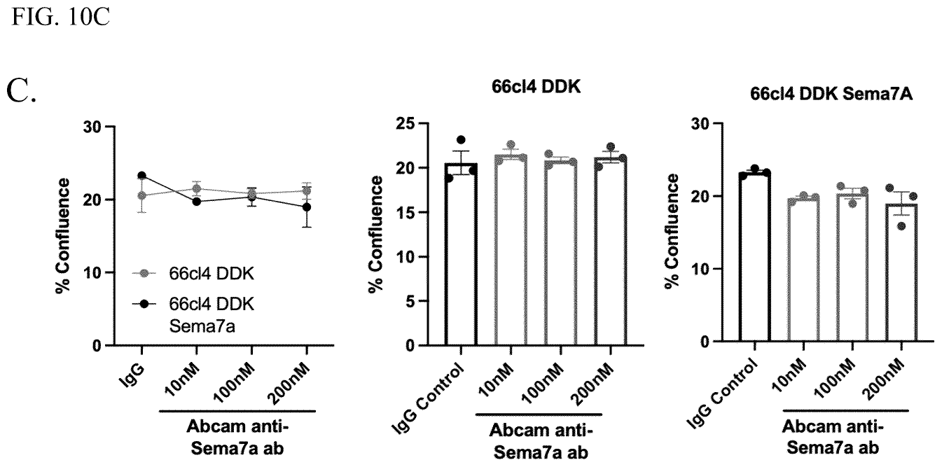

Flow cytometry analysis from in vivo studies indicated that treatment with SmAbH1 (SEMA7A monoclonal antibody) resulted in an increased presence of activated T cells in the tumors (FIGS. 4A-4D). Further. SmAbH1 was tested alone or in combination with a known immunotherapy (anti-PD-L1). These experiments demonstrated an increase in activated T cells in tumors exposed to SmAbH1 alone which was superior at reducing both tumor growth and metastasis when compared to anti-PD-L1 and the combination of both antibodies (FIGS. 5A-5E). Additionally. SEMA7A expressing tumors when exposed to anti-PD-L1 treatment alone did not fully regress, but metastasis was reduced (FIG. 10E).

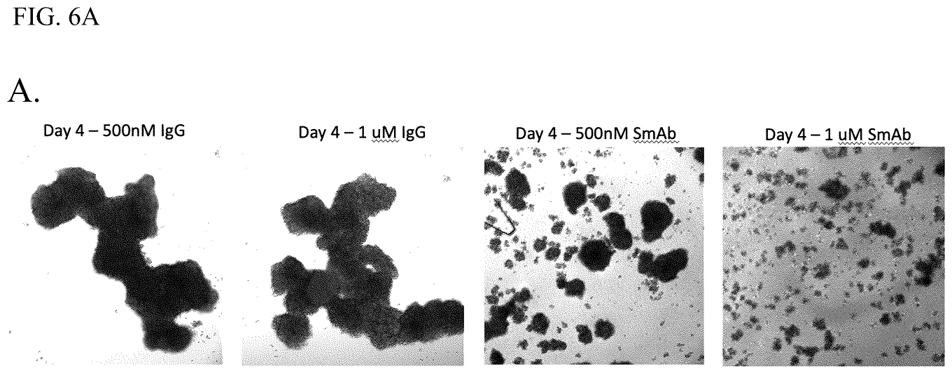

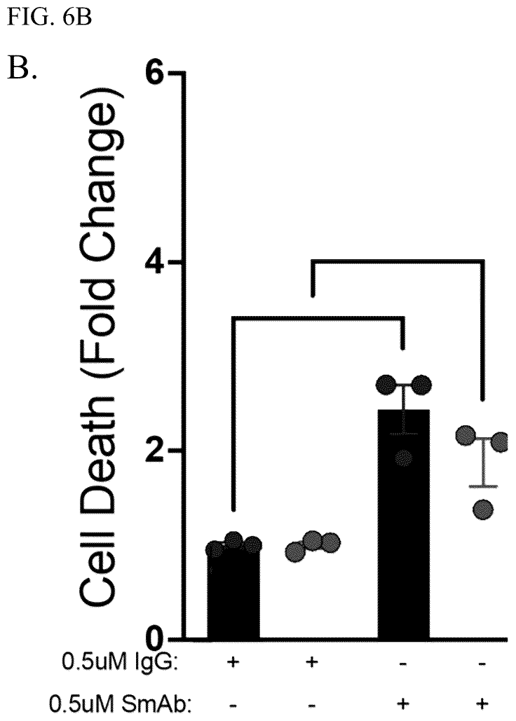

In another exemplary method, it was observed that SEMA7A was sufficient to promote stem cell phenotypes, including mammosphere formation, anchorage independent growth and resistance to chemotherapy, which is a property of cancer stem cells. Finally. SEMA7A expressing cells display increased functions and markers of cancer stem cells. However, treatment of cells with SmAbH1 was sufficient to reduce mammosphere formation and induce cell death (FIGS. 6A-6B).

These exemplary methods suggest that patients with SEMA7A expressing tumors can be resistant to current standard of care therapies and that alternative therapies, such as anti-PD-L1/PD-1 and use of an exemplary BCL-2 inhibitor: Venetoclax- or other therapies that target cell survival pathways, should be used alone or in combination with anti-SEMA7A antibodies or monoclonal antibodies to SEMAZA or epitopes targeted herein. Consistent with this, analysis of the cancer genome atlas (TCGA) reveals that patient tumors express high levels of SEMA7A, regardless of stage, and levels of SEMA7A are highest in younger patients and in patients with immune and stem cell enriched tumors. Finally, co-expression of SEMA7A and alpha-6 integrin, a partner for beta-1 integrin and stem cell marker, have decreased relapse free survival: this data supports a role for SEMA7A in promoting relapse in breast cancer patients regardless of current therapies.

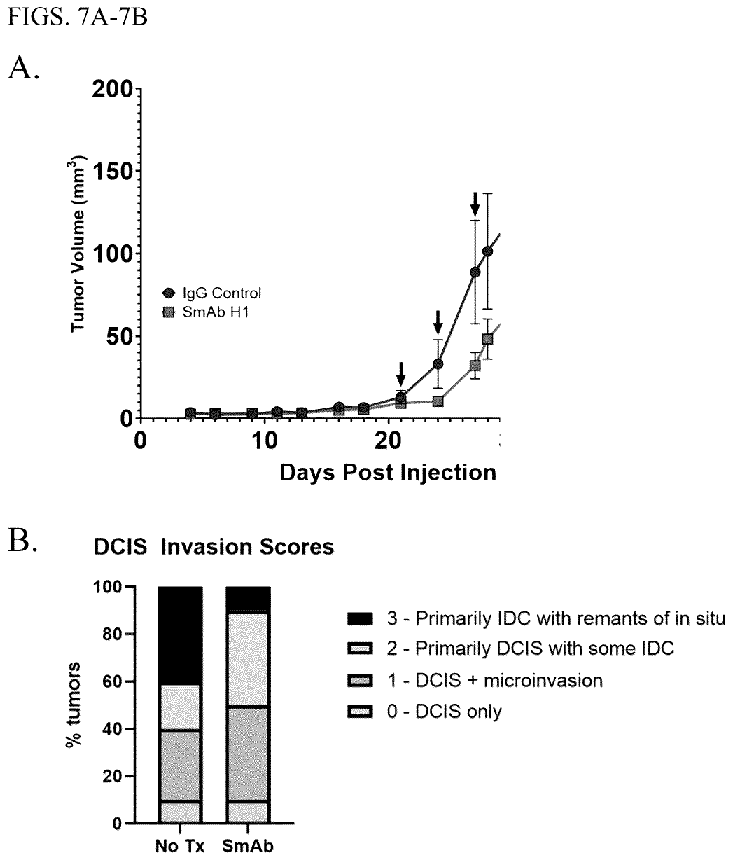

In another exemplary method, the ability of the SEMA7A monoclonal antibody (SmAbH1) to block or inhibit tumor growth and invasion of ductal carcinoma in situ (DCIS) lesions was demonstrated in a mouse model, which suggests that SEMA7A could be targeted at the earliest stages of breast cancer in order to reduce or prevent cancer progression, metastasis, and promote tumor cell killing (FIGS. 7A-7B).

### Example 5

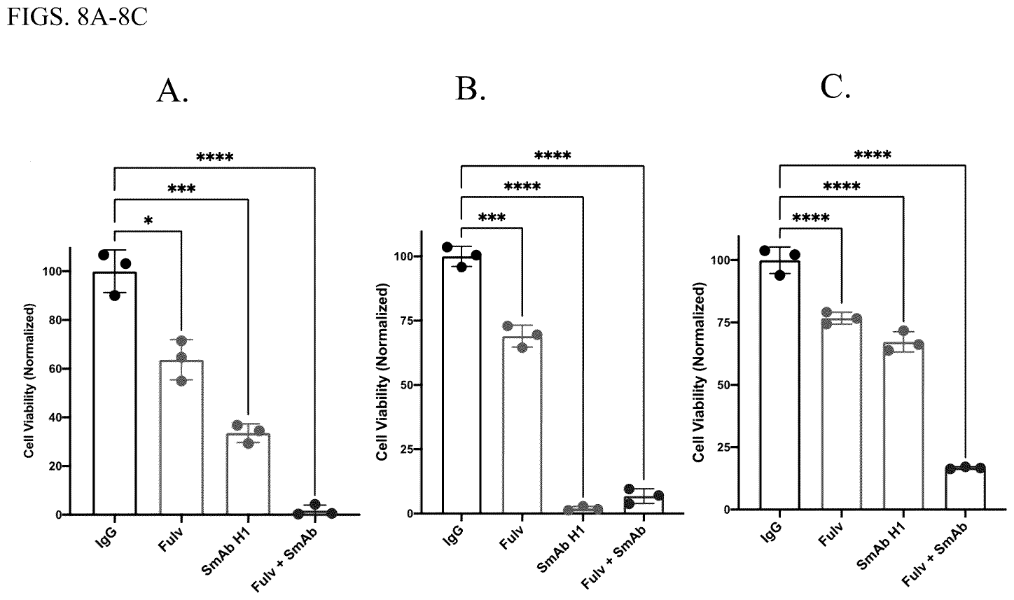

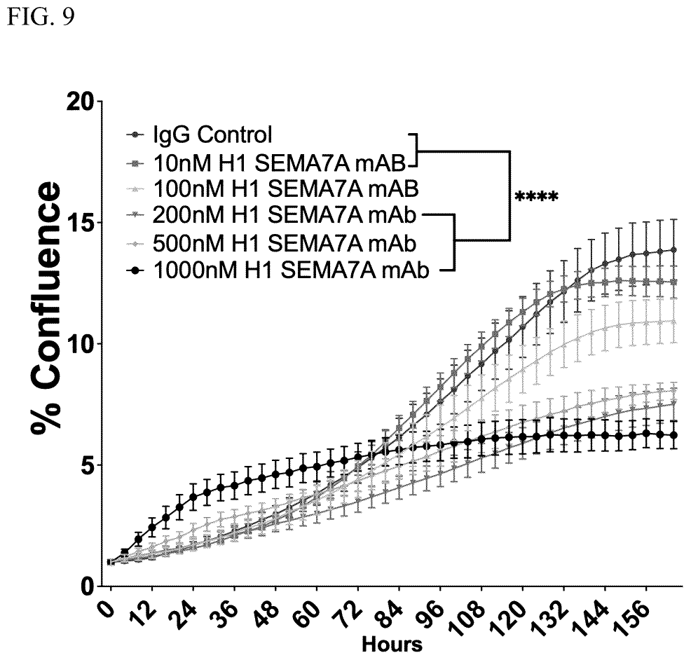

In another exemplary model, direct tumor cell killing by SEMA7A monoclonal antibodies (SmAbH1) was demonstrated in vitro. ER+SEMA7A expressing tumor cells (MCF7. TC11 and SSM2) viability was more significantly reduced by SEMA7A monoclonal antibody (SmAbH1) compared to anti-estrogen therapy (fulvestrant) (FIGS. 8A-8C). Additionally, in a model of ER-SEMA7A expressing tumors decreased viability was observed with SEMA7A monoclonal antibody (SmAbH1) in a concentration and time dependent manner, modeled in this example (FIG. 9). In another method, in the same model, the ability of the SEMA7A monoclonal antibody (SmAbH1) to decrease cellular confluence, a measure of cell viability, was observed which was greater than commercially available

SEMA7A directed antibodies, (e.g., SC-374432; Santa Cruz. Biotech and Ab23578: Abcam) (FIGS. 10A-10C). This data supports that the monoclonal antibodies to the epitope claimed herein can cure SEMA7A-related conditions and work better than the commercially available antibodies.

These exemplary methods suggest that SEMA7A monoclonal antibody (SmAbH1) could be utilized for treating both ER+ and ER-breast cancers alone or in combination with currently approved therapies.

In one exemplary method, data mining for combined molecular score in multiple types of cancer reveals that SEMA7A plays an important role for other types of cancer besides breast cancer. It was found that other cancers can benefit from reducing SEMA7A expression or translation such as endometrial, skin, liver, brain, ovarian, stomach, mesothelioma but the highest correlation was leukemia and pancreatic other than breast cancer (data not shown). It is noted that leukemia can be treated with the BCL-2 inhibitor (e.g., venetoclax) in addition to a SEMA7A monoclonal antibody against SEQ ID NO:2 or 3 or conjugate thereof. Given this observation, leukemia could also be treated with an anti-SEMA7A antibody disclosed herein alone or in combination with a BCL-2 inhibitor.

### Example 6

In another exemplary method, the ability of an anti-SEMA7A monoclonal antibody to bind to SEMATA was tested. In one exemplary method, CUH1 mAb was validated for binding to MCF7 SEMA7A over expressing cell lysate using Western Blot. Specifically. binding of the CUH1 mAb was detected on lysates from MCF7 control cells and SEMA7A overexpressing (OE) cells using 200 μg/mL of SmAbH1. MCF7 cells endogenously express SEMA7A (75 kDa) but OE cells were engineered to overexpress V5 tagged

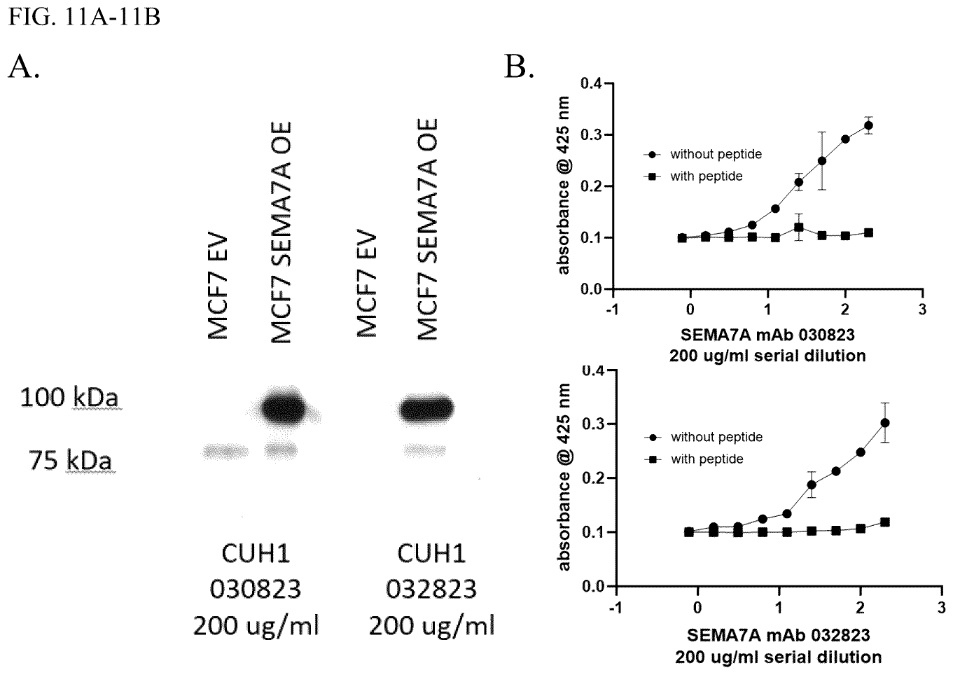

SEMA7A (100 kDa). As illustrated in FIG. 11A, the SmAbH1 antibody successfully bound to both endogenous (75 kDa) and overexpressed (100 kDa) SEMA7A. In another exemplary method. CUH1 mAb was validated for binding to purified SEMA7A protein using Enzyme-linked immunosorbent assay (ELISA). It was found that multiple lots of SmAbH1 bound to purified SEMA7A in a dose dependent manner (FIG. 11B).

### Example 7

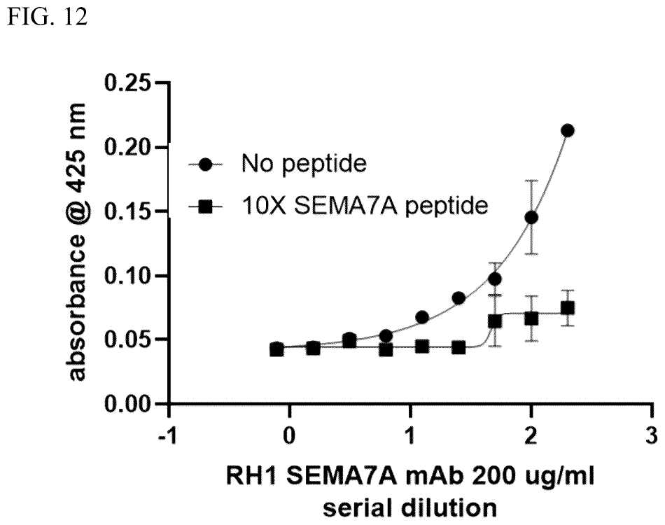

In another exemplary method, a recombinant anti-SEMA7A monoclonal antibody was produced and tested using an enzyme linked immunosorbent assay (ELISA) to evaluate its binding ability. In various exemplary methods, it was found that this recombinant antibody bound to purified SEMA7A in a dose dependent manner and inclusion of 10× immunizing peptide blocked this ability (FIG. 12).

### Example 8

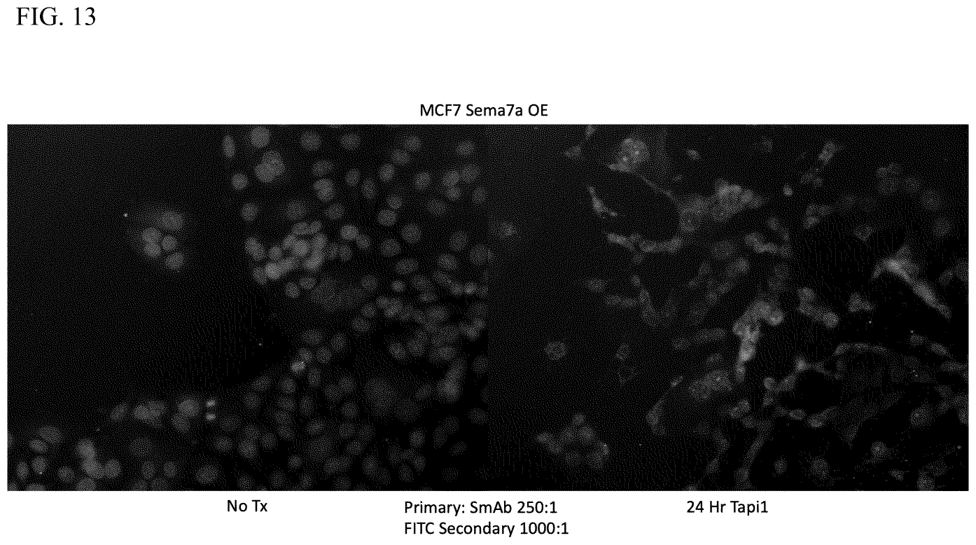

In another exemplary method, the ability of anti-SEMA7A monoclonal antibodies to bind and label cells was tested. In an exemplary method, human breast cancer MCF7A SEMA7A overexpressing cells were treated with TAPI-1, a TACE/ADAM17 inhibitor that blocks SEMA7A shedding from the cell surface, and then incubated with an anti-SEMA7A monoclonal antibody (250:1) before visualization with a FITC secondary antibody (1000:1). As illustrated in FIG. 13, the anti-SEMA7A monoclonal antibody labeled the cell lines treated with TAPI-I.

### Example 9

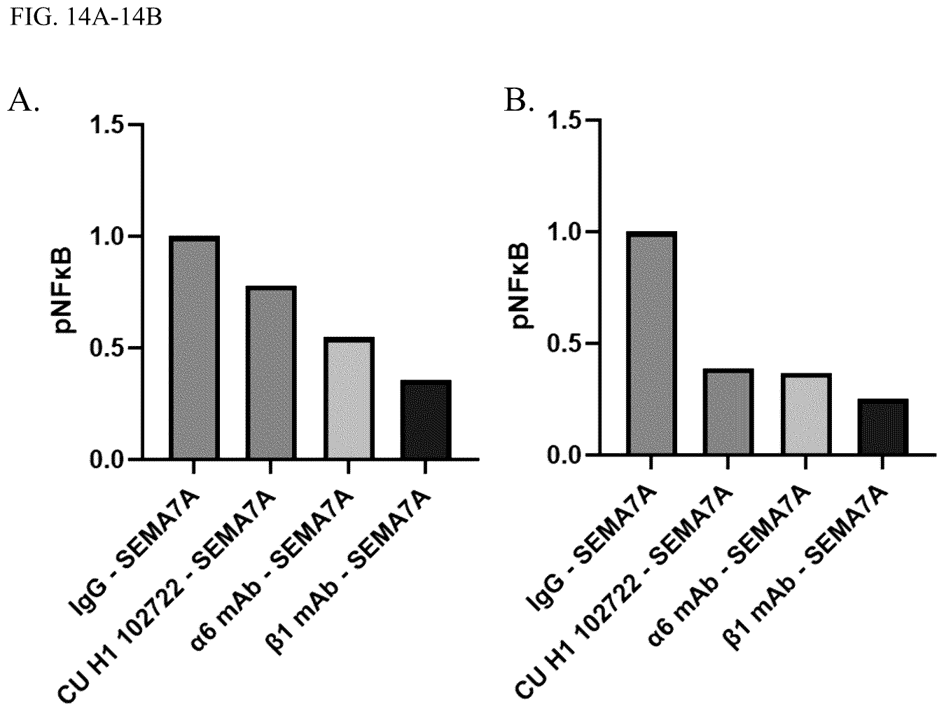

In another exemplary method, the ability of the anti-SEMA7A monoclonal antibodies to affect cellular processes related to cell survival was tested. In one exemplary method, human triple negative breast cancer cell lines with low (MDA468) or high (MDA231) levels of SEMA7A were treated with SmAbH1 (CUH1) or commercially available monoclonal antibody-based inhibitors of an integrin receptor for SEMA7A (α6β1) and then evaluated using Western blot for phosphorylation of NFκB (pNFκB), a key survival marker. It was found that SmAbH1 (CUH1) blocked downstream NFκB activation (as measured by pNFκB levels) in both cell lines but the effect was stronger in a cell line with high SEMA7A expression (MDA231) (FIGS. 14A-14B). Notably, the effect of SmAbH1 on downstream NFκB mirrored integrin blockade (e.g., by antibody-based inhibitor of α6β1) in cells with high SEMA7A expression.

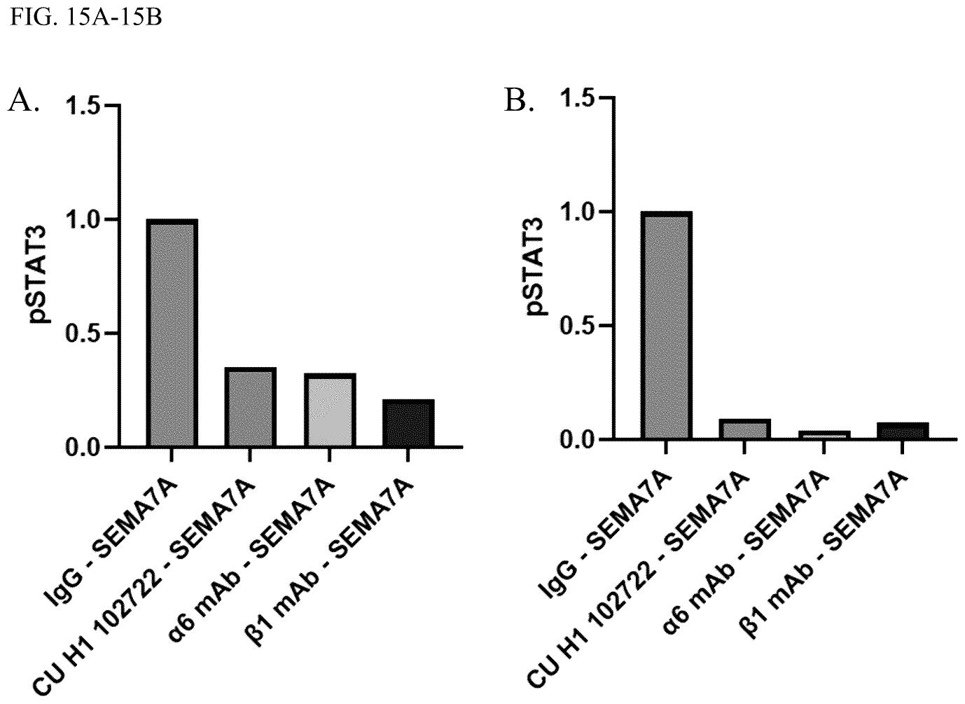

In another exemplary method the human triple negative breast cancer cell lines with low (MDA468) or high (MDA231) levels of SEMATA were again treated with SmAbH1 (CUH1) or commercially available monoclonal antibody-based inhibitors of an integrin receptor for SEMA7A (α6β1) and then evaluated using Western blot for phosphorylation of STAT3 (pSTAT3). In these exemplary methods, it was demonstrated that the SmAbH1 (CUH1) antibody was capable of blocking block downstream STAT3 activation similar to integrin blockade in both cell lines (low and high SEMA7A expression) (FIGS. 15A-15B).

These exemplary methods demonstrate that anti-SEMA7A antibody treatment can directly affect downstream pathways controlled by SEMATA and further suggest that anti-SEMA7A antibody treatment can directly impact cell survival in any cell population that expresses SEMA7A.

### Example 10

In one exemplary method, in vivo treatment of a human subject with a composition including SmAbH1 (CUH1) will be assessed. A cohort of patients that have been diagnosed with breast cancer, with a subset that have been further diagnosed with postnatal breast cancer (PPBC), are selected. In various exemplary methods, the cohort will be divided into at least two testing groups which will each receive either a standard therapy, a placebo antibody plus standard therapy, or a standard therapy plus SmAbH1. Doses of the composition including SmAbH1 will be administered by a pre-determined regimen (e.g., every other day, weekly or every other week) will be administered in for example, a single bolus injection. Clinical outcomes will be evaluated for each group and will include measures of tumor size, metastasis, or recurrence before, during and after treatment. Patients with treated and untreated PPBC will be compared to those with other types of breast cancer to evaluate success of SmAbH1 treatment to reduce or eliminate the cancer in the patient. Measurements of SEMATA expression in patient tumor samples will also be obtained. It is predicted that, in certain exemplary methods. SmAbH1 treatment will have a stronger effect (e.g., as measured by decreased tumor size, decreased metastasis, or decreased recurrence) in patients with higher SEMA7A expression as compared to patients with lower SEMA7A expression.

### Example 11

In another exemplary method, in vivo treatment of other conditions characterized by SEMA7A dysregulation, expression, or overexpression in human subjects using a composition of SmAbH1 (CUH1) to detect SEMA7A in one or more sample from the subjects will be assessed. Patients having cardiac conditions (e.g., myocardial infarction). inflammatory conditions such as arthritis, colitis, airway inflammation, and Guacher disease. lung or liver fibrosis, or multiple sclerosis, or any other health condition where SEMA7A is expressed or over-expressed and/or contributes to adverse health conditions in a subject will be screened to assess levels of SEMA7A expression in patient tissues, using standard methods In some exemplary methods, patient samples will be probed with an SmAbH1 antibody to assess levels of SEMA7A expression. Patients having a predetermined threshold level of SEMA7A expression will be administered SmAbH1 antibody or a control vector and therapeutic outcomes evaluated. It is predicted that in various exemplary methods. SmAbH1 treatment will result in an improved therapeutic outcome (e.g., increased survival, reduction in symptoms, decreased pain, etc.) in the patient cohort and that the levels of SEMA7A expression in the patient's tissues will correlate with beneficial or improved therapeutic outcome.

### Materials and Methods

### Immunohistochemistry

Tissues were formalin fixed and paraffin embedded as previously described. Four μm sections of paraffin-embedded human tissue were deparaffinized and pretreated with 1× Dako Target Retrieval solution under pressure for 5 minutes. Slides were prepared in a Dako Autostainer using Semaphorin 7a primary antibody (SEMA7A C-6, Santa Cruz). Immunoreactivity was detected using Envision+ Mouse secondary antibody (Dako). 3,3′-diaminobenzidine was used as the chromogen (Dako, 10 min). Hematoxylin was used as the counterstain (Dako, 6 min). The majority (>80%) of cases had Ki-67% index staining centrally performed as previously described.

Staining quantification was done using Aperio ImageScope software (Leica Biosystems). Histological sections were digitally scanned using Aperio ScanScope3 equipment. Each section was assessed for normal-adjacent tissue and invasive breast cancer (IBC), and peritumoral lymphatic vessel density in a blinded manner. Tissues were subsequently annotated for representative regions of each tissue category present. Annotated regions were analyzed for percent positive staining using a proprietary color deconvolution algorithm created in ImageScope. Percent positive was calculated as the sum of total medium and strong (M+S) positive signal, divided by the total annotation area, and multiplied by 100.

### Statistical Analysis

Two-sample independent t-tests were used to compare the distributions of the continuous outcomes between Nullip vs PPBC groups, and the paired comparisons between subgroups: Fisher's exact tests were conducted to compare the distributions of the categorical variables between Nullip vs PPBC groups, and the paired comparisons between subgroups. For all tables and dot plot graphs, continuous outcomes are expressed as the mean+/−SEM. and categorical outcomes are presented as frequencies and the percentages. For Kaplan Meier analysis, Recurrence Free Survival (RFS) is defined as the time from the date of diagnosis to the date of local recurrence, reginal recurrence, or the last date of follow-up, whichever comes first. The non-parametric log rank test was conducted to compare the recurrence free survival curves of SEMA7A low vs high groups for PPBC patients and for Nulliparous patients separately. The optimal cutoff level of SEMA7A were obtained using R package “survminer′20. The cutoff level of SEMATA is set to 36.1, which is slightly above the average observed in the PPBC group and can describe the bimodal feature of the distribution of the SEMA7A in this group reasonably well and is also biologically and clinically plausible. P-values less than 0.05 were deemed statistically significant. The datasets generated during and/or analyzed during the current study are available from the corresponding author on reasonable request.

### SEMA7A ELISA

Human SEMA7A ELISA (Biomatik, Cat #EKU07269) was used according to manufacturer's instructions. Breast cancer patient serum samples were obtained from our collaboration with Dr. Virginia Borges and collected under an IRB-approved protocol. Samples were selected based on tumor level expression of SEMATA and sample availability. Generating SEMA7A-Antibody Drug Conjugate

Fab-ZAP Mouse antibody drug conjugation kit (KIT-48) was obtained from Advanced Targeting Systems (San Diego, CA), used with human anti-mouse-SEMA7A antibody (sc-374432, clone C6, lot #K1512 SantaCruz Biotechnologies, Dallas TX) according to the manufacturer's instructions, with modifications discussed in results section.

### Cell Culture and Reagents

Overexpression plasmid (SEMA7A-Fc) was obtained. Control plasmid (pcDNA3.1) was obtained from H. Ford (CU Anschutz Medical Campus, Denver, CO). All other overexpression plasmids (p304-V5-Blasticidin and V5-SEMA7A) were obtained from the Functional Genomics Core at the CU Anschutz Medical Campus and overexpression was confirmed via qPCR and Western blot analysis.

Monoclonal antibody generation

Mice (5) were injected with KLH-conjugated SEMA7A Peptide. Serum was then collected for testing. ELISA, Western Blot and IHC using serum to detect purified SEMA7A and peptide. Mice were then boosted with peptide and testing serum until detection is at a desired level. Spleens were then harvested and B cells from spleens were fused with myeloma cells to create hybridomas. Positive hybridomas were then selected and monoclonal antibody can be harvested from the supernatant.

### Proliferation Assays

In vitro growth, proliferation rate, adhesion, and motility assays

For proliferation assays, cells were plated at 2000 cells per well of a 96-well dish. Four pictures of different regions in each well were taken every four hours for 48 h using an IncuCyte ZOOM (Essen BioScience, Ann Arbor, MI, USA). Percent confluence was determined using the IncuCyte ZOOM software (Essen BioScience). 66cl4 Sema7A DDK cells were plated in a 96-well plate in triplicate at 1,500 cells/well. They sat down over 24 hours and then were treated with 1000 nM IgG control, 10 nM SmAbH1, 100 nM SmAbH1, 200 nM SmAbH1, 500 nM SmAbH1, or 1000 nM SmAbH1. The plate was promptly transferred to the IncuCyte where photos were taken and analyzed every 4 hours for 7 days. For additional treatment studies in MCF7, TC11 and SSM2 cells (10,00/well) were plated in a 24-well plate in complete media and allowed to sit down over 24 hours. At 24 hours after plating, cells were treated with 500 nM IgG control, 500 nM SmAbH1, 8 nM Fulvestrant or the combination. The plate was incubated at 37 C for 48 hours, then fixed with 10% NBF and stained with crystal violet.

### Trans-well Invasion Assays

For transwell invasion assays, 25,000 cells per well were plated in triplicate precoated transwell inserts with 1% horse serum as chemoattractant and quantified by Image J.

### In Vivo Assays

E0771 model: Female C57/BL6 mice were injected orthotopically into the left and right #4 mammary fat pads with 200,000 E0771 SEMA7A OE mouse mammary carcinoma cells. Tumors were measured every day once tumors became palpable (3 days post injection). When the average tumor volume for the full cohort (all 10 mice) reached 150 mm3, mice began treatment with either SmAb H1 or IgG2 isotype control. Treatment was administered every other day by intraperitoneal injection at a concentration of 12 μg. 50 μg or 250 μg (F50 μg in 100 μl total volume (final concentration=2 μg/μl). For PD-L1 studies PD-L1 was co-delivered at 250 μg.

DCIS model: Female SHO mice, aged 6-8 weeks old, are injected with 250,000 MCF10ADCIS GFP human breast cancer cells into both #4 mammary fat pads, then palpated and measured three times a week with digital calipers and tumor visualization is aided by use of the illumatool light source with GFP filter so we can accurately measure tumors. Treatment with SmAb started at day 21 post injection. SmAb treatment is given by IP injection every third day. Weights are taken every treatment day and mice are monitored for weight loss and other health/behavior changes.

### Flow Cytometry

Tumors were separated from the mammary gland and placed in six-well plates with 2 mL of Click's media without mercaptoethanol or L-glutamine (Irvine Scientific, Santa Ana, CA), where they were minced with scalpels, digested with 500 units/ml collagenase type II and IV and 20 μg/ml DNase (Worthington Biochemical Corporation, Lakewood, NJ) and incubated for 1 h at 37° C. The tissue suspension was then filtered through a 100 μm strainer and washed with Click's. The filtered cells were centrifuged at 1,400 RPM for 5 min, the supernatant was removed, and the pellet was resuspended in 1 mL FACS buffer (500 mL 1× HBSS pH 7.4, 0.1% BSA, 0.02% sodium azide, up to IL ddH2O). The tumor cells were stained with BD viability 510 dye prior to staining with CD45 (clone30-F11), CD8a APC/Cy7 (clone 53-6.7) (1:400), CD4 APC or PerCp-Cy5.5 (clone RM4-5) (1:300), CD8 T cells were identified from live, CD3+/CD8+Cells were run on the DakoCytomation CyAn ADP flow cytometer (Fort Collins, CO) or FACs Canto II, acquired using Summit software or Diva Software, and analyzed with FlowJo software (Tree Star, Ashland, OR). Geometric mean fluorescence intensity (gMFI) was calculated with FlowJo software. Cells were isolated from the tissue and treated with or without (unstimulated controls) phorbol 12-myristate 13-acetate (PMA) (20 ng/ml) (Sigma. St. Louis, MO) plus ionomycin (1 ug/ml) (Sigma, St. Louis,MO) for 4-6 h at 37 degrees in the presence of 2 μg/ml of brefeldin A (Adipogen, San Diego, CO) in RPMI+2.5% FBS. Cells were then stained with CD8, CD45, CD4, Gating was determined based on unstimulated controls. All antibodies were purchased from Biolegend (San Diego, CA).

### Data Mining Assays

Kaplan-Meir plotter was used to assess relapse-free survival (RFS) data for 5,143 breast cancer patients with a mean follow-up of 69 months. SEMA7A was queried and stratified into high and low expression groups using the autoselect best cutoff level function. The generated data were exported, graphed, and analyzed in GraphPad Prism to calculate hazard ratio (HR) with 95% confidence intervals and log-rank P values. The Gene Expression-Based Outcome for Breast Cancer Online platform (http://co.bmc.lu.se/gobo/) was used to query SEMA7A expression in 1,881 available sample tumors and a panel of 51 breast cancer cell lines. Data were stratified as high and low SEMA7A based on median expression and reported by molecular subtype. Outcome data were reported only in patients with ER+breast cancer. The Oncomine Platform was used to query SEMA7A in available breast cancer data sets. The P value threshold was set to 0.05. All fold changes and gene ranks were accepted. Data are reported from the molecular subtype analysis, correlation with stage and grade, and drug-sensitivity screens.

### Cell Culture

MCF10A/MCF12A cells were obtained and cultured, according to ATCC. MCF10DCIS.com cells were obtained. Cells were validated by the DNA sequencing and found to be pure populations of their respective cell lines. Cells were regularly tested for mycoplasma throughout the studies. Cells were sub-cultured as previously described (reference), or according to ATCC standards. Exogenous SEMA7A protein utilized in our studies was purified from media of cells stably transfected with an Fc-tagged version of SEMA7A by published methods. Control plasmid (pcDNA3.1) was obtained.

shRNA Knockdown and Overexpression

shRNA plasmids targeting Sema7a, and a negative control shRNA (SABiosciences), were amplified in E, coli and plasmid DNA was isolated by Plasmid Maxi-Prep (Qiagen). MCF10DCIS.com-GFP cells were cultured overnight to ˜80% confluence. 1 μg/μL of each shRNA was added to Transfectagro Reduced-Serum Medium (Corning) and incubated for 15 min with 4 μL of X-treme Gene HP DNA transfection reagent (Roche). Transfected cells were selected for hygromycin resistance. Stable knockdown was confirmed by qPCR. Negative control cells were transfected with a scrambled artificial sequence not matching human, mouse, or rat. Overexpression plasmid (SEMA7A-Fc) was a generous gift from R. Medzhitov (Yale University, New Haven, CT). All other over-expression plasmids (p304-V5-Blasticidin and V5-SEMA7A) were obtained from the Functional Genomics Core at the CU Anschutz Medical Campus and overexpression was confirmed via qPCR and western blot analysis. KD and overexpression were determined using established methods.

### Animal Studies

All animal procedures were approved by the University of Colorado Anschutz Medical Campus Institutional Animal Care and Use Committee. Sema7atm 1 Alk/J mice (a generous gift from Alex Kolodkin at John's Hopkins University) and C57Bl/6 (Jax) were housed and bred as using standard methods. Briefly, C57BI/6 SEMA7Atml Alk females were bred with WT C57Bl/6 males to induce pregnancy, lactation, and postpartum involution. Pup numbers were normalized to 6-8 per dam after birth to ensure for adequate lactation. Postpartum involution was initiated by forced weaning of pups at the peak of lactation (L10-L12). #4 right and left mammary glands were harvested from age-matched nulliparous animals and postpartum dams at involution days 1,2,4,6,8,10,14, and 28. For flow cytometry, inguinal lymph nodes were removed prior to tissue digestion. #3 mammary glands were also harvested, formalin-fixed, paraffin-embedded, and 5 μm sections generated for histologic/immunohistochemical analysis.

### In Vitro Survival Assays

MCF10A and MCF12A cells were cultured with 200 ng/ml purified SEMA7A or PBS vehicle control in adherent (tissue culture plastic) or forced suspension conditions (μltra-low attachment plates (Corning, Corning, NY, USA: #3473)). Cells were seeded at a density of 1000 cells/well in a 96-well plate plus media only wells as controls. Cell death was measured 24 h post seeding via luminescence using the Caspase Glo assay (Promega, Madison, WI, USA: #G8090). Annexin-V/7AAD staining (Biolegend, San Diego, CA, USA: #640930) and pAKTS473 (ThermoFisher: Waltham, MA, USA, #17-9715-42) were also used to confirm cell viability by flow cytometry. See flow cytometry methods for staining protocol. Function blocking antibodies for bl (CD29-9EG7: BD Biosciences: #550531) and a6 integrin (ThermoFisher: #14-0495-82) were used to disrupt integrin signaling in the presence of SEMAZA or as antibody alone controls to determine off-target effects. IgG controls were also used. 9EG7 was used at a concentration of 0.6 μg/ml while GoH3 was used at a concentration of 100 μg/mL for inhibition studies. Cells were cultured with anti-integrin inhibitors at time of seeding.

### Label Retention Stem Cell Assay

MCF10A and MCF12A cells were labeled with CellTrace Violet (ThermoFisher: #C34557) for 20 min at 37° C., according to the manufacturer's instructions. A sample of labeled cells was analyzed by flow cytometry to determine labeling efficiency and fluorescent intensity at time of seeding. Single cells were seeded at a density of 4000 cells/well in μltra-low attachment 24-well plates to induce mammosphere formation. Cells were cultured in normal culture media for 7 days and were subsequently counted, dissociated enzymatically in Accutase, (Stem Cell Technologies, Vancouver, Canada: #07922), and analyzed by flow cytometry. See flow cytometry methods for detailed information.

### Paclitaxel Resistance Studies

MCF10DCIS.com cells were treated with 100 nM paclitaxel (Millipore Sigma, Burling, MA, USA #T7402-5 MG) for 24 h prior to analysis by flow cytometry. Dose was determined experimentally based on cell viability and cancer stem cell enrichment. Drug resistance was measured by label-retention and mammosphere formation as described above and % CD44+ (Biolegend: #103018) CD24-(Biolegend: #311104) of singlets was used as a molecular definition of drug-resistant stem cells. See label retention stem cell assay and flow cytometry methods for additional details.

### Additional Flow Cytometry

Single cell suspensions were generated from cultured cells via enzymatic harvest with Accutase. Mouse mammary tissues were minced with scalpels, digested in Click's media containing 500 units/mL Collagenase II (Worthington, Columbus, OH, USA: LS004174) and Collagenase IV (Worthington, LS004186) and 20 μg/mL DNAse (Worthington, LS002004) for 1 h at 37° C., with occasional trituration and strained through 70 um filters to generate single cell suspensions. Single cells were stained at 4° C., for 30 min and washed with PBS+2% FBS. Cells were filtered through 30 μm filters, diluted to <106 cells/ml, and analyzed with the ZE5-YETI flow cytometer. Fixation/Permeabilization kit (BD Biosciences, Franklin Lakes, NJ, USA: #554715) was used for intracellular antigen staining. Data were analyzed with Kaluza software. Single stain, FMO, and unstained controls were used to set gates. Single cells were lineage depleted using CD45-(BD Biosciences: #564279)/CD31-(BD Biosciences: #745436) gating.

### Immunohistochemistry

Tissues were formalin-fixed and paraffin-embedded. Hematoxylin and Eosin staining was used to define morphological features. Antigen retrieval was performed using target retrieval solution (Dako, Glostrup, Denmark: #S1699) for CC3 (Cell Signaling Technologies, Danvers, MA, USA: #9661) and Perilipin (Cell Signaling: #3470) or (EDTA Dako cat #S2367) for pSTAT3 (Cell Signaling Technologies: #9145). ImmPRESS polymer anti-rabbit IgG secondary reagent (Vector Labs, San Francisco, CA, USA t #MP-7401-15) was used for secondary staining of CC3 and pSTAT3 stained tissues and anti-rabbit secondary (Dako #K4003) for perilipin stains. DAB (Vector Labs: #SK-4105) and counterstaining were performed with hematoxylin (Vector Labs #H-3401). Alveolar area was measured on H&E-stained tissues in ImageJ on 10 alveoli/field: 5 fields/tissue. 10 fields/tissue were analyzed for positive CC3, pSTAT3, and perilipin stained tissues using CellSense Dimension software count and measure feature on regions of interest.

### Data Mining

Analysis of SEMA7A in breast cancer versus normal was performed on http://ualcan.path.uab.edu/index.html by selecting breast cancer samples from The Cancer Genome Atlas (TCGA). Five-year relapse-free survival (RFS) curves for Sema7a (Affy ID: 230345_at) and Itga6 (Affy ID: 201656_at) were generated in KM plot using default parameters and no restrictions. Patients split by median survival. For multi-gene analyses, genes were filtered by median expression.

### Statistical Analysis

Unpaired t-test and one-way ANOVA were performed in GraphPad Prism. Nonparametric equivalent analyses were used for samples with uneven distribution or unequal standard deviations, p values of <0.05 were considered significant. Error bars represent mean+standard error of the mean. All in vivo studies were replicated in at least three mice and in vitro studies performed in technical and biological triplicate with representative data presented.

### Enzyme-Linked Immunosorbent Assay (ELISA)

The MultiSorp ELISA 96 well ELISA plate (Thermo Scientific) was coated with 100 μl per well of 50 μg/ml of purified SEMA7A in Coating Buffer A (Thermo Scientific CB07100) and allowed to attach overnight with gentle rocking at 4 degrees. For the peptide competition, half of the antibody was incubated in an Eppendorf tube overnight at a ratio of 1:10 with the SEMA7A peptide on a rotator at 4 degrees. The plate was washed with 200 μl per well of 1× wash buffer (Thermo Scientific WB01) and blocked with 200 μl 1× assay buffer (Thermo Scientific DS98200) for 2 hours and room temp with gentle rocking. 1× assay buffer was removed and a 200 μl serial dilution of SMEA7A mAb in 1× assay buffer was made from columns 1 through 9, with column 10-12 getting 1× assay buffer only. The plate was incubated overnight with gentle rocking at 4 degrees. The plate was washed twice with 200 μl of 1× wash buffer. 100 μl of 200 ng/ml Goat Anti-Mouse IgG HRP (Abcam 97040) in 1× assay buffer was added to columns 1-11, with column 12 getting 1× assay buffer only. The plate was incubated for 2 hours with gentle rocking at room temp. The plate was washed four times with 200 μl 1× wash buffer. 150 μl SuperSignal Femto for ELISA (Thermo Scientific) was mixed 1:1 added to each well and mixed for 1 min and then left to rock at room temperature for 30 minutes. The absorbance was read at 425 nm on a microplate reader.

### Western Blot

MCF7 cells were lysed with RIPA containing EDTA (Thermo Scientific) and spun at 14K for 15 min. The supernatant was then quantitated using a Qubit Assay (Invitrogen) standard protocol and mixed 3:1 with Laemmli Sample Buffer (BioRad) with 2-mercaptoethanol and heated at 70 degrees for 10 min. 100 μg of lysate was loaded onto 4-20% Tris-Glycine gel (BioRad) and transferred to a PVDF membrane (Milipore). The membrane was blocked with 5% Bovine Serum Albumin (BSA) for 3 hours and then probed with 200 μg/ml of CUH1 in 5% BSA overnight at 4 degrees with gentle rocking. The membrane was washed three times 15 minutes with TBS-T and then probed with 10 ng/ml Goat Anti-Mouse IgG HRP (Abcam 97040) in 5% BSA for 1 hour at room temp. The membrane was washed and then incubated with 1:1 mix SuperSignal West Pico (Thermo Scientific) for 4 min at room temp.

### Immunofluorescence

Cells were fixed with 2% PFA or 1:1 acetone-methanol for 30 minutes at 4C, before washing once with PBS and blocking with 1% BSA in PBST for 1 hour at room temperature. After removal of the blocking solution, cells were incubated in primary antibody in 1% BSA in PBST overnight at 4° C. The primary antibody (SmAbH1 (lot 022823) was prepared at a 1:250 concentration by adding 4 μL stock solution to ImL 1% BSA in PBS. After primary removal cells were washed with PBS for 10 min×3 and incubated in secondary antibody in 1% BSA in PBS for 1-2 hours at room temperature (covered from light). The secondary antibody (Donkey anti-Mouse IgG Alexa Fluor 488 (Invitrogen A-21202)) was prepared at 1:1000 concentration by adding 1 μL into ImL 1% BSA in PBS. After removal of secondary antibody cells were washed with PBS for 10 min×3. VECTASHIELD Antifade Mounting Medium with DAPI (Vector Labs H-1200-10) was added and the cells were sealed with a glass coverslip. 20× images were obtained of the stained cells using the DAPI and FITC channels.

The foregoing discussion of the disclosure has been presented for purposes of illustration and description. The foregoing is not intended to limit the disclosure to the form or forms disclosed herein. Although the description of the disclosure has included description of one or more embodiments and certain variations and modifications, other variations and modifications are within the scope of the disclosure, e.g., as can be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

1. A monoclonal antibody to SEMA7A comprising: a heavy chain complementary determining region 3 (HC CDR3) comprising an amino acid sequence having at least 85%, at least 90%, at least 95%, at least 99%, or 100% polypeptide sequence identity to the sequence according to any polypeptide sequence represented by SEQ ID NO: 11 or SEQ ID NO: 24 and a light chain complementary determining region 3 (LC CDR3) having at least 85%, at least 90%, at least 95%, at least 99%, or 100% polypeptide sequence identity to the sequence according to any polypeptide sequence represented by SEQ ID NO: 15;
wherein the antibody further comprises a heavy chain complementary determining region 1 (HC CDR1) having at least 80%, at least 85%, at least 90%, at least 95%, at least 99%, or 100% polypeptide sequence identity to the sequence represented by SEQ ID NO: 9 and a light chain complementary determining region 1 (LC CDR1) having at least at least 80%, at least 85%, at least 90%, at least 95%, at least 99%, or 100% polypeptide sequence identity to the sequence represented by SEQ ID NO. 13; and
wherein the antibody further comprises a heavy chain complementary determining region 2 (HC CDR2) having at least at least 80%, at least 85%, at least 90%, at least 95%, at least 99%, or 100% polypeptide sequence identity to the sequence represented by SEQ ID NO: 10 and a light chain complementary determining region 2 (LC CDR2) having at least 85%, at least 90%, at least 95%, at least 99%, or 100% polypeptide sequence identity to the sequence represented by SEQ ID NO. 14.

2. The monoclonal antibody according to claim 1, wherein the antibody comprises an HC CDR3 comprising SEQ ID NO: 11 or SEQ ID NO: 24; and a LC CDR3 comprising SEQ ID NO: 15.

3. (canceled)

4. The monoclonal antibody according to claim 1, wherein the antibody further comprises a heavy chain complementary determining region 1 (HC CDR1) comprising SEQ ID NO: 9 and a light chain complementary determining region 1 (LC CDR1) comprising SEQ ID NO: 13.

5. (canceled)

6. The monoclonal antibody according to claim 1, wherein the antibody further comprises a heavy chain complementary determining region 1 (HC CDR1) comprising SEQ ID NO: 10 and a light chain complementary determining region 1 (LC CDR1) comprising SEQ ID NO: 14.

7. The monoclonal antibody according to claim 1, wherein the monoclonal antibody comprises a heavy chain variable region comprising an amino acid sequence having at least 85%, at least 90%, at least 95%, at least 99%, or 100% polypeptide sequence identity to a sequence represented by SEQ ID NO: 22 or SEQ ID NO: 5; and a light chain variable region comprising an amino acid sequence having at least 85%, at least 90%, at least 95%, at least 99%, or 100% polypeptide sequence identity to a sequence represented by SEQ ID NO: 23 or SEQ ID NO: 7.

8. The monoclonal antibody according to claim 7, wherein the heavy chain variable region comprises an amino acid sequence represented by SEQ ID NO: 22 or SEQ ID NO: 5; and the light chain variable region comprises an amino acid sequence represented by SEQ ID NO: 23 or SEQ ID NO: 7.

9. The monoclonal antibody according to claim 1, wherein the monoclonal antibody is part of a pharmaceutical composition further comprising a pharmaceutically acceptable excipient.

10. The monoclonal antibody according to claim 9, wherein the pharmaceutical composition further comprises at least one additional agent for treating a health condition.

11-15. (canceled)

16. The antibody according to claim 1, wherein the antibody is a full-length antibody, or an antigen-binding fragment thereof, a Fab, a (Fab′) 2, or a single-chain antibody.

17. The antibody according to claim 1, wherein the antibody is a full-length antibody, and wherein the full-length antibody is an IgG molecule.

18. The antibody according to claim 1, wherein the antibody comprises a full heavy chain having a polypeptide sequence comprising at least one of: at least 85%, at least 90%, at least 95%, at least 99%, or 100% polypeptide sequence identity to the polypeptide sequence represented by SEQ ID NO: 28 and a full light chain having a polypeptide sequence having at least 85%, at least 90%, at least 95%, at least 99%, or 100% polypeptide sequence identity to the polypeptide sequence represented by SEQ ID NO: 29.

19. (canceled)

20. The antibody according to claim 1, wherein the monoclonal is a human antibody or a humanized antibody.

21. The antibody according to claim 1, wherein polypeptides of the monoclonal antibody are encoded by one or more polynucleotides and the polynucleotides encoding the heavy chain variable region and the polynucleotide encoding the light chain variable region are located on a vector and the vectors comprise a single or two separate expression vectors.

22-25. (canceled)

26. The antibody according to claim 21, further comprising a host cell comprising the single or two separate vectors of the one or more-polynucleotides encoding the heavy chain variable region and the one or more polynucleotides encoding the light chain variable region.

27. The antibody according to claim 21, further comprising a hybridoma cell expressing the monoclonal antibody.

28-35. (canceled)

36. A kit comprising the antibody according to claim 1; and at least one container.

37. A method for treating a subject having a health condition comprising aberrant SEMA7A expression or overexpression, the method comprising administering to the subject a pharmaceutical composition according to claim 9 and ameliorating the health condition.

38. The method according to claim 37, wherein the health condition comprising aberrant SEMA7A expression or overexpression comprises cancer, an inflammatory condition, a cardiac condition, a fibrotic condition, or a combination thereof.

39. The method according to claim 37, wherein the health condition comprising aberrant SEMA7A expression or overexpression comprises arthritis, colitis, inflammatory bowel disease, airway inflammation, Gaucher disease, multiple sclerosis, myocardial infarction; liver fibrosis, lung fibrosis or a combination thereof.

40. The method according to claim 3937, wherein the subject is further treated with at least one other standard therapeutic agent.

41-42. (canceled)

43. The method according to claim 42, wherein the subject is further treated with at least one additional anti-cancer agent comprising one or more anti-BCL-2 or anti-PD-L1 or anti-PD-1 agent.

44. The method according to claim 38, wherein the cancer comprises breast cancer.

45. The method according to claim 37, wherein the pharmaceutical composition is administered to the subject systemically, topically, orally, intranasally subcutaneously, by bolus or by direct administration into a tumor of the subject.
